(19) United States
(12) Patent Application Publication
(10) Pub. No.: US 2008/0247987 A1
(43) Pub. Date: Oct. 9, 2008

(54) Block Copolymer Compositions and Uses Thereof

(75) Inventors: Richard T. Liggins, Coquitlam (CA); Aniko Takacs-Cox, North Vancouver (CA); David M. Gravett, West Vancouver (CA); Dechi Guan, Vancouver (CA); Troy A.E. Loss, North Vancouver (CA); Muxin Liu, Coquitlam (CA)

Correspondence Address: SEED INTELLECTUAL PROPERTY LAW GROUP PLLC, 701 FIFTH AVENUE, SUITE 5400, SEATTLE, WA, 98104-7092, US

(73) Assignee: ANGIOTECH INTERNATIONAL AG, Zug (CH)

(21) Appl. No.: 11/997,889

(22) PCT Filed: Aug. 4, 2006
(86) PCT No.: PCT/US06/30715

§ 371 Date: Jun. 11, 2008

Related U.S. Application Data
(60) Provisional application No. 60/705,737, filed on Aug. 4, 2005.

(51) Int. Cl. A61K 31/765; A61P 21/00

(52) U.S. Cl. 424/78.17

(57) ABSTRACT

The present invention describes compositions, devices, and methods for the production, use and administration of the composition having a non-thermoreversible block copolymer composition.

### BACKGROUND OF THE INVENTION

1. Field of the Invention

The present invention relates generally to pharmaceutical compositions that comprise copolymers (e.g., block copolymers such as di, tri- or multiblock copolymers) and methods for their preparation and use. The compositions may be used in a variety of medical applications, including as components of medical devices and as drug delivery systems.

2. Description of the Related Art

Formulations or compositions that include a polymer have been used for a variety of drug delivery or other medical applications. Frequently, the polymer is a copolymer (e.g., a block copolymer) formed from at least two types of repeating units (e.g., monomers). The characteristics of the formulation depend on a variety of factors, including the types of the block components of the polymer, the molecular weight of the polymer, the type and molecular weight of each segment or block in the polymer, additives, temperature, pH, and the like.

Polymeric drug delivery formulations may take a variety of physical forms. At room temperature, for example, the formulations may be in a solid, semi-solid, or liquid form. However, when introduced into or onto a tissue of a patient, the formulations may alter their properties and convert from one form into another. Drug delivery formulations may be a liquid at room temperature but may form a gel or solid or semi-solid material at physiological temperatures (e.g., upon contact with tissue). In some cases, the gel may be thermoreversible (e.g., can convert between a solid (e.g., gel) and liquid as a function of temperature). Alternatively, formulations that include a copolymer (e.g, block copolymer) and a bioactive agent may be a liquid at room temperature and remain in a liquid state when introduced into a patient.

Additional components (also referred to as “additives”) can provide polymeric drug-delivery formulations with specific physical properties, such as specific melting point, viscosity, or gel-forming properties, which properties can be advantageous for administering the formulation to a patient. Accordingly, solvents and other types of polymeric and non-polymeric additives frequently are included in drug delivery systems to alter the physical state of the formulation (e.g., to change the viscosity). For example, a solid or semi-solid polymer may be solubilized in a solvent to produce a liquid formulation.

A number of examples of compositions comprising a polymer (including some copolymers) intended for drug delivery or medical device applications have been disclosed. U.S. Pat. No. 5,384,333 discloses a composition having a bioactive agent and a biodegradable polymer, which is solid in the temperature range of 20-37° C. and requires heating to make it fluid for administration purpose. U.S. Pat. No. 5,599,552 discloses solid implants comprising a bioactive agent, thermoplastic polymer and an organic solvent. U.S. Pat. No. 6,544,544 discloses compositions comprising paclitaxel in a polymer but is limited in its disclosure of specific polymer structures that provide useful compositions. US application 2004/0001872 discloses compositions having a bioactive agent, a biodegradable polyester and a PEG or PEG derivative. US Application 2004/0185104 discloses compositions having a mixture of two triblock copolymers and paclitaxel, wherein the compositions form thermoreversible gels. U.S. Pat. No. 6,689,803 discloses methods of use of compositions comprising paclitaxel in poly(D,L-lactic-co-glycolic Acid) copolymers. Cancer Res 2000(15) 4146-51 discloses a PEG-polyester triblock copolymer combined with paclitaxel at 100 mg/g. U.S. Pat. No. 6,544,544 discloses paclitaxel in a composition comprising a polymer, including polyesters. US2002164374 discloses liquid compositions comprising both a waxy water insoluble polymer and a water soluble polymers and a hydrophobic drug. U.S. Pat. No. 6,551,610 discloses an absorbable, liquid, gel-forming composition comprising a copolymer of polyalkylene glycol end-grafted with one or more cyclic monomers. U.S. Pat. No. 5,607,686 discloses a liquid polymeric composition comprising a hydrophobic bioabsorbable polymer admixed with a hydrophilic liquid polymer. EP1125577 discloses liquid compositions containing a thermoplastic, water insoluble polymer which gels upon administration. U.S. Pat. No. 5,278,201 discloses a liquid solution of a water insoluble thermoplastic polymer and a water soluble solvent. U.S. Pat. No. 6,201,072 discloses a biodegradable and thermoreversible triblock copolymer (polyester—PEG) having MW=2000-4900, combined with paclitaxel. US 2004/0185101 discloses a liquid polymeric composition for solubilizing drug wherein the block copolymer therein has a weight averaged molecular weight of between 1500 to 3099 Daltons, WO 03/041684 discloses a copolymer system comprising benzyl alcohol as an additive, and a drug. U.S. Pat. No. 6,468,961 discloses a copolymer system comprising a benzoate as an additive, and a drug. US 2004/0001872 discloses a composition comprising a thermoreversible polyester-PEG block copolymer having a total weight average molecular weight of 1000 to 100,000 Daltons combined with a liquid PEG and a drug. U.S. Pat. No. 5,384,333 discloses a drug and copolymer composition which is solid at physiologic condition and must be melted prior to injection.

The polymers and compositions described above are limited to specific thermal properties, such as their melting temperatures, thermogelling characteristics (US2004/0001872, US2004/0185104, U.S. Pat. No. 6,201,072), and their physical forms at physiologic conditions (U.S. Pat. No. 5,599,552, U.S. Pat. No. 6,689,803, US2002/164374, U.S. Pat. No. 5,607,686, EP1125577, U.S. Pat. No. 5,278,201, WO03/1041684, U.S. Pat. No. 5,384,333). For example, some of the compositions must undergo a solid-liquid transition upon or soon after administration, while others solidify upon contact with tissues after being injected in a liquid form. These thermoreversible characteristics, though sometimes desirable, frequently prove to be a hindrance to achieving certain therapeutic goals. The limitations are notable in terms of lack of control in drug release and lack of uniformity in drug distribution in the formulation. In brief, certain specific physical properties of the polymer compositions in the art, which are largely defined by the chemical structure and molecular weight limitations of their components, make them unsuitable for a number of therapeutic applications.

There remains a need for a biodegradable drug delivery system that is a flowable liquid or can be rapidly reconstituted in an aqueous vehicle to afford a homogeneous or uniform system for easy preparation and administration of drug formulations.

### SUMMARY OF THE INVENTION

In general, the present invention provides a method of treating and preventing diseases, including cancer, bacterial infections, psoriasis, arthritis and other inflammatory conditions, fungal infections, vascular disease (e.g., restenosis and aneurysms), surgical adhesions, ocular disease and diabetes. In particular, the present invention provides treatments by administering a polymeric composition comprising a block copolymer in combination with drugs in a therapeutically effective manner.

In one aspect, the invention provides a method of treating fibrosis at a joint comprising administering to a patient in need thereof a composition comprising.

(a) a block copolymer comprising one or more blocks A and blocks B, wherein

(i) block B is more hydrophilic than block A,

(ii) the block copolymer has a molecular weight, Mn, of between about 500 g/mol and about 2000 g/mol;

(b) a non-polymeric additive; and

(c) a fibrosis-inhibiting agent,

wherein the composition is non-thermoreversible and is a liquid or semi-solid between about 20° C. to about 40° C.

In one embodiment, the fibrosis-inhibiting agent is paclitaxel.

In another embodiment, the non-polymeric additive is a low molecular weight oligomer, such as PEG300. In another embodiment, the non-polymeric additive is a surfactant.

Another embodiment provides a method of treating arthritis comprising: administering to a patient in need thereof a composition comprising.

(a) a block copolymer comprising one or more blocks A and blocks B, wherein

(i) block B is more hydrophilic than block A,

(ii) the block copolymer has a molecular weight, Mn, of between about 500 g/mol and about 2000 g/mol;

(b) a non-polymeric additive; and

(c) an anti-inflammatory agent,

wherein the composition is non-thermoreversible and is a liquid or semi-solid between about 20° C. to about 40° C.

A further embodiment provides a method of treating or preventing cartilage loss comprising: administering to a patient in need thereof a composition comprising.

(a) a block copolymer comprising one or more blocks A and blocks B, wherein

(i) block B is more hydrophilic than block A,

(ii) the block copolymer has a molecular weight, Mn, of between about 500 g/mol and about 2000 g/mol;

(b) a non-polymeric additive; and

(c) a fibrosis-inhibiting agent,

wherein the composition is non-thermoreversible and is a liquid or semi-solid between about 20° C. to about 40° C.

Yet another embodiment provides a method of treating prostate cancer comprising: administering to a patient in need thereof a composition comprising.

(a) a block copolymer comprising one or more blocks A and blocks B, wherein

(i) block B is more hydrophilic than block A,

(ii) the block copolymer has a molecular weight, Mn, of between about 500 g/mol and about 2000 g/mol;

(b) a non-polymeric additive; and

(c) an anti-microtubule agent,

wherein the composition is non-thermoreversible and is a liquid or semi-solid between about 20° C. to about 40° C.

These and other aspects of the present invention will become evident upon reference to the following detailed description and attached drawings. In addition, various references are identified below and are incorporated by reference in their entirety.

### BRIEF DESCRIPTION OF THE DRAWINGS

FIG. 1 is a bar graph showing percent (w/w) of water insoluble components in triblock copolymers following extraction into water at 37° C.

FIG. 2 is a bar graph showing percent (w/w) of water insoluble components in triblock copolymers following extraction into water at 37° C.

FIG. 3 is a bar graph showing solubility characteristics of PEG/PDLLA triblock copolymers. Max δh represents the highest δh for all solvent systems capable of dissolving the polymer at 10 mg/ml.

FIG. 4 is a bar graph showing solubility characteristics of PEG-TMC/glycolide, PEG-TMC, PPG-TMC/glycolide, and PPG-PDLLA.

FIG. 5 is a graph showing the effect of concentration of PEG400-TMC/Gly(90/10)900 in PEG 300 on paclitaxel release, expressed in terms of cumulative taxane release (% of total loading).

FIG. 6 is a graph showing the empirical relationship between the concentration of PEG 400 TMC/Gly(90/10) 900 triblock copolymer in PEG 300 and paclitaxel release over 3 days, expressed in terms of cumulative taxane release (% of total loading).

FIG. 7 is a graph showing release profiles of PEG-PDLLA triblock co-polymers with different PEG MW and polyester MW, expressed in terms of cumulative taxane release (% of total loading).

FIG. 8 is a graph showing the relationship between the molecular weight of hydrophobic blocks in triblock co-polymers and the percentage drug release in 3 days, expressed in terms of cumulative taxane release (% of total loading).

FIG. 9 is a graph showing paclitaxel release profiles for triblock copolymers (structural analogues of PEG400/TMC-Gly(90/10)900) over a period of 4 days, expressed in terms of cumulative taxane release (% of total loading).

FIG. 10 is a graph showing the relationship between the maximum Hansen Hydrogen Bonding Parameter (dh) and paclitaxel release, expressed in terms of cumulative taxane release (% of total loading).

FIG. 11 is a ternary phase diagram showing the compositions at which phase separation was observed when water was added to PEG 400 TMC/Gly(90/10) 900 triblock copolymer/PEG 300 mixtures of various compositions.

### DETAILED DESCRIPTION OF THE INVENTION

In the description that follows, a number of terms are used extensively. The following definitions are provided to facilitate understanding of the invention.

As used herein, “block copolymers” are defined as polymers having more than one polymeric block, each having a distinct structure from that of an adjacent block. The entire structure, encompassing all blocks, forms the block copolymer. A single polymeric block may itself be a copolymeric structure. For example, a diblock copolymer may comprise two distinctive blocks: block of “A” monomers and a block of alternating “A” and “B” monomers, represented by “AAAAAAA-BABABABABAB”. A diblock copolymer may also contain monomers “A”, “B” and “C”, for example, in the form of “BBBBCCCCBBBBCCCC-AAAAAAAA”. In this case, the block copolymer contains a block of “A” monomer and a block that itself contains blocks of “B” and “C”. This copolymer may also be characterized as a multiblock copolymer, having five blocks, one “A” block, two “B” blocks and two “C” blocks.

A “triblock copolymer” has three distinct blocks, preferably of alternating hydrophobic (A) and hydrophilic (B) blocks. An exemplary triblock copolymer has an ABA-type structure, such as [polyester]-[polyalkylene oxide]-[polyester], where polyester is hydrophobic and polyalkylene oxide is hydrophilic. Either of the A or B blocks may, themselves, be a copolymer.

Block copolymers may have a variety of molecular weights. In certain embodiments, the block copolymer may comprise a polymer having a bi or multimodal molecular weight distribution, where higher and lower molecular weight fractions are present. In certain embodiments, the copolymer may comprise a polymer with fractions having varying proportions of block length or monomer content, for example an A-B diblock copolymer comprising 60% by weight of polymer chains with 90% mol/mol A and 10% mol/mol B and 40% by weight of polymer chains with 50% mol/mol A and 50% mol/mol B.

As used herein, a “blend” is a mixture of two or more components characterized by the lack of, or substantial lack of, covalent bonding between the components.

As used herein, a “polymeric blend” is a mixture of two biodegradable, biocompatible polymers, in which one polymer is water insoluble and the other polymer is water soluble. An example of a polymeric blend is a mixture of a water insoluble triblock copolymer and a water soluble polyalkylene oxide.

“Thermoreversible” or “thermoreversible gel” or “thermoreversible polymer,” as used herein, refers to a substance (e.g., a polymer or a solution of a polymer) that exists as a relatively low viscosity liquid at low temperature (e.g., room temperature) and forms a more viscous liquid or gel at a higher temperature (e.g., 37° C.). The increase in viscosity (also referred to as gelation) of the polymer occurs through non-covalent interactions between the polymer chains (e.g., van der Waals or hydrogen bonding) as a function of temperature. These interactions are reversible, such that lowering the temperature decreases the viscosity of the substance which induces the gel to revert to a liquid form.

Polymers considered to be “thermoreversible” may be naturally occurring polymers, synthetic polymers, and combinations thereof. Representative examples of thermoreversible substances that form thermoreversible gels include aqueous solutions of PLURONIC® polymers (available from BASF Corporation, Mount Olive, N.J.), collagen, gelatin, hyalouronic acid, and polysaccharides.

“Non-thermoreversible” or “non-thermoreversible polymer,” or “non-reversible” as used herein, refers to a substance (e.g., a polymer or a solution of a polymer) that exists as a relatively low viscosity liquid at low temperature (e.g., room temperature) and remains a liquid at physiological temperatures (e.g., 37° C. to 42° C.). The viscosity of the liquid may remain the same or become reduced upon heating of the substance. Alternatively, a “non-thermoreversible” material may be a relatively low viscosity liquid at low temperature (e.g., room temperature) and forms a gel at higher temperatures (e.g., 37° C. to 42° C.)). The resultant gel, however, does not revert to its initial low viscosity state by lowering the temperature.

A “drug” or “bioactive agent” or “therapeutic agent” is a therapeutically active substance which is delivered to a living subject to produce a desired effect, such as to treat a condition of the subject. A drug is also provided to a subject prophylactically to prevent or inhibit the development of a condition or to decrease the severity of a condition that the subject may develop.

As used herein, a “hydrophobic drug,” is a water insoluble drug. A “water insoluble drug” has a solubility of less than 0.1 mg/mL in distilled water at 25° C. Within the context of the present invention, a “slightly soluble drug” (solubility: 1-10 mg/mL) and a “very slightly soluble drug” (solubility: 0.1-1 mg/mL) may also be referred to. These terms are well-known to those of skill in the art. See, e.g., Martin (ed.), Physical Pharmacy, Fourth Edition, page 213 (Lea and Febiger 1993). Exemplary hydrophobic drugs include certain steroids, such as budesonide, testosterone, progesterone, estrogen, flunisolide, triamcinolone, beclomethasone, betamethasone; dexamethasone, fluticasone, methylprednisolone, prednisone, hydrocortisone, and the like; certain peptides, such as cyclosporin cyclic peptide, retinoids, such as all-cis retinoic acid, 13-trans retinoic acid, and other vitamin A and beta carotene derivatives; vitamins D, E, and K and water insoluble precursors and derivatives thereof; prostaglandins and leukotrienes and their activators and inhibitors including prostacyclin (epoprostanol), and prostaglandins; tetrahydrocannabinol; lung surfactant lipids; lipid soluble antioxidants; hydrophobic antibiotics and chemotherapeutic drugs such as amphotericin B and adriamycin and the like.

As used herein, “a polymeric drug delivery system,” is a polymer or polymer blend having a hydrophobic drug dissolved, suspended or otherwise distributed within one or more polymers.

The term “slow release” refers to the release of a drug from a polymeric drug delivery system over a period of time that is more than one day.

The term “additive” as used herein refers to a substance that is included into a copolymer formulation for a specific purpose. The additive may be essential to the formation or existence of the formulation or may serve an auxiliary or secondary function. To that end, an additive may be incorporated in order to achieve optimization of properties including: color, odor, texture or other cosmetic factors, viscosity, solubility characteristics, sterility, bacteriostatic properties, chemical or physical stability of the composition, mechanical strength, or flexibility, characteristics relating to the release of a bioactive agent, such as release rate, or burst phase, biocompatibility, cytotoxicity, efficacy of the composition for its intended purpose, chemical or physical compatibility of certain other components of the composition, surfactant properties, melting point or glass transition temperature, crystallinity, liquid crystallinity, swelling, dissolution, gellation or hydrogenation properties, viscosity, strength or elasticity. Exemplary additives include antioxidants, thickeners, plasticizers, stiffeners, preservatives or bacteriostatic agents, bactericidal agents, coloring agents, dyes, and the like. In particular, an additive may be incorporated into a formulation to modulate mechanical or other physical dispositions of the copolymer composition, such as viscosity, degree of cross-linking, degree of bioadhesion, release kinetics of a bioactive agent, or to facilitate an in situ reaction. For example, an additive may function as an adjuvant or an excipient and may be a polymeric or a non-polymeric substance.

“Adjuvant” refers to a substance that, when included in a therapeutic composition (e.g., a composition that includes one or more bioactive agents), will improve or enhance the therapeutic efficacy of one or more of the bioactive agents contained in the composition. The adjuvant may enhance the overall therapeutic effectiveness of the composition or may, for example, counteract a negative side effect (e.g, stability or toxicity) associated with the therapeutic composition.

“Excipient” refers to an inert or substantially inert, non-toxic substance present in a therapeutic composition which can confer some benefit to the composition, such as improved physical and/or chemical stability or improved handling characteristics (e.g., flowability and consistency). The excipient may, for example, function as a bulking agent, i.e., a material that reduces the concentration of the bioactive agent in the therapeutic composition.

A “non-polymeric additive” refers to an additive that does not include a polymer. For purpose of this invention, a polymer is defined as a macromolecule, natural or synthetic, formed by the chemical union of at least 10 repeating monomers and has a molecular weight of at least 500 g/mol. A non-polymeric additive may be an inorganic material, an organic material or a semi-synthetic material. In certain aspects, a “non-polymeric additive” is a molecule without a generally repetitive structure. There is no particular limitation to the molecule weight of this type of non-polymeric additive. Examples include preservatives, colorant, stabilizer, excipients for providing texture (e.g., abrasives or microabrasives), and excipients for providing a cooling or heating sensation (e.g., camphor).

In certain other aspects, a non-polymeric additive may be an oligomer. An “oligomer” or an “oligomer additive” as used herein refers to a molecular chain having more than one repeating units but its molecular weight (less than 500) is too small to be considered as a polymer. In one aspect, an oligomer has fewer than 10 repeating monomeric units. A typical example of an oligomer additive is polyethylene glycol (PEG) or polypropylene glycol (PPG), both having fewer than 10 repeating ether units with molecular weight less than 500. An oligomer additive can be a liquid at 20° C. Additionally, an oligomer additive can be a non-ionic surfactant or emulsifier such as a fatty alcohol or wax. These surfactants contain more than 10 repeating units of —CH₂— and nevertheless are less than 500 in molecular weight. Examples include glyceryl stearate, PEG 75 stearate, cetyl alcohol (C16) and stearyl alcohol (C18).

“Stabilizer” refers to an excipient that improves the physical or chemical stability (e.g., the storage stability) of the therapeutic composition. The stabilizer assists in maintaining the therapeutic efficacy of the active agent(s) present in the therapeutic compositions. An exemplary stabilizer is an “antioxidant”, where this term refers to synthetic or natural substances that prevent or delay the oxidative deterioration of a bioactive agent. Exemplary antioxidants include lecithin, gamma oryzanol; ubiquinone (ubidecarenone) and coenzyme Q; vitamins, such as vitamins A, C (ascorbic acid) and E and beta-carotene; natural components such as carnosol, carnosic acid and rosmanol found in rosemary and hawthorn extract, proanthocyanidins, such as those found in grapeseed or pine bark extract, and green tea extract.

“Fibrosis,” “scarring,” or “fibrotic response” refers to the formation of fibrous tissue in response to injury or medical intervention.

Therapeutic agents which inhibit fibrosis or scarring are referred to herein as “anti-fibrotic agents,” “fibrosis-inhibiting agents,” “anti-scarring agents,” and the like, where these agents inhibit fibrosis through one or more mechanisms including: inhibiting angiogenesis, inhibiting migration or proliferation of connective tissue cells (such as fibroblasts, smooth muscle cells, vascular smooth muscle cells), reducing ECM production, and/or inhibiting tissue remodeling.

“Inhibit fibrosis,” “reduce fibrosis,” and the like are used synonymously to refer to the action of agents or compositions which result in a statistically significant decrease in the formation of fibrous tissue that can be expected to occur in the absence of the agent or composition.

Therapeutic agents which promote (also referred to interchangeably herein as induce, stimulate, cause, increase, accelerate, and the like) fibrosis or scarring are referred to interchangeably herein as “fibrosis-inducing agents,” “scarring agents,” “fibrosing agents,” “adhesion-inducing agents,” and the like. These agents promote fibrosis through one or more mechanisms including, for example, inducing or promoting angiogenesis, stimulating migration or proliferation of connective tissue cells (such as fibroblasts, smooth muscle cells, vascular smooth muscle cells), inducing extracellular matrix (ECM) production, and promoting tissue remodeling. In addition, numerous therapeutic agents described herein can have the additional benefit of promoting tissue regeneration (the replacement of injured cells by cells of the same type).

“Host,” “person,” “subject,” “patient” and the like are used synonymously to refer to the living being into which the compositions provided herein are administered.

“Inhibitor” refers to an agent that prevents a biological process from occurring or slows the rate or degree of occurrence of a biological process. The process may be a general one such as scarring or refer to a specific biological action such as, for example, a molecular process resulting in release of a cytokine.

“Anti-microtubule agents” should be understood to include any protein, peptide, chemical, or another molecule that impairs the function of microtubules, for example, through the prevention or stabilization of polymerization. Compounds that stabilize polymerization of microtubules are referred to herein as “microtubule stabilizing agents.” A wide variety of methods may be utilized to determine the anti-microtubule activity of a particular compound, including for example, assays described by Smith et al., (Cancer Lett 79(2):213-219, 1994) and Mooberry et al., (Cancer Lett. 96(2):261-266, 1995).

“Medical device,” “implant,” “medical device or implant,” “implant/device” and the like are used synonymously to refer to any object that is designed to be placed partially or wholly within a patient's body for one or more therapeutic or prophylactic purposes such as for restoring physiological function, alleviating symptoms associated with disease, delivering therapeutic agents, and/or repairing, replacing or augmenting damaged or diseased organs and tissues.

“Bioresorbable” as used herein refers to the property of a composition or material being able to be cleared from a body after administration to a human or animal. Bioresorption may occur by one or more of a variety of means, such as, for example, dissolution, oxidative degradation, hydrolytic degradation, enzymatic degradation, metabolism, clearance of a component, its breakdown product, or its metabolite through routes such as, for example, the kidney, intestinal tract, lung or skin.

“Biodegradable” refers to materials for which the degradation process is at least partially mediated by, and/or performed in, a biological system. “Degradation” refers to a chain scission process by which a polymer chain is cleaved into oligomers and monomers. Chain scission may occur through various mechanisms, including, for example, by chemical reaction (e.g., hydrolysis) or by a thermal or photolytic process. Polymer degradation may be characterized, for example, using gel permeation chromatography (GPC), which monitors the polymer molecular mass changes during erosion and drug release. Biodegradable also refers to materials may be degraded by an erosion process mediated by, and/or performed in, a biological system. “Erosion” refers to a process in which material is lost from the bulk. In the case of a polymeric system, the material may be a monomer, an oligomer, a part of a polymer backbone, or a part of the polymer bulk. Erosion includes (i) surface erosion, in which erosion affects only the surface and not the inner parts of a matrix; and (ii) bulk erosion, in which the entire system is rapidly hydrated and polymer chains are cleaved throughout the matrix. Depending on the type of polymer, erosion generally occurs by one of three basic mechanisms (see, e.g., Heller, J., CRC Critical Review in Therapeutic Drug Carrier Systems (1984), 1(1), 39-90); Siepmann, J. et al., Adv. Drug Del. Rev. (2001), 48, 229-247): (1) water-soluble polymers that have been insolubilized by covalent cross-links and that solubilize as the cross-links or the backbone undergo a hydrolytic cleavage; (2) polymers that are initially water insoluble are solubilized by hydrolysis, ionization, or protonation of a pendant group; and (3) hydrophobic polymers are converted to small water-soluble molecules by backbone cleavage. Techniques for characterizing erosion include thermal analysis (e.g., DSC), X-ray diffraction, scanning electron microscopy (SEM), electron paramagnetic resonance spectroscopy (EPR), NMR imaging, and recording mass loss during an erosion experiment. For microspheres, photon correlation spectroscopy (PCS) and other particles size measurement techniques may be applied to monitor the size evolution of erodible devices versus time.

“Solid” refers a substance having a structure of a rigid and defined geometry, which is readily deformable when pressure is applied. “Semi-solid” refers to a substance having a structure of defined geometry to the extent that it is not freely flowable. A semi solid, however, is not rigid and can be deformed upon pressure. Examples of semi-solid substances include gel, paste, paste, ointment and cream. As used herein, the “semi solid” typically has a viscosity of at least 100,000 cP (centipoises) at 20° C. “Liquid” refers to a substance that is freely flowable. As used herein, the liquid typically has a viscosity of no more than 100,000 cP at 20° C.

A “gel” as used herein refers to a semi-solid and has some property of a liquid (the shape is resilient and deformable) and some of the properties of a solid (i.e., the shape is discrete enough to maintain three dimensions on a two dimensional surface.) It can be further characterized by relatively high yield values as described in Martin's Physical Pharmacy (Fourth Edition, Alfred Martin, Lea & Febiger, Philadelphia, 1993, pp 574-575). Gels may contain non-crosslinked materials and possess certain properties, such as elevated viscosity and elasticity, which may be reduced with increased dilution with an aqueous medium, such as water or buffer. Gels with sufficiently low viscosity are injectable.

Certain polymers may be crosslinked to form systems that are herein defined as “hydrogels.” A hydrogel will maintain an elevated level of viscosity and elasticity when diluted with an aqueous solution, such as water or buffer. Crosslinking may be accomplished by different means including covalent, hydrogen, ionic, hydrophobic bonding, chelation, complexation, and the like.

“Carrier” as used herein refers to any of a number of matrices, solid, semi-solid or liquid which can be made to contain a block copolymer composition that is an embodiment of this invention. The carrier may be a continuous phase, such as a suspension or a gel, or may be a plurality of matrices, such as a microparticle having a coating. The carrier may be biologically derived and may include living tissue.

“Scaffold” as used herein refers to any structure, solid or semi-solid upon or in which a block copolymer composition with or without a carrier can be positioned. A scaffold may be formed in situ.

As used herein, the following terms are given the indicated abbreviations: poly(ε-caprolactone) (PCL); polyesters (PE); polyethylene glycol (PEG); polyglycolide (PGA); polylactide (PLA); poly(lactide-co-glycolide) (PLGA); and poly(DL-lactide-co-ε-caprolactone) (PLC), trimethylene carbonate (TMC).

### Non-Thermoreversible Block Copolymer Compositions

Generally speaking, in one aspect, the present invention provides a therapeutic composition comprising:

(a) block copolymer comprising one or more blocks A and blocks B, wherein
(i) block B is more hydrophilic than block A,
(ii) the block copolymer has a molecular weight, Mn, of between about 500 g/mol and about 2000 g/mol,

(b) optionally a non-polymeric additive; and

(c) a bioactive agent,

wherein the composition is non-thermoreversible and is a liquid or semi-solid between about 20° C. to about 42° C.

The composition of the present invention may be, for example, a homogenous solution or a suspension, emulsion, or dispersion of one or more phases in another. Bioactive agents may be incorporated into the compositions of the invention by various methods, such as being contained (e.g., dissolved or dispersed) within the block copolymer matrix. The composition may further include a carrier that can be formed into solid or semi-solid forms, such as a gel, a hydrogel, particles (e.g. microspheres, nanospheres) a suspension, a paste, a cream, an ointment, a tablet, a spray, a powder, an orthopedic implant, fibers, a fabric, a gauze or a pledget.

In one aspect, the block copolymer itself is a liquid. In one embodiment, the block copolymer is a liquid above about 4° C. In one embodiment, the block copolymer is a liquid above about 20° C. In one embodiment, the block copolymer is a liquid at a physiological temperature, which is about 35° C. to 42° C.

In another aspect, the block copolymer itself is a semi-solid or solid, however, upon being blended with a non-polymeric additive, such as a low molecular weight PEG, the composition or blend becomes a liquid within the relevant temperature range of about 20° C. to 42° C. In a further aspect, the block copolymer and a low molecular weight PEG form a semi-solid, such as a gel, as defined herein.

The block copolymer composition, optionally containing a non-polymeric additive, is non-thermoreversible. In other words, the composition exhibits no melting transition within relevant temperature range (about 20° C. to 42° C.) such that the composition maintain its liquid or semi-solid from at room temperature and physiological conditions.

The compositions of the present invention contain copolymers of substantially low molecular weight than those typically used in polymeric drug delivery systems. As a result, a less viscous and more rapidly clearing formulation may be achieved. In one embodiment, the copolymer has a viscosity of below about 30,000 cP at 35° C. In another embodiment, the copolymer has a viscosity of below about 1,000 cP at 35° C. In one embodiment, the composition has a viscosity of below about 150 cP at 25° C.

In one aspect, the composition of the present invention is insoluble in aqueous condition. In other aspect, the composition is partially soluble in aqueous condition, which characteristic provides that certain segments of the copolymer and/or the non-polymeric additive readily dissolves and releases portions of the therapeutic agent in a “burst phase”.

In a further aspect of the invention, the block copolymer composition comprises two phases, whereby a block copolymer having hydrophobic and hydrophilic blocks, an optional non-polymeric additive and a bioactive agent form a liquid first phase. The composition further comprises a second phase, which is immiscible with the first phase. In one embodiment, the second phase is a liquid. In certain embodiment, the liquid second phase comprises water. In other embodiment, the liquid second phase does not comprise water. In other embodiments, the second phase comprises a carrier, as defined here. In further embodiments, the second phase is semi-solid or solid. Typical forms of solid or semi-solid include a gel, a hydrogel, a suspension, a paste, a cream, an ointment, a tablet, a spray, a powder, an orthopedic implant, a fabric, a gauze or a pledget.

In certain embodiments, the block copolymer comprises at least 50% w/w of the composition. In various other embodiments, the block copolymer comprises less than 50%, 25%, 10%, 5% and 1% w/w of the composition.

The characteristics of each components are described in details below.

### A. Block Copolymer:

The block copolymers of the present invention can be broadly defined by any combination of the following attributes: (1) the number of blocks, (2) the order or arrangement of blocks, (3) the total molecular weight, (4) the ratio and type of monomers, (5) the ratio of block lengths or weights, (6) the point of attachment of blocks (e.g. linear, branched or star copolymer blocks), (7) the amount of block copolymer in the composition, and (8) the ratio of bioactive agent to copolymer.

Copolymers may be described by a variety of nomenclatures. Herein, general polymer naming conventions are followed and abbreviations are defined. Specific diblock and triblock structures are described as follows. For diblock copolymers, the more hydrophilic block is generally named first followed by its molecular weight, e.g., MePEG 500 denotes methoxypolyethylene glycol having a molecular weight of 500 g/mol. This is followed by the more hydrophobic block with its molecular weight. For example, MePEG 500-PDLLA 900 denotes a diblock copolymer having a more hydrophilic block of MEPEG, MW=500 g/mol, and a more hydrophobic block of poly(DL-lactide), MW=900 g/mol, giving a polymer with total molecular weight of 1400 g/mol. For triblock copolymers of the type B-A-B the center block “A” is named first with its molecular weight followed by the external blocks “B” with their combined molecular weight. For example, “PEG 200-PCL 900 triblock copolymer” denotes a triblock having a center block of polyethylene glycol MW=200 g/mol, linked at each end with poly(ε-caprolactone), both external chains having a total molecular weight of 900 g/mol, or an average of 450 g/mol each. When an individual block in a copolymer is itself a copolymer, its structure is defined in brackets prior to its molecular weight. For example, PEG 400-TMC/Gly (90/10) 900 is a triblock copolymer (which may be inferred by the fact that the hydrophilic block is a di-functional PEG), having a center block of PEG with MW=400 g/mol and external blocks having a molar ratio of trimethylene carbonate (TMC) and glycolide (Gly) of 90:10 and a total molecular weight of 900 g/mol, or an average of 450 g/mol per block.

In certain embodiments, the copolymer may comprise a polymer having a bi- or multimodal molecular weight distribution, for example, a higher and lower molecular weight fraction. In certain embodiments, the copolymer may comprise a polymer with fractions having varying proportions of block length or monomer content, for example, an A-B diblock copolymer comprising 60% by weight of polymer chains with 90% mol/mol A and 10% mol/mol B and 40% by weight of polymer chains with 50% mol/mol A and 50% mol/mol B.

In certain embodiments, a block copolymer, such as a triblock copolymer, may have structural limitations to provide for a specific functional requirement. For example, the total molecular weight of the polymer may be sufficiently low so that the polymer is a liquid at 25° C., or have a specified maximum viscosity (e.g., 150 cP) at 25° C. Such a molecular weight may be, for example, about 2000 g/mol or less, or about 1400 g/mol or less, or about 1000 g/mol or less, or about 900 g/mol or less. In other embodiments, the molecular weight of a specific block within the polymer may be specified to impart a specific characteristic, such as glass transition temperature, crystallinity, mechanical properties or drug releasing properties. For example, the molecular weight of an A block in a A-B-A polymer may be specified as being at most about 200, 400, 600, 800, 1000, 2000 g/mol, and/or the molecular weight of each B block may be specified as being at most about 100, 200, 300, 400, 500, 600, 700, 800, 900, 1000, 1,500, 2,000 g/mol.

In certain embodiments, the block copolymer comprises one or more blocks A and block B where block B is more hydrophilic than block A. In certain embodiments, the block copolymer has a molecular weight of between about 500 g/mol and about 2000 g/mol. In certain embodiments, the block copolymer is a triblock copolymer, optionally comprising a carbonate monomer. In certain embodiments, the triblock copolymer has an average molecular weight of between about 600 and about 1500 g/mol.

Hydrophilic blocks may comprise a polyalkylene oxide, for example, polyethylene glycol or polypropylene glycol, poly(1-4-butanediol), or a copolymer thereof (e.g., random, alternating or block copolymers). These hydrophilic blocks may be reactive at more than one site (e.g., at two sites or more than two sites) or may be capped at one or more sites to generate less reactive sites for the preparation of diblock copolymers. Hydrophilic blocks may have molecular weights that range from between about 100 to 2000 g/mol. Exemplary molecular weight ranges for hydrophilic blocks can be from about 200-500 g/mol (e.g., about 200, 300, 340, 350, 400, 425 g/mol), or about 500-2000 g/mol (e.g., about 600, 725, 750, 1000, 2000 g/mol). Monomers suitable for the preparation of copolymers having hydrophilic blocks include materials known to those skilled in the art, such as propylene glycol, butane diol, ethylene glycol, and the like.

Hydrophobic blocks may comprise a biodegradable polymer or copolymer of one or more of the monomers D-lactide, L-lactide, D,L-lactide, glycolide, ε-caprolactone, trimethylene carbonate, d and g valerolactones and butyrolactones, d-decanolactone, 1,4-dioxane-2-one, 1,5-dioxepan-2-one, caprolactams or trimethylene carbonates. The hydrophobic blocks therefore include polyesters such as poly(L-lactide) poly(D,L lactide), poly(D,L-lactide-co-glycolide), poly(L-lactide-co-glycolide), poly(lactic acid-co-glycolic acid), poly(ε-caprolactone-co-lactide), poly(trimethylene carbonate-co-glycolide), poly(glycolide-co-ε-caprolactone) and poly(lactide-co-1,4-dioxane-2-one). These hydrophobic blocks may be coupled to one or more reactive sites of a hydrophilic block to form a diblock or triblock copolymers. Hydrophobic blocks may have molecular weights that range from between about 100 to 2000 g/mol. Exemplary molecular weight ranges for hydrophobic blocks can be from about 200-500 g/mol (e.g., about 200, 300, 340, 350, 400, 425 g/mol), or about 500-2000 g/mol (e.g., about 600, 725, 750, 1000, 2000 g/mol).

In certain embodiments, the block polymer is an ABA triblock copolymer wherein the B block comprises a polyalkylene oxide (e.g., polyethylene glycol) and the A blocks comprise a polymer having about a 90:10 mole ratio of trimethylene carbonate (TMC) and glycolide (Gly) residues. In certain embodiments, the B block has a molecular weight of between about 200 g/mol to about 600 g/mol (e.g., about 400 g/mol), and/or the A blocks have a total molecular weight of from about 700 g/mol to 1100 g/mol (e.g., about 900 g/mol).

In certain aspects, the block copolymer is non-thermoreversible. In one embodiment, the block copolymer is a liquid at room temperature (about 20° C.), and remains a liquid at physiological conditions (about 35° C. to 42° C.) In another embodiment, the block copolymer is a semi-solid at room temperature (about 20° C.), and becomes a liquid at physiological conditions (about 35° C. to 42° C.)

In some embodiments, the relative balance of hydrophobic (A) block(s) to hydrophilic (B) block(s) may have a specified limit, to impart properties such as drug releasing characteristics or water solubility. Solubility characteristics depend in part on the identity of the solvents or cosolvent systems in which the polymer dissolves, the type of the blocks, the molecular weight of the overall copolymer and/or of each type of block, the relative weight fraction (w/w %) of the hydrophilic blocks, and the presence of low molecular weight fractions.

Solubility characteristics may be described in terms of the percent by mass of the polymer that is soluble in a given solvent. Such percentage can be ascertained by measuring the molecular weight of the polymer before and after a purification process, such as exposing the polymer to a polar solvent (e.g., water) to remove the lower molecular weight or more hydrophilic fractions (or more hydrophobic fraction in the case when the solvent is non-polar.) In certain embodiments, a polymer has a water soluble fraction that is less than 1, 5, 10, 25, 30, 40, 50, 60, 75, 80, or 90% w/w. In certain embodiments, polymers with a low % w/w water soluble fraction, i.e., the polymer being poorly soluble or insoluble in water, may be used to form depot matrices for the administration of a therapeutic agent. Depot matrices that include a therapeutic agent as described herein can provide for prolonged delivery of the therapeutic agent in a patient. In other embodiments, polymers with a higher water soluble fraction may be desirable. For example, polymers with a higher water soluble fraction greater than about 50% or greater than about 80%, or that is completely water soluble (100%), which are combined with a therapeutic agent, may be used to readily disperse the therapeutic agent upon administration to a patient.

Solubility may also be characterized in terms of the identity of solvents or co-solvent systems in which the polymer dissolves, e.g., at a concentration of 10, 20 or 50 mg/ml. Solubility may be further described in terms of the solubility parameters in which the polymer dissolves at its specified concentration level. Solubility parameters may include the interaction parameter C, Hildebrand solubility parameter d, or partial (Hansen) solubility parameters: δp, δh and δd, describing the solvent's polarity, hydrogen bonding potential and dispersion force interaction potential, respectively. In certain embodiments, the highest value for a solubility parameter that describes a solvent or co-solvent system in which the polymer will dissolve may provide a limitation for the polymer. For example, a triblock or diblock polymer that will not completely dissolve at 10 or 20 mg/ml in solvents that have a characteristic δh value greater than 23 may be suitable for some applications. Yet, in other applications, a higher value may be preferred. Higher values will have a greater hydrogen bonding ability and would therefore have a greater affinity for solvent molecules such as water. A higher value of maximum observed δh for a solvent could be preferred for cases where a more hydrophilic polymer is required.

In one embodiment, the copolymer dissolves in a solvent having a δh Hansen solubility parameter value of no less than 22. In another embodiment, the copolymer dissolves in a solvent, wherein the copolymer has a δh Hansen solubility parameter value of no less than 32. In other embodiments, the copolymer dissolves in a solvent, wherein the copolymer has a δh Hansen solubility parameter value of no less than 42. In further embodiments, the copolymer and the bioactive agent have respective δh Hansen solubility parameter values, and the difference between said respective δh Hansen solubility parameter values does not exceed 5.

### B. Bioactive Agents or Drugs

Therapeutic composition of the present invention may include a wide variety of bioactive agents (used interchangeably with “drugs” and “therapeutic agents”). In certain embodiments of the invention, the drugs may be selected from a variety of therapeutically active compounds for which controlled or sustained release may provide a benefit to the patient.

Representative examples of classes of therapeutic agents (which are efficacious in one of a number of indications) include, for example, vitamins, anti-infectives, anti-inflammatories, anticancer agents, immunosuppressants, antihistamines, antipsychotics, antiangiogenic compounds, analgesics, diuretics, lipid or cholesterol lowering agents, anticoagulants, anticonvulsants, anti-thrombotic agents, profibrotic agents, anti-fibrotic agents, fibrosing agents, vasoconstrictors, vasodilators, antiarrhythmics, narcotics, narcotic antagonists, antibiotics, retinols, sedatives, stimulants, thyroid stimulants, thyroid hormone suppressants, labor inducing agents, sunscreens, blood glucose level modifying compounds, or neuromuscular blockers or relaxants. In certain embodiments, the therapeutic agent has at least one of anti-inflammatory, antibiotic, anti-infective, anti-microtubule, anti-fibrotic, fibrosis-inducing, antioxidant, anti-restontic, anticancer activity, and neurological or anaesthetic activities.

The present compositions may include any number of hydrophobic and/or hydrophilic drugs. For example, compositions are described that include a drug with a water solubility at 25° C. of less than 10% (weight of drug/volume of water), less than 2% (w/v), less than 1% (w/v), or less than 0.75% (w/v), less than 0.5% (w/v), or less than 0.1% (w/v) as measured by techniques such as quantitative chromatography, and spectroscopic methods such as UV or IR absorption. Typically, a bioactive agent is considered as a “hydrophobic drug” if it is insoluble or sparingly or poorly soluble in water. As used herein, such drugs will have a solubility below 10 mg/ml, usually below 1 mg/ml, sometimes below 0.01 mg/ml, and sometimes below 0.001 mg/ml.

Examples of hydrophobic drugs that could be used in this polymeric drug delivery system, or with the ABA triblock copolymers, include the following.

Amphotericin can be used for the treatment or prevention of infection of an open wound by topical administration or for the treatment or prevention of an infection in an exposed wound after surgery by local application. Amphotericin is an antifungal and is insoluble in water at pH 6 to 7. See, e.g., The Merck Index.

Anthralin can be used for the treatment of “wet” psoriasis by topical application. Anthralin is an agent for psoriasis therapy and is practically insoluble in water. See, e.g., The Merck Index.

Beclomethasone can be used for the reduction of local inflammation by peri-ophthalmic and inside the eyelid or intranasal (e.g., for the treatment of rhinitis) application. Beclomethasone is a corticosteroid and is very slightly soluble in water. See, for example, Gennaro, (ed.), Remington's Pharmaceutical Sciences, 17th Edition, (Mack Publishing Company 1985).

Betamethasone is used for the reduction of local inflammation by oral (e.g., canker sore), intravaginal, and intrarectal application. Betamethasone is a corticosteroid and has a solubility of 190 μg/mL water. See, for example, Gennaro, (ed.), Remington's Pharmaceutical Sciences, 17th Edition, (Mack Publishing Company 1985).

Camptothecin is used for the treatment of diseases involving cellular proliferation such as cancer, arthritis, psoriasis, restenosis, and surgical adhesions. Camptothecin has a water solubility of 1-2 μg/mL.

Curcumin is a potent antioxidant and is under investigation as an anti-arthritic drug. Curcumin is practically insoluble in water.

Dexamethasone is used for the reduction of local inflammation by oral application (e.g., post wisdom tooth removal). Dexamethasone is a corticosteroid and has a solubility of 10 μg/mL in water. See, e.g., The Merck Index.

Indomethacin is used for the treatment of symptoms of gout by intraarticular or intramuscular injection, or for the reduction of local inflammation by peri-ophthalmic and inside the eyelid, oral, intranasal, intravaginal and intrarectal application. Indomethacin is a non-steroidal anti-inflammatory (NSAID) and is practically insoluble in water. See, e.g., The Merck Index.

Genistein is a tyrosine kinase inhibitor and is under investigation for the treatment of diseases involving cellular proliferation. Genistein is practically insoluble in water.

Lidocaine provides local anesthesia by intramuscular injection, or administration by application to mucus membranes, including periophthalmic and inside the eyelid, oral, intranasal, intravaginal and intrarectal. Lidocaine is a local anesthetic and is practically insoluble in water. See, for example, Gennaro, (ed.), Remington's Pharmaceutical Sciences, 17th Edition, (Mack Publishing Company 1985).

Proteins that are practically insoluble in water, such as insulin, can be used in the presently described polymeric drug delivery system.

Paclitaxel is used for the treatment of angiogenic related diseases such as arthritis, cancer, restenosis, psoriasis, or surgical adhesions. Paclitaxel has a water solubility of 1-2 μg/mL.

Tetracycline is used for the treatment of eye infections by peri-ophthalmic and inside the eyelid application. Tetracycline is an antibacterial and has a solubility of 400 μg/mL water. See, e.g., Gennaro, (ed.), Remington's Pharmaceutical Sciences, 17th Edition, (Mack Publishing Company 1985).

Tretinoin is a retinoic acid that is being investigated as an anticancer agent. Tretinoin is practically insoluble in water.

The block copolymer composition of the present invention may be loaded with drugs having any molecular weight. In certain embodiments, block copolymer compositions are described which include a drug having a molecular weight of greater than 445 g/mol (e.g., paclitaxel, rapamycin, geldanamycin and its analogues, etoposide, vancomycin, vincristine and its analogues). In certain embodiments, the compound has at least 23 carbon atoms (e.g., paclitaxel, angiotensisn, polymyxin, oxytocin, docetaxel, codeine, irinotecan, vitamins E and D, cephalosporines, buprinorphine, loperamide, raloxifene, beclomethasone, hydrocortisone, interferons, somatotropins, and certain bioactive peptides). In certain embodiments, block copolymer compositions are described which include 50% (w/w) or greater of a drug having a molecular weight of less than 180 g/mol (e.g., pyrimidine derivatives such as 5-fluorouracil, phenol derivatives such as silver fluoride (MW=127), phenylpropanolamine (MW=151), nicotinic acid (MW=123), flucytrosine (MW=129), tryptamine (MW=160), salicylic acid (sodium salt) (MW=160) and fenadiazole (MW=162)).

1. Fibrosing Agents

In certain embodiments, the drug may be an agent that promotes fibrosis or scarring. Therapeutic agents that promote fibrosis or scarring can do so through one or more mechanisms including: inducing or promoting angiogenesis, stimulating migration or proliferation of connective tissue cells (such as fibroblasts, smooth muscle cells, vascular smooth muscle cells), inducing ECM production, and/or promoting tissue remodeling. In addition, numerous therapeutic agents described in this invention will have the additional benefit of also promoting tissue regeneration (the replacement of injured cells by cells of the same type). Fibrosis-inducing agents are described, e.g., in the U.S. patent application entitled “Medical Implants and Fibrosis-Inducing Agents,” filed Nov. 20, 2004 (U.S. Ser. No. 10/986,230) and in the U.S. patent application entitled “Compositions and Methods for Treating Diverticular Disease,” filed May 12, 2005 (U.S. Ser. No. 11/129,763), both applications are incorporated by reference in their entireties. Exemplary fibrosing agents include, but are not limited to, silk (such as silkworm silk, spider silk, recombinant silk, raw silk, hydrolyzed silk, acid-treated silk, and acylated silk), fibroin, seracin, talc, chitosan, polylysine, fibronectin, bleomycin or an analogue or derivative thereof, a fibrosing agent that connective tissue growth factor (CTGF), metallic beryllium or an oxide thereof, copper, saracin, silica, crystalline silicates, quartz dust, talcum powder, ethanol, a component of extracellular matrix, collagen, fibrin, fibrinogen, poly(ethylene terephthalate), poly(ethylene-co-vinylacetate), N-carboxybutylchitosan, an RGD protein, a polymer of vinyl chloride, cyanoacrylate, crosslinked poly(ethylene glycol)-methylated collagen, an inflammatory cytokine, TGFβ, PDGF, VEGF, TNFα, NGF, GM-CSF, IGF-a, IL-1, IL-8, IL-6, a growth hormone, a bone morphogenic protein, a cell proliferative agent, dexamethasone, isotretinoin, 17-β-estradiol, estradiol, diethylstilbestrol, cyclosporine a, all-trans retinoic acid or an analogue or derivative thereof, wool (including animal wool, wood wool, and mineral wool), cotton, bFGF, polyurethane, polytetrafluoroethylene, poly(alkylcyanoacrylate), activin, angiopoietin, insulin-like growth factor (IGF), hepatocyte growth factor (HGF), a colony-stimulating factor (CSF), erythropoietin, an interferon, endothelin-1, angiotensin II, bromocriptine, methylsergide, fibrosin, fibrin, an adhesive glycoprotein, proteoglycan, hyaluronan, secreted protein acidic and rich in cysteine (SPaRC), a thrombospondin, tenacin, a cell adhesion molecule, an inhibitor of matrix metalloproteinase, a tissue inhibitor of matrix metalloproteinase, methotrexate, carbon tetrachloride, and thioacetamide.

2. Fibrosis-Inhibiting Agents

In certain embodiments, the drug may be an agent that inhibits fibrosis or scarring. The term “fibrosis-inhibiting”, “anti-fibrotic”, “anti-scarring” agents are used interchangeably. Therapeutic agents which inhibit fibrosis or scarring can do so through one or more mechanisms including: inhibiting angiogenesis, inhibiting migration or proliferation of connective tissue cells (such as fibroblasts, smooth muscle cells, vascular smooth muscle cells), reducing ECM production, and/or inhibiting tissue remodeling. In addition, numerous therapeutic agents described in this invention will have the additional benefit of also reducing tissue regeneration (the replacement of injured cells by cells of the same type) when appropriate. Fibrosis-inhibiting agents are described, e.g., in U.S. patent application, “Medical Implants and Anti-Scarring Agents,” filed Nov. 10, 2004 (U.S. Ser. No. 10/986,231); and “Anti-Scarring Agents, Therapeutic Compositions, and Use Thereof,” filed May 10, 2005 (U.S. Ser. No. 60/679, 293). Exemplary anti-fibrotic agents include, but are not limited to, cell cycle inhibitors (e.g., doxorubicin, mitoxantrone, TAXOTERE, vinblastine, tubercidin, paclitaxel, and analogues and derivatives thereof), podophyllotoxins (e.g., etoposide), immunomodulators (e.g., sirolimus and everolimus), heat shock protein 90 antagonists (e.g., geldanamycin) and analogues and derivatives thereof, HMGCoA reductase inhibitors (e.g., simvastatin) and analogues and derivatives thereof, inosine monophosphate dehydrogenase inhibitors (e.g., mycophenolic acid, 1-alpha-25 dihydroxy vitamin D₃) and analogues and derivatives thereof, NF kappa B inhibitors (e.g., Bay 11-7082) and analogues and derivatives thereof, antimycotic agents (e.g., sulconizole) and analogues and derivatives thereof, p38 MAP kinase inhibitors (e.g., SB202190) and analogues and derivatives thereof, and anti-angiogenic agents (e.g., halofuginone bromide) and analogues and derivatives. Additional exemplary anti-fibrotic agents include, but are not limited to, ZD-6474 (an angiogenesis inhibitor), AP-23573 (an mTOR inhibitor), synthadotin (a tubulin antagonist), S-0885 (a collagenase inhibitor), aplidine (an elongation factor-1 alpha inhibitor), ixabepilone (an epithilone), IDN-5390 (an angiogenesis inhibitor and an FGF inhibitor), SB-2723005 (an angiogenesis inhibitor), ABT-518 (an angiogenesis inhibitor), combretastatin (an angiogenesis inhibitor), anecortave acetate (an angiogenesis inhibitor), SB-715992 (a kinesin antagonist), temsirolimus (an mTOR inhibitor), adalimumab (a TNFα antagonist), erucylphosphocholine (an ATK inhibitor), alphastatin (an angiogenesis inhibitor), BXT-51072 (an NF Kappa B inhibitor), etanercept (a TNFα antagonist and TACE inhibitor), humicade (a TNFα inhibitor), and gefitinib (a tyrosine kinase inhibitor), as well as analogues and derivatives of the aforementioned.

3. Anti-Inflammatory Agents and Analgesics

In certain embodiments, the drug to be incorporated into block copolymer compositions of the present invention may have anti-inflammatory activity or analgesic activity. In these embodiments, the drug may be one or more non-steroidal anti-inflammatory agents (including aspirin, ibuprofen, indomethacin, naproxen, prioxicam, diclofenac, tolmetin, fenoclofenac, meclofenamate, mefenamic acid, etodolac, sulindac, carprofen, fenbufen, fenoprofen, flurbiprofen, ketoprofen, oxaprozin, tiaprofenic acid, phenylbutazone diflunisal, salsalte, and salts and analogues thereof); opiates (including codeine, meperidine, methadone, morphine, pentazocine, fentanyl, hydromorphone, oxycodone, oxymorphone, and salts and analogues thereof).

In other embodiments, the drug may be selected from one or a combination of steroidal anti-inflammatory agents. Examples of steroidal anti-inflammatory agents include without limitation: hydrocortisone and esters thereof, methylprednisolone, amoxapine and the like. In one embodiment, the drug incorporated may be an anti-inflammatory agent such as naproxen or indomethacin. In yet other embodiments, the anti-inflammatory agent is ketoprofen or an analogue or derivative thereof.

4. Antibiotic and Anti-Infective Agents

In certain embodiments, the bioactive agent may be an antibiotic or anti-infective agent, which may act by a number of mechanisms. They may be anthelmintics (including mebendazole, niclosamide, piperazine, praziquante, thibendazole and pyrantel pamoate); aminoglycosides (including tobramycin, gentamicin, amikacin and kanamycin); antifungals (including amphotericin B, clotrimazole, fluconazole, ketoconazole, itraconazole, miconazole, nystatin, and griseofulvin); cephalosporins (including cefazolin, cefotaxime, cefoxitin, defuroxime, cefaclor, cefonicid, cefotetan, cefoperazone, ceftriaxone, cephalexin, moxalactam, and ceftazidime, and salts thereof); β-lactams (including aztreonam, and imipenem); chloramphenicol and salts thereof; erythromycins and salts thereof (including roxithromycin, erythromycin, and its esters such as ethylsuccinate, guceptate and stearate); penicillins (including penicillin G, amoxicillin, amdinocillin, ampicillin, carbenicillin, ticarcillin, cloxacillin, nafcillin, penicillin V, and their salts and esters); tetracyclines (including tetracycline, and doxycycline, and salts thereof); clindamycin; polymixin B; vancomycin; ethambutol; isoniazid; rifampin; rifampicin; antivirals (including acyclovir, zidovudine, vidarabine); anti-HIV drugs; quinolones (including ciprofloxacin); sulfonamides; nitrofurantoin; metronidazole; clofazimine; triclosan and chlorhexidine. Antibiotic agents also include active analogues and derivatives of the aforementioned antibiotic agents. In certain embodiments, the antibiotic of the invention has additional therapeutic activities as anticancer and/or anti-restenotic activities.

In certain embodiments, the drug incorporated may be an antibiotic such as a sulfonamide.

5. Anti-Microtubule Agents

A wide variety of anti-microtubule agents can be utilized in the present invention to form high drug loading microparticles. Representative examples of anti-microtubule agents include taxanes, colchicine, LY290181, glycine ethyl ester, aluminum fluoride, and CI-980 (Allen et al., Am. J. Physiol 261(4 Pt. 1): L315-L321, 1991; Ding et al., J. Exp. Med. 171(3): 715-727, 1990; Gonzalez et al., Exp. Cell. Res. 192(1): 10-15, 1991; Stargell et al., Mol. Cell. Biol. 12(4): 1443-1450, 1992; Garcia et al., Antican. Drugs 6(4): 533-544, 1995), vinca alkaloids (e.g., vinblastine and vincristine), discodermolide (ter Haar et al., Biochemistry 35: 243-250, 1996), as well as analogues and derivatives of any of these (see also PCT/CA97/00910 (WO 98/24427), which as noted above is hereby incorporated by reference in its entirety, for a list of additional anti-microtubule agents).

Within one embodiment of the invention, the anti-microtubule agent is paclitaxel, a compound that disrupts mitosis (M-phase) by binding to tubulin to form abnormal mitotic spindles, or an analogue or derivative thereof.

The utility of the anti-microtubule agent paclitaxel, as a component of the compositions that comprise part of this invention, is demonstrated by data from a series of in vitro and in vivo experiments. Paclitaxel inhibits neutrophil activation (Jackson et al., Immunol. 90:502-10, 1997), decreases T-cell response to stimuli, and inhibits T-cell function (Cao et al., J. Neuroimmunol 108:103-11, 2000), prevents the proliferation of and induces apoptosis in synoviocytes (Hui et al, Arth. Rheum. 40:1073-84, 1997), inhibits AP-1 transcription activity via reduced AP-1 binding to DNA (Hui et al., Arth. Rheum. 41:869-76, 1998), inhibits collagen induced arthritis in an animal model (Brahn et al, Arth. Rheum. 37:839-45, 1994; Oliver et al., Cellular Immunol. 157:291-9, 1994) but is non-toxic to non-proliferating cells, such as normal chondrocytes and non-proliferating synoviocytes (Hui et al., Arth. Rheum. 40:1073-84, 1997).

Paclitaxel, formulations, prodrugs, epimers, isomers, analogues and derivatives thereof may be readily prepared utilizing techniques known to those skilled in the art (see, e.g., Schiff et al., Nature 277:665-667, 1979; Long and Fairchild, Cancer Research 54:4355-4361, 1994; Ringel and Horwitz, J. Nat'l Cancer Inst. 83(4):288-291, 1991; Pazdur et al., Cancer Treat. Rev. 19(4):351-386, 1993; WO 94/07882; WO 94/07881; WO 94/07880; WO 94/07876; WO 93/23555; WO 93/10076; WO94/00156; WO 93/24476; EP 590267; WO 94/20089; U.S. Pat. Nos. 5,294,637; 5,283,253; 5,279,949; 5,274,137; 5,202,448; 5,200,534; 5,229,529; 5,254,580; 5,412,092; 5,395,850; 5,380,751; 5,350,866; 4,857,653; 5,272,171; 5,411,984; 5,248,796; 5,248,796; 5,422,364; 5,300,638; 5,294,637; 5,362,831; 5,440,056; 4,814,470; 5,278,324; 5,352,805; 5,411,984; 5,059,699; 4,942,184; Tetrahedron Letters 35(52):9709-9712, 1994; J. Med. Chem. 35:4230-4237, 1992; J. Med. Chem. 34:992-998, 1991; J. Natural Prod. 57(10):1404-1410, 1994; J. Natural Prod. 57(11):1580-1583, 1994; J. Am. Chem. Soc. 110:6558-6560, 1988), or obtained from a variety of commercial sources, including for example, Sigma Chemical Co., St. Louis, Mo. (T7402- from Taxus brevifolia).

Representative examples of paclitaxel derivatives or analogues include 7-deoxy-docetaxol, 7,8-cyclopropataxanes, N-substituted 2-azetidones, 6,7-epoxy paclitaxels, 6,7-modified paclitaxels, 10-desacetoxytaxol, 10-deacetyltaxol, phosphonoxy and carbonate derivatives of taxol, taxol 2′,7-di(sodium 1,2-benzenedicarboxylate, 10-desacetoxy-11,12-dihydrotaxol-10,12(18)-diene derivatives, prodrugs including 2′- and/or 7-O-ester, amide, thioester derivatives, (2′- and/or 7-O-carbonate derivatives), fluoro taxols, 9-deoxotaxol, 7-deoxy-9-deoxotaxol, 10-desacetoxy-7-deoxy-9-deoxotaxol, sulfonated 2′-acryloyltaxol and sulfonated 2′-O-acyl acid taxol derivatives, succinyltaxol, 2′-γ-aminobutyryltaxol formate, 2′-acetyl taxol, 7-acetyl taxol, 7-glycine carbamate taxol, 2′-OH-7-PEG(5000) carbamate taxol, 2′-benzoyl and 2′,7-dibenzoyl taxol derivatives, other prodrugs (2′-acetyltaxol; 2′,7-diacetyltaxol; 2′-succinyltaxol; 2′-(beta-alanyl)-taxol); 2′-γ-aminobutyryltaxol formate; ethylene glycol derivatives of 2′-succinyltaxol; prodrugs or derivatives having amino acids attached at either or both of the 2′ and 7 positions (R₉ and R₃, respectively); 2′-glutaryltaxol; 2′-(N,N-dimethylglycyl)taxol; 2′-(2-(N,N-dimethylamino)propionyl)taxol; 2′-orthocarboxybenzoyl taxol; 2′-aliphatic carboxylic acid derivatives of taxol, prodrugs {2′-(N,N-diethylaminopropionyl)taxol, 2′(N,N-dimethylglycyl)taxol, 7(N,N-dimethylglycyl)taxol, 2′,7-di-(N,N-dimethylglycyl)taxol, 7(N,N-diethylaminopropionyl)taxol, 2′,7-di(N,N-diethylaminopropionyl)taxol, 2′-(L-glycyl)taxol, 7-(L-glycyl)taxol, 2′,7-di(L-glycyl)taxol, 2′-(L-alanyl)taxol, 7-(L-alanyl)taxol, 2′,7-di(L-alanyl)taxol, 2′-(L-leucyl)taxol, 7-(L-leucyl)taxol, 2′,7-di(L-leucyl)taxol, 2′-(L-isoleucyl)taxol, 7-(L-isoleucyl)taxol, 2′,7-di(L-isoleucyl)taxol, 2′-(L-valyl)taxol, 7-(L-valyl)taxol, 2′7-di(L-valyl)taxol, 2′-(L-phenylalanyl)taxol, 7-(L-phenylalanyl)taxol, 2′,7-di(L-phenylalanyl)taxol, 2′-(L-prolyl)taxol, 7-(L-prolyl)taxol, 2′,7-di(L-prolyl)taxol, 2′-(L-lysyl)taxol, 7-(L-lysyl)taxol, 2′,7-di(L-lysyl)taxol, 2′-(L-glutamyl)taxol, 7-(L-glutamyl)taxol, 2′,7-di(L-glutamyl)taxol, 2′-(L-arginyl)taxol, 7-(L-arginyl)taxol, 2′,7-di(L-arginyl)taxol}, TAXOL (Bristol-Myers Squibb Company, New York, N.Y.) analogues with modified phenylisoserine side chains, taxotere, (N-debenzoyl-N-tert-(butoxycaronyl)-10-deacetyltaxol, cephalomannine, Taxol C, Taxol D, Taxol E, Taxol F, brevifoliol, yunantaxusin and taxusin, debenzoyl-2-acyl paclitaxel derivatives, benzoate paclitaxel derivatives, sulfonated 2′-acryloyltaxol; sulfonated 2′-O-acyl acid paclitaxel derivatives, C18-substituted paclitaxel derivatives, chlorinated paclitaxel analogues, C4 methoxy ether paclitaxel derivatives, sulfenamide taxane derivatives, brominated paclitaxel analogues, Girard taxane derivatives, nitrophenyl paclitaxel, 10-deacetylated substituted paclitaxel derivatives, C7 taxane derivatives, C10 taxane derivatives, 2-debenzoyl and 2-acyl paclitaxel derivatives, taxane analogues bearing new C2 and C4 functional groups, n-acyl paclitaxel analogues, 10-deacetyl taxol B, and 10-deacetyl taxol, benzoate derivatives of taxol, 2-aroyl-4-acyl paclitaxel analogues, ortho-ester paclitaxel analogues, and deoxy paclitaxel and deoxy paclitaxel analogues.

In one aspect, the anti-microtubule agent is a taxane having the formula (C1):

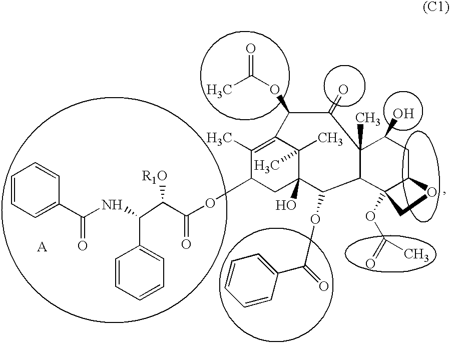

where the gray-highlighted portions may be substituted and the non-highlighted portion is the taxane core. A side-chain (labeled “A” in the diagram) is desirably present in order for the compound to have good activity as an anti-microtubule agent. Examples of compounds having this structure include paclitaxel (Merck Index entry 7117), docetaxol (TAXOTERE, Merck Index entry 3458, Aventis Pharma S.A., France), and 3′-desphenyl-3′-(4-ntirophenyl)-N-debenzoyl-N-(t-butoxycarbonyl)-10-deacetyltaxol.

In certain embodiments, suitable taxanes such as paclitaxel and its analogues and derivatives are disclosed in U.S. Pat. No. 5,440,056 as having the structure (C2):

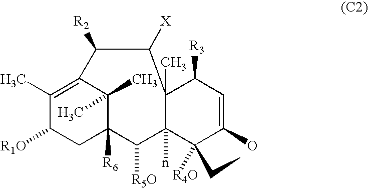

wherein X may be oxygen (paclitaxel), hydrogen (9-deoxotaxol or 9-deoxy derivatives, which may be further substituted to yield taxanes such as 7-deoxy-9-deoxotaxol, 10-desacetoxy-7-deoxy-9-deoxotaxol,), thioacyl, or dihydroxyl precursors; R₁ is selected from paclitaxel or taxotere side chains or an alkanoyl of the formula (C3)

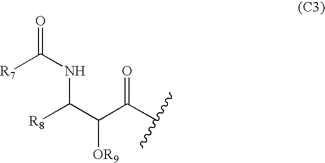

wherein R₇ is selected from hydrogen, alkyl, phenyl, alkoxy, amino, phenoxy (substituted or unsubstituted); R₈ is selected from hydrogen, alkyl, hydroxyalkyl, alkoxyalkyl, aminoalkyl, phenyl (substituted or unsubstituted), alpha or beta-naphthyl; and R₉ is selected from hydrogen, alkanoyl, substituted alkanoyl, and aminoalkanoyl; where substitutions refer to hydroxyl, sulfhydryl, allalkoxyl, carboxyl, halogen, thioalkoxyl, N,N-dimethylamino, alkylamino, dialkylamino, nitro, and —OSO₃H, and/or may refer to groups containing such substitutions; R₂ is selected from hydrogen or oxygen-containing groups, such as hydrogen, hydroxyl, alkoyl, alkanoyloxy, aminoalkanoyloxy, and peptidyalkanoyloxy to yield taxanes that include in some cases with further substitution: 10-deacetyltaxol, 10-desacetoxy-11,12-dihydrotaxol-10,12(18)-diene derivatives, 10-deacetyl taxol A, 10-deacetyl taxol B; R₃ is selected from hydrogen or oxygen-containing groups, such as hydrogen, hydroxyl, alkoyl, alkanoyloxy, aminoalkanoyloxy, and peptidyalkanoyloxy, and may further be a silyl containing group or a sulphur containing group; R₄ is selected from acyl, alkyl, alkanoyl, aminoalkanoyl, peptidylalkanoyl and aroyl; R₅ is selected from acyl, alkyl, alkanoyl, aminoalkanoyl, peptidylalkanoyl and aroyl; R₆ is selected from hydrogen or oxygen-containing groups, such as hydrogen, hydroxyl alkoyl, alkanoyloxy, aminoalkanoyloxy, and peptidyalkanoyloxy.

In certain embodiments, the paclitaxel analogues and derivatives useful as anti-microtubule agents in the present invention are disclosed in PCT International Patent Application No. WO 93/10076. As disclosed in this publication, the analogue or derivative should have a side chain attached to the taxane nucleus at C₁₃, as shown in the structure below (formula C4), in order to confer antitumor activity to the taxane.

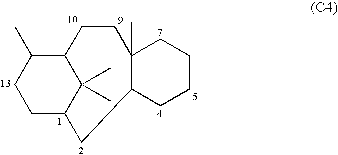

WO 93/10076 discloses that the taxane nucleus may be substituted at any position with the exception of the existing methyl groups. The substitutions may include, for example, hydrogen, alkanoyloxy, alkenoyloxy, aryloyloxy. In addition, oxo groups may be attached to carbons labeled 2, 4, 9, 10. As well, an oxetane ring may be attached at carbons 4 and 5. As well, an oxirane ring may be attached to the carbon labeled 4.

In one aspect, the taxane-based anti-microtubule agent useful in the present invention is disclosed in U.S. Pat. No. 5,440,056, which discloses 9-deoxo taxanes. These are compounds lacking an oxo group at the carbon labeled 9 in the taxane structure shown above (formula C4). The taxane ring may be substituted at the carbons labeled 1, 7 and 10 (independently) with H, OH, O—R, or O—CO—R where R is an alkyl or an aminoalkyl. As well, it may be substituted at carbons labeled 2 and 4 (independently) with aryol, alkanoyl, aminoalkanoyl or alkyl groups. The side chain of formula (C3) may be substituted at R₇ and R₈ (independently) with phenyl rings, substituted phenyl rings, linear alkanes/alkenes, and groups containing H, O or N. R₉ may be substituted with H, or a substituted or unsubstituted alkanoyl group.

In one embodiment, the anti-microtubule agent is a taxane (e.g., paclitaxel or an analogue or derivative thereof).

6. Cardiovascular and Anti-Restenotic Agents

In certain embodiments, therapeutic drugs may be agents that inhibit some or all of the processes involved in the development of intimal hyperplasia, such as cell proliferation, cell migration and matrix deposition. Agents in this category include cell cycle inhibitors and/or anti-angiogenic agents (e.g., anthracyclines and taxanes), immunosuppressive compounds (e.g., sirolimus and its analogues and derivatives), and non-steroidal anti-inflammatory agents (e.g., dexamethasone and its analogues and derivatives). Furthermore, antithrombotic agents and antiplatelet agents may also be loaded into the block copolymer composition.

In certain embodiments, the therapeutic agent is sirolimus, or a derivative or an analogue thereof. Sirolimus (also referred to as “rapamycin”) is a macrolide antibiotic. Sirolimus analogues useful in the present invention include tracolimus and derivatives thereof (e.g., EP0184162B1 and U.S. Pat. No. 6,258,823), and everolimus and derivatives thereof (e.g., U.S. Pat. No. 5,665,772). Further representative examples of sirolimus analogues and derivatives include ABT-578 and others can be found in PCT Publication Nos. WO 97/10502, WO 96/41807, WO 96/35423, WO 96/03430, WO 96/00282, WO 95/16691, WO 95/15328, WO 95/07468, WO 95/04738, WO 95/04060, WO 94/25022, WO 94/21644, WO 94/18207, WO 94/10843, WO 94/09010, WO 94/04540, WO 94/02485, WO 94/02137, WO 94/02136, WO 93/25533, WO 93/18043, WO 93/13663, WO 93/11130, WO 93/10122, WO 93/04680, WO 92/14737, and WO 92/05179. Representative U.S. patents include U.S. Pat. Nos. 6,342,507, 5,985,890, 5,604,234, 5,597,715, 5,583,139, 5,563,172, 5,561,228, 5,561,137, 5,541,193, 5,541,189, 5,534,632, 5,527,907, 5,484,799, 5,457,194, 5,457,182, 5,362,735, 5,324,644, 5,318,895, 5,310,903, 5,310,901, 5,258,389, 5,252,732, 5,247,076, 5,225,403, 5,221,625, 5,210,030, 5,208,241, 5,200,411, 5,198,421, 5,147,877, 5,140,018, 5,116,756, 5,109,112, 5,093,338, and 5,091,389.

7. Anticancer Agents

Anticancer agents suitable to be incorporated into block copolymer compositions of the present invention may act by a number of mechanisms. These agents may be antimetabolites, anti-microtubule agents, chelating agents, antibiotics or antiangiogenic agents. Exemplary anticancer agents useful in the present invention include, but are not limited to, alkylating agents such as bis(chloroethyl)amines (including cyclophosphamide, mechlorethamine, chlorambucil, or melphalan), nitrosoureas (including carmustine, estramustine, lomustine or semustine), aziridines (including thiotepa or triethylenemelamine), alkylsulfonates including busulfan, other agents with possible alkylating agent activity (including procarbazine, cisplatin, carboplatin, dacarbazine, or hexamethylmelamine); antimetabolites such as methotrexate, mercaptopurine, thioguanine, 5-fluorouracil, cytarabine, azacitidine; plant alkaloids such as vinca alkaloids (including vincristine, vinorelbine, or vinblastine), bleomycin, dactinomycin, anthracyclines (including daunorubicin or doxorubicin, idarubicin, epirubicin, pirarubicin, zorubicin carubicin, anthramycin, mitoxantrone, menogaril, nogalamycin, aclacinomycin A, olivomycin A, chromomycin A₃, and plicamycin), etoposide, teniposide, mithramycin, mitomycin; hormonal agents such as androgens (including testosterone, or fluoxymestrone), antiandrogens including flutamide, estrogens (including diethylstilbesterol, estradiol, ethylestradiol, or estrogen), antiestrogens including tamoxifen, progestins (including hydroxyprogesterone, progesterone, medroxyprogesterone, or megestrol acetate), adrenocorticosteroids (including hydrocortisone, or prednisone), gonadotropin-releasing hormones and agonists thereof including leuprolide; cytadrenl other anticancer agents (including amscarine, asparaginase, hydroxyurea, mitotane, quniacrine); and anti-microtubule agents including paclitaxel and docetaxol. Also included are analogues and derivatives of the aforementioned compounds. Additional anticancer agents may be defined as compounds which exhibit therapeutic activity against cancer, as defined using standard tests known in the art, including in vitro cell studies, in vivo and ex vivo animal studies and clinical human studies. Suitable tests are described in texts such as “Anticancer Drug Development Guide” (B. A. Teicher ed., Humana Press, 1997 Totowa, N.J.). Other anticancer agents include antiangiogenic agents such as active taxanes as described above, including paclitaxel and docetaxol; angiostatic steroids including squaline; cartilage derived proteins and factors; thrombospondin; matrix metalloproteinases (including collagenases, gelatinases A and B, stromelysins 1, 2 and 3, martilysin, metalloelastase, MT1-MMP (a progelatenase), MT2-MMP, MT3-MMP, MT4-MMP, Bay 12-9566 (Bayer), AG-3340 (Agouron), CGS27023! (Novartis), Chiroscience compounds D5140, D1927, D2163); and phytocemicals (including genistein, daidzein, leuteolin, apigenin, 3 hydroxyflavone, 2′,3′-dihydroxyflavone, 3′,4′-dihydroxyflavone, or fisetin). Anti-angiogentic agents also include active analogues and derivatives of the aforementioned antiangiogenic agents. Certain anticancer agents are also classified as antifibrotic agents. These include mitomycin C₁₋₅-fluorouracil, interferons, D-penicillamine and β-aminopropionitrile.

8. Neurologically Active Agents

In certain embodiments of the invention, the drug incorporated into block copolymer compositions in a high loading is neurologically active. Such drugs may have the following therapeutic activities: anticonvulsants, antipsychotics, anaesthetics and antidepressants, anti-Parkinson's disease compounds, and anti-Alzheimer's disease compounds. Exemplary anticonvulsants include barbiturates (such as secobarbital, phenobarbital, amobarbital and primidone); benzodiazepines such as clonazepam; hydantoins such as phenyloin; succinimides such as ethosuximide, and valproic acid. Exemplary antidepressants include tricyclic antidepressants such as amitriptylline, desipramine, doxepin, imipramine, nortriptylline, protriptyline, and trimipramine; heterocyclics such as maprotiline, nefazodone, venlafaxine, amoxapine, trazodone, alprazolam, and fluoxetine and chlropropiophenones such as bupropion; and serotonin reuptake inhibitors such as fluoxetine, fluvoxamine, and paroxetine. Antipsychotic agents include haloperidol, loxapine, molindone, perphenazine, thioridazine, trifluoperazine, thiotixene, chlorpromazine, and fluphenazine. Exemplary anaesthetics include methohexital sodium, thiopental sodium, etomidate, keatmine, propofol, bupivicaine, chloroprocaine, etidocaine, lidocaine, mepivicaine, prilocaine, procaine, tetracaine, benzocaine, cocaine, dibucainem dyclonnine, and pramoxine. Exemplary anti-Parkinson's disease compounds include selegiline (L-deprenyl). Salts (for example hydrochlorides and sodium salts), esters, prodrugs, analogues and derivatives of the aforementioned compounds are additional exemplary neurologically active agents.

Other drugs useful in the present invention include immunomodulatory agents such as cyclosporine A and mycophenolic acid, including analogues, ester prodrugs and derivatives thereof; drugs useful in treating certain lung disorders, such as theophylline or pentoxyffyline. The drug incorporated in the microsphere may also be an aesthetic such as lidocaine, xylocalne, etidocaine, carobicaine, xylocalne, marcaine, nesacaine, etiod, or bupivicaine. For example, block copolymer compositions are described containing about 40% (e.g., lidocaine) to greater than about 80% (e.g., bupivicaine).

9. Antioxidant Agents

Antioxidant agents suitable to be incorporated into block copolymer compositions of the present invention may act by a number of mechanisms. They may be vitamins (e.g., vitamins C and E) or quinolone compounds (e.g., BHA and BHT), amino acids (e.g., N-acetylcysteine), a metal or metal containing molecule or salt having an antioxidant metal such as selenium, cadmium, zinc or vanadium, particularly metals with a +2 valence, other compounds such as repaglinide, carnosine, antioxidant extracts or fractions thereof from green or black teas, alpha-lipoic acid, or antioxidant enzymes. Particularly suitable antioxidants include hydrophobic molecules having a melting point above 40° C., including analogs and derivatives of the aforementioned antioxidants.

### C. Additives

In certain embodiments of the invention, the compositions may contain one or more additive components, which may be essential to the formation or existence of the formulation or may serve an auxiliary or secondary function, such as to homogenize the formulation. The additive of the present invention is non-polymeric in nature that can be incorporated into the present compositions to alter the mechanical or physical properties of the composition, such as viscosity, degree of cross-linking (in the case of hydrogels), degree of bioadhesion, release kinetics of a bioactive agent, or to facilitate some in situ reaction.

Viscosity of a block copolymer composition may be modulated by the addition of an oligomer, as defined herein. An oligomer additive of the present invention has a molecular weight less than 500. More typically, an oligomer additive has a molecular weight less than 400.

In one aspect, an oligomer has less than 10 repeating units of a monomer. Such an oligomer is typically liquid within temperature range of 20° C. to 42° C., and the addition of which tends to reduce the viscosity of the block copolymer composition. Examples of this type of oligomers include PEG 200, PEG 300, PEG 400 and PPG 425.

In other aspects, an oligomer additive may also have more than 10 repeating units of monomers, e.g., —CH₂—, but whose overall molecular weight remains less than 500. Viscosity may be modulated by the inclusion of such an additive, which functions as a stiffener. Examples include a wax, a viscous oil, a fatty alcohol and uni- or multivalent ionic species.

In situ reactions may be facilitated by the addition of pH adjusters, also discussed below. pH adjusters may take the form of acids, bases or buffers. Suitable acids include acetic acid, hydrochloric acid and benzoic acid. Suitable bases include sodium hydroxide, triethylamine. Various buffers based on phosphate, lactate and carbonate salts may also be employed to obtain pH values within the compositions, or parts of the composition in the range of 2 to 11.

i. Liquid Oligomer

As discussed above, an oligomer such as PEG, PEG derivative, PPG, and PPG derivative can be added to the block copolymer to modulate the overall properties of the composition. Principally, this type of oligomeric additive functions as a liquid diluent, in which the block copolymer can be uniformly distributed. A “diluent” as used herein refers to a chemical compound, usually a liquid, which dissolves or disperses a compound of interest thereby reducing the concentration of the compound to less than that of the compound alone. The block copolymer in the presence of such an oligomer diluent typically afford a composition of reduced viscosity as compared to the viscosity of the block copolymer alone. In one embodiment, the composition has a viscosity of less than 3000 cP at 25° C. In another embodiment, the composition has a viscosity of less than 1000 cP at 25° C. In another embodiment, the composition has a viscosity of less than 150 cP at 25° C.

The oligomer additive such as PEG, PPG and their derivatives are typically of low molecular weight of less than 500. Typically, the additive has a molecular weight of between about 100 g/mol and about 500 g/mol. More typically, the additive has a molecular weight of between about 200 g/mol and about 400 g/mol.

The oligomer additives of the present invention are liquid at room temperature (20° C.) and remains so at physiological conditions (37° C. to 42° C.). The additive thus reduces the viscosity of the block copolymer composition and forms injectable and rapidly clearing formulations. Typically, the block copolymer is present in composition having a liquid oligomer additive at a w/w concentration of 2.5%, 5%, 10%, 20%, 33% and 50%.

ii. Surfactant

In one aspect, the compositions of the present invention may include a surfactant. In certain aspects, the surfactant may be non-ionic surfactant. Representative examples of non-ionic surfactants include, for example, sorbitan monolaurate NF (e.g., ARLACEL 20); sorbitan monopalmitate NF (ARLACEL 40), sorbitan monostearate NF (ARLACEL 60); sorbitan monooleate NF (ARLACEL 80); sorbitan sesquioleate (ARLACEL 83); sorbitan sesquioleate (ARLACEL C); glycerol monostearate and polyoxyethylene stearate (acid-stable, self-emulsilying) (ARLACEL 165); glycerol monostearate and polyoxyethylene stearate (acid-stable, self-emulsilying) (ARLACEL 165v); glycerol monooleate diluted with propylene glycol (ARLACEL 186); ethoxylated glycerol sorbitan unsaturated fatty acid ester (ARLACEL 581); ethoxylated glycerol sorbitan saturated fatty acid ester (ARLACEL 582); Sorbitan monosostearate (ARLACEL 987); ethoxylated hydrogenated castor oil (ARLACEL 989); Polymeric surfactant (ARLACEL P100); polymeric surfactant (ARLACEL P135); all available from ICI Americas, Inc. (Wilmington, Del.); polyoxyethylene 20 isohexadecyl ether (ARLASOLVE 200 Liquid) available from ICI Americas, Inc. (Wilmington, Del.); polyoxyethylene 20 isohexadecyl ether (ARLATONE 200); ARLATONE DUO; ARLATONE MAP; polyoxyethylene 25 hydrogenated castor oil (ARLATONE G); ethoxylated fatty alcohol (ARLATONE 985); sorbitol and sugar esters (ARLATONE 2121); polyoxyethylene 40 sorbital septaoleate (ARLATONE 1); all available from ICI Americas, Inc. (Wilmington, Del.); ethoxylated castor oil (ATLAS G-1284); PEG 6 sorbitan beeswax (ATLAS G-1702); PEG 20 sorbitan beeswax (ATLAS G-1726); polyoxyethylene 25 propylene glycol steat (ATLAS G-2162); polyoxyethylene 80 sorbitan mono (ATLAS G-4280); all available from Atlas Pharma Corporation (Palm Beach, Fla.); polyoxyethylene 4 lauryl ether (BRIJ 30); BRIJ 35; polyoxyethylene 23 lauryl ether (BRIJ 35 Liquid); polyoxyethylene 23 lauryl ether (BRIJ 35 SD); polyoxyethylene 2 cetyl ether (BRIJ 52); polyoxyethylene 10 cetyl ether (BRIJ 56); polyoxyethylene 20 cetyl ether (BRIJ 58); polyoxyethylene 20 cetearyl ether (BRIJ 68); polyoxyethylene 2 stearyl ether (BRIJ 72); polyoxyethylene 10 stearyl ether (BRIJ 76); polyoxyethylene 20 stearyl ether (BRIJ 78); polyoxyethylene 2 oleyl ether (low (BRIJ 93); polyoxyethylene 10 oleyl ether (low (BRIJ 97); polyoxyethylene 20 oleyl ether (BRIJ 98); polyoxyethylene 100 stearyl ether (BRIJ 700); polyoxyethylene 21 stearyl ether (BRIJ 721, 721S), all available from ICI Americas, Inc. (Wilmington, Del.); n-soya-n-ethyl morpholinium ethosulphate 35% aqueous solution (FORESTALL); available from ICI Americas, Inc. (Wilmington, Del.); polyoxyl 8 stearate (polyoxyethylene 8 stearate) (MYRJ 45); polyoxyethylene 40 stearate, NF (polyoxyethylene 40 stearate (MYRJ 52); polyoxyethylene 40 stearate, NF (polyoxyethylene 40 stearate) (MYRJ 52FL); polyoxyethylene 40 stearate, NF (MYRJ 52S); polyoxyethylene 50 stearate, NF (MYRJ 53); polyoxyethylene 100 stearate (MYRJ 59); polyoxyethylene 100 stearate (MYRJ 59FL), all available from ICI Americas, Inc. (Wilmington, Del.); sorbitan monolaurate, NF (SPAN 20); sorbitan monoplamilate, NF (SPAN 40); sorbitan monostearate, NF (SPAN 60, 60K); sorbitan tristearate (SPAN 65); sorbitan monooleate, NF (SPAN 80); sorbitan trioleate (SPAN 85), all available from Wako Chemicals USA, Inc. (Richmond, Va.); and polysorbate 20, NF, FCC, (polyoxyethylene 20 sorbitan monolaurate) (TWEEN 20); polysorbate 21 (polyoxyethylene 4 sorbitan monolaurate (TWEEN 21); polyoxyethylene 80 sorbitan monolaurate solution (TWEEN 22 Liquid); polysorbate 40, NF, (polyoxyethylene 20 sorbitan monopalmitate) (TWEEN 40); polysorbate 60, NF, (polyoxyethylene 20 sorbitan monostearate) (TWEEN 60, 60K); polysorbate 61 (polyoxethylene 4 sorbitan monostearate) (TWEEN 61); polysorbate 65 (polyoxethylene 20 sorbitan instearate) (TWEEN 65); polysorbate 80, NF, (polyoxethylene 20 sorbitan monooleate) (TWEEN 80, 80K); polysorbate 81 (polyoxethylene 5 sorbitan monooleate) (TWEEN 81), all available from ICI Americas, Inc. (Bridgewater, N.J.).

iii. Preservatives

For administration to the skin of a human or other mammal, the treatment compositions will often be sterilized or formulated to contain one or more preservatives for incorporation into pharmaceutical, cosmetic or veterinary formulations. These treatment compositions can be sterilized by conventional, well-known sterilization techniques, e.g., boiling or pasteurization when the drug is thermally stable. For drugs that are not thermally stable, then irradiation and/or a preservative may be utilized to provide a sterile composition.

A preservative may be incorporated into a formulation of the present invention in an amount effective for inhibiting the growth of microbes, such as bacteria, yeast and molds. Any conventional preservative against microbial growth can be employed so long as it is pharmaceutically acceptable, is unreactive with the drug(s) contained in the formulation, and is non-irritating or non-sensitizing to human skin. Exemplary preservatives include antimicrobial aromatic alcohols, such as benzyl alcohol, phenoxyethanol, phenethyl alcohol, and the like, and esters of parahydroxybenzoic acid commonly referred to as paraben compounds, such as methyl, ethyl, propyl, and butyl esters of parahydroxybenzoic acid and the like. The amount of preservative is typically not more than about two weight percent, based on the total weight of the formulation.

iv. Colorant

The described compositions may include one or more coloring agents, including components referred to as dyestuffs, which will be present in an effective amount to impart observable coloration to the composition. Opacifiers may also be used, such as zinc oxide. Examples of coloring agents include dyes suitable for food such as those known as F. D. & C. dyes and natural coloring agents such as grape skin extract, beet red powder, beta carotene, annato, carmine, turmeric, paprika, and so forth. The purpose of the colorant may be to impart a pleasing appearance to the composition or to improve its visibility or opacity.

v. pH Adjusters

The compositions may contain pharmaceutically acceptable auxiliary substances as required to approximate physiological conditions and as necessary to prepare compositions for convenient administration, such as pH adjusting and buffering agents. Actual methods for preparing pharmaceutically administrable compounds will be known or apparent to those skilled in the art and are described in detail in, for example, Remington's Pharmaceutical Science.

vi. Other Additives

The described compositions may include one or more additives, such as, for example, fragrances, including pharmaceutically acceptable perfumes; excipients for providing texture (e.g., abrasives or microabrasives); and excipients for providing a cooling or heating sensation (e.g., camphor). Other agents may be incorporated to adjust the isotonic strength of a composition, in particular liquid or semi-solid compositions, of those in particular ones containing water. Tonicity may be adjusted by the inclusion of buffer salts, sodium chloride, or non-ionic species such as dextrose.

### Methods of Making Block Copolymers Compositions and Formulations

In one aspect, the present invention provides a block copolymer composition suitable for drug delivery that includes a drug, a blend of a biodegradable, biocompatible block copolymer and optionally a non-polymeric additive. Typically, the block copolymer composition is non-thermoreversible. In certain embodiments, the block copolymer composition is a liquid in the temperature range of about 20° C. to 42° C. In other embodiments, the block copolymer composition is a semi-solid in the temperature range of about 20° C. to 42° C.

The copolymer can be optionally mixed with a non-polymeric additive in order to modulate the physical and thermal properties of the overall composition. For example, a low molecular weight oligomer, such as PEG 400 can be added to reduce the viscosity of a block copolymer. The blending of the additive to an otherwise viscous block co-polymer therefore renders the overall composition more fluid and enables injection through a syringe/needle assembly.

The block copolymer composition of the present invention may comprise two phases, wherein the first phase comprises (1) block copolymer having hydrophilic blocks and hydrophobic blocks, (2) an optional non-polymeric additive and (3) a bioactive agent, and the second phase comprises a liquid, e.g., water. In further embodiments, the second phase may comprise a carrier, as defined herein.

### A. Synthesis of Triblock Copolymer

In one aspect, the present invention provides a triblock copolymer of the formula ABA. In one embodiment, the A block is hydrophobic, the B block is hydrophilic. Preferably, the ABA triblock copolymers of the invention have a polyalkylene oxide block in the middle (the B block) and two polyester blocks at the ends (the A blocks). Each A and B blocks can themselves be block copolymers. Examples of polyalkylene oxide include polyethylene glycol and PLURONIC® CDC triblock copolymers from BASF (Parsipanny, N.J.). In the structure CDC, C and D are selected from homopolymers of ethylene oxide and propylene oxide. In certain embodiments of the invention, C is a homopolymer of ethylene oxide and D is a homopolymer of propylene oxide, while in another embodiment, C is a homopolymer of propylene oxide and D is a homopolymer of ethylene oxide. Examples of the polyester include PLA, PGA, PCL, Poly(trimethylene carbonate) and copolymers formed from the corresponding monomers such as lactide acid, glycolic acid, TMC, etc.

General methods for making ABA triblock copolymers are provided, for example, by Kimura et al., Polymer 30:1342, 1989. Methods for synthesizing triblock copolymers comprising poly(ε-caprolactone) and polyethylene glycol are described, for example, by Martini et al., J. Chem. Soc. Faraday Trans. 90:1961, 1994. Moreover, methods for diblock polymer synthesis are described, for example, by Zhang et al., Anticancer Drugs 8:696 (1997), and by Ramaswamy et al., J. Pharm. Sci. 86:460 (1997).

In one embodiment, the polyester is a poly(α-hydroxy acid), such as poly(glycolic acid) or poly(lactic acid), which is hydrolyzed in vivo to its constituent α-hydroxy acids and excreted. In one embodiment, for example, the ABA triblock copolymer comprises poly(lactic acid) as the A block and polyethylene glycol as the B block. Preferably, the A and B blocks of such a copolymer are bonded to each other via caprolactone links. An advantage of incorporating caprolactone links is that the resultant triblock copolymer has a fast rate of degradation in vivo. One preferred triblock copolymer of this type can be represented by the structure [poly(DL-lactide-co-ε-caprolactone)]-[polyethylene glycol]-[poly(DL-lactide-co-ε-caprolactone)]. Another preferred triblock copolymer of this type can be represented by the structure poly(DL-lactide-co-glycolide)-[polyethylene glycol]-poly(DL-lactide-co-glycolide).

In another aspect, the present invention provides a triblock copolymer of the formula ABA, wherein A is a diblock including residues having the structure resulting from the polymerization of monomers selected from a cyclic carbonate and glycolide. In one embodiment, the hydrophobic A block has about a 90:10 mole ratio of trimethylene carbonate (TMC) and glycolide (Gly) residues, and B block includes residues having the structure resulting from the polymerization of alkylene oxide. The copolymer thus formed is a liquid at a temperature within the range of 20° C.-42° C.

As used herein, “residues having the structure resulting from the polymerization of” specified monomers refers to the result of the polymerization of those specified chemicals. The same structure may be produced by the polymerization of other monomers and still fall within the scope of the present invention. For instance, a residue of hydroxyacetic acid (HO—CH₂—C(═O)OH) refers to the atoms remaining after hydroxyacetic acid has undergone a homopolymerization reaction so as to form a polyester. In the case of hydroxyacetic acid, such a residue will have the formula —O—CH₂—C(═O)—. In the case of the alkylene oxide, the residue will be an alkylene group joined to an oxygen atom, i.e., —O-alkylene-.

The residue may be formed from the reaction of the specified monomer, or any other monomer, which, upon polymerization, affords the same structure. For instance, any of hydroxyacetic acid, the cyclic diester thereof which is commonly referred to as glycolide, a polyester of the formula (—O—CH₂—C(═O)—)ₙ wherein “n” designates the number of repeating units, or a reactive version of hydroxyacetic acid, e.g., hydroxyacetyl chloride, may be used to form the same residue in the A block of the ABA copolymer of the invention.

Typically, the B block has a number average molecular weight of less than or equal to 2000. In various embodiments, the B block has a number average molecular weight of less than or equal to 1500; less than or equal to 1000; less than or equal to 500; less than or equal to 300. Typically, the copolymer has a number average molecular weight of the B block of at least 200.

Typically, the A block has a number average molecular weight of less than or equal to 2000. In various embodiments, the B block has a number average molecular weight of less than or equal to 1500; less than or equal to 100; less than or equal to 500; less than or equal to 300. Typically, the copolymer has a number average molecular weight of the A block of at least 100. More typically, the copolymer has a number average molecular weight of the A block of at least 500.

In one embodiment, the B block provides 10-50% of the weight of the copolymer, while in other embodiments the B block provides 20-40% of the weight of the copolymer, or 25-35% of the weight of the copolymer.

In a preferred embodiment, at least 50% of the ABA or water-insoluble copolymer is biodegradable. In various embodiments, at least 75% of the copolymer is biodegradable, or at least 90% of the copolymer is biodegradable, or essentially all of the copolymer is biodegradable. Preferably, at least 50% of the A block is biodegradable. In various embodiments, at least 75% of the A block is biodegradable; or at least 90% of the A block is biodegradable; or essentially all of the A block is biodegradable.

### B. Formulations

In some embodiments, the composition may be used directly for a therapeutic purpose while in other it may be used with further manipulation or processing. Thus, inventive compositions include precursors to final formulations or compositions. These precursors include manufacturing intermediates, materials for constitution, materials for dilution, components or a kit intended to be used together. Other components of a final composition are also possible, for example, a particulate composition may be suspended within a second composition to provide a gel or liquid suspension of particles. The composition of the present invention does not, however, form micelles in an aqueous medium or in bodily fluid.

In one aspect, the block copolymers compositions of the invention can be made as a injectable liquid or spreadable cream due to low viscosity and balanced hydrophilicity. Their degradation rate and drug delivery release rate can also be tailored by proper selection of molecular weight and chemical composition.

In certain embodiments, the block copolymer composition can be used directly in the form of an injectable gel, formulated by mixing a triblock copolymer with a non-polymeric additive, such as an oligomer. In one embodiment, the oligomer is a low molecular weight PEG. Examples of the oligomer include PEG 200, PEG 300 and PEG 400. The triblock copolymer is for example PEG400/TMC-Gly(90/10)900. The triblock copolymer can be present in the PEG additive at a w/w concentration of 2.5%, 5%, 10%, 20%, 33% and 50%.

In other embodiments, the block copolymer composition can be formulated into cream or lotion in the presence of a liquid second phase, such as water. Advantageously for this type of formulation, no conventional cream base, such as mineral oil, is required. The cream thus formed can be injectable or spreadable.

In particular, a first phase is formed by combining a triblock copolymer having hydrophobic and hydrophilic blocks, a non-polymeric additive such as a surfactant, and a bioactive agent. The first phase is typically insoluble in water. The first phase, also referred as the oil phase, can then be mixed with water at an elevated temperature, e.g., 75° C., to form an o/w (oil in water) dispersion. The dispersion forms a stable cream upon cooling to room temperature. A thinner cream, i.e., a lotion can be similarly formulated by reducing the amount of the oil phase in relation to the water phase.

Typically, the oil phase is uniformly distributed in the water phase in the form of droplets or particles. The sizes of the droplets are advantageously at sub-micro level, which are suitable for encapsulating a bioactive agent, e.g., a hydrophobic drug. In certain embodiments, the particles have a surface weighted mean diameter of between about 100 nm to about 700 nm, or a volume weighted mean diameter of between about 100 nm to about 1500 nm

In another aspect, the composition of the present invention comprises a carrier. In particular, the composition comprises two phases wherein a first phase comprises triblock copolymer having hydrophobic and hydrophilic blocks, an optional non-polymeric additive, and a bioactive agent, a second phase comprises a carrier. The block copolymer may be dispersed throughout the carrier or may be contained in only certain regions of the carrier, for example, being contained inside a capsule or as a surface coating. The carrier may be a solid, a semi-solid or a liquid. Examples of carriers include, for example, gels, hydrogels, suspension mediums, capsules, tablets, powders, inserts (e.g., vaginal inserts), suppositories, pastes, putties, waxes, creams, sprays, and ointments. In certain embodiments of the invention, the carrier provides for delivery of a bioactive agent (drug), or facilitates administration.

Suitable carrier includes a second polymer, which can be a copolymer or homopolymer. The second polymer may be incorporated in order to achieve or modify certain properties of the formulation such as viscosity, texture, drug release, bioadhesion or other properties described herein to be affected by polymers. For example the polymer may be a polysaccharide, such as a cellulose, chitosan, hyaluronic acid or it may be a polyacrylic acid polymer. In particular, charged polymers are particularly useful in imparting bioadhesion to the composition. In certain embodiments the polymer may be a polyether such as polyethylene glycol or polypropylene glycol, including crosslinked polyethers or co-polymers of polyethers, including PLURONIC®, PLURONIC-R or Tetronic® polymers. In these compositions, the copolymer, for example a triblock copolymer may comprise are very low or very high proportion of the composition, depending on the intended use. Thus in certain embodiments, the copolymer comprises not more than 10% w/w while the second polymer comprises at least 50% w/w. In other embodiments, the reverse is true, and the copolymer comprises greater than 50% w/w while the second polymer comprises less than 10% w/w. In yet other embodiments, the copolymer may comprise only greater than 40, 30, or 20% w/w. The composition may further comprise water, in order to form a gel with a polysaccharide or other water soluble polymer. In these composition, the copolymer may be selected to be one that is 100% w/w water soluble, partly water soluble (e.g. having a weight fraction between 10-100% w/w that is water soluble), or may be substantially water insoluble. This selection is dependent on the intended use or desired properties of the formulation.

Suitable carriers for forming compositions comprising block copolymers are described in further detail below.

i. Gels and Hydrogels

A gel is a semisolid characterized by relatively high yield values as described in Martin's Physical Pharmacy (Fourth Edition, Alfred Martin, Lea and Febiger, Philadelphia, 1993, pp 574-575). Gels possess properties such as elevated viscosity and elasticity, which may be reduced with increased dilution with an aqueous medium such as water. Gels may contain only non-crosslinked and/or partially crosslinked polymers. Alternately, polymers may be crosslinked to form systems that are herein defined as hydrogels, (see, e.g., Goodell et al., Am. J. Hosp. Pharm. 43:1454-1461, 1986; Langer et al., “Controlled release of macromolecules from polymers”, in Biomedical Polymers, Polymeric Materials and Pharmaceuticals for Biomedical Use, Goldberg, E. P., Nakagim, A. (eds.) Academic Press, pp. 113-137, 1980; Rhine et al., J. Pharm. Sci. 69:265-270, 1980; Brown et al., J. Pharm. Sci. 72:1181-1185, 1983; and Bawa et al, J. Controlled Release 1:259-267, 1985). A hydrogel will maintain an elevated level of viscosity and elasticity when diluted with an aqueous solution, such as water. Crosslinking may be accomplished by several means including covalent, hydrogen, ionic, hydrophobic, chelation complexation, and the like. Gels may contain non-crosslinked, fully crosslinked, and partially crosslinked materials.

In certain embodiments of the instant invention, the carrier gel may include a polypeptide or polysaccharide. In some aspects, the polysaccharides and polypeptides of the instant invention can be fashioned to exhibit a variety of forms with desired release characteristics and/or with specific desired properties. For example, polymers can be formed into gels by dispersing them into a solvent such as water. In certain embodiments, polysaccharides and polypeptides and other polymers can be fashioned to release a therapeutic agent upon exposure to a specific triggering event such as pH (see, e.g., Heller et al., “Chemically Self-Regulated Drug Delivery Systems,” in Polymers in Medicine III, Elsevier Science Publishers B.V., Amsterdam, 1988, pp. 175-188; Peppas, “Fundamentals of pH- and Temperature-Sensitive Delivery Systems,” in Gurny et al. (eds.), Pulsatile Drug Delivery, Wissenschaftliche Verlagsgesellschaft mbH, Stuttgart, 1993, pp. 41-55; Doelker, “Cellulose Derivatives,” 1993, in Peppas and Langer (eds.), Biopolymers I, Springer-Verlag, Berlin). Representative examples of pH-sensitive polysaccharides include carboxymethyl cellulose, cellulose acetate trimellitate, hydroxypropylmethylcellulose phthalate, hydroxypropyl-methylcellulose acetate succinate, chitosan, dextran and alginates, sulphated celluloses, such as dextran SO₄.

Likewise, polysaccharides, polypeptides and other polymers can be fashioned to be temperature sensitive (see, e.g., Okano, “Molecular Design of Stimuli-Responsive Hydrogels for Temporal Controlled Drug Delivery,” in Proceed Intern. Symp. Control. Rel. Bioact. Mater. 22:111-112, Controlled Release Society, Inc., 1995; Hoffman et al., “Characterizing Pore Sizes and Water ‘Structure’ in Stimuli-Responsive Hydrogels,” Center for Bioengineering, Univ. of Washington, Seattle, Wash., p. 828; Hoffman, “Thermally Reversible Hydrogels Containing Biologically Active Species,” in Migliaresi et al. (eds.), Polymers in Medicine III, Elsevier Science Publishers B.V., Amsterdam, 1988, pp. 161-167; Hoffman, “Applications of Thermally Reversible Polymers and Hydrogels in Therapeutics and Diagnostics,” in Third International Symposium on Recent Advances in Drug Delivery Systems, Salt Lake City, Utah, Feb. 24-27, 1987, pp. 297-305). Representative examples of thermogelling polymers, such as poly(oxyethylene)-poly(oxypropylene) block copolymers (e.g., PLURONIC®127 from BASF Corporation, Mount Olive, N.J.), and cellulose derivatives. Paclitaxel microspheres having lower, traditional loadings have been incorporated into a thermoreversible gel carrier (WO 00/66085).

Exemplary polysaccharides include, without limitation, hyaluronic acid (HA), also known as hyaluronan, and derivatives thereof (see, e.g., U.S. Pat. Nos. 5,399,351, 5,266,563, 5,246,698, 5,143,724, 5,128,326, 5,099,013, 4,913,743, and 4,713,448), including esters, partial esters and salts of hyaluronic acid. For example, an aqueous solution of HA having a non-inflammatory molecular weight (greater than about 900 kDa) and a concentration of about 10 mg/ml would be in the form of a gel. The aqueous solution may further comprise one or more excipients that serve other functions, such as buffering, anti-microbial stabilization, or prevention of oxidation.

ii. Creams, Lotions and Ointments

Creams, ointments and pastes suitable as carriers in certain embodiments compositions of the invention are conventional delivery systems or cosmetic vehicles. Such formulations are described in texts such as Remington's Pharmaceutical Sciences (17^{th} edition, Alfonso Gennaro, 1985, Mack Publishing Co. Easton Pa.).

Creams, ointment and pastes may be formed from or include absorbent ointment bases (e.g., anhydrous lanolin also called Wool Fat USP XVI; Hydrophilic Petrolatum or hydroxystearin sulphate); oleaginous ointment bases (e.g., Ointment USP XI also called “White Ointment” or “Simple Ointment”, Yellow Ointment, Petroleum Jelly also called “Petrolatum”, or White Petroleum Jelly also called “White Petrolatum”); emulsion bases (e.g., Cold Cream, also called Petrolatum Rose Water Ointment USP XVI, Rose Water Ointment, Hydrophilic Ointment) and also includes precursor thereto or ingredients thereof, including but not limited to, for example, acacia, agar, alginic acid, alginic salts, Bentonite, cross-linked polymers of acrylic acid such as CARBOMER (CarboMer, Inc., San Diego, Calif.), carrageenan, cellulose and derivatives thereof, cholesterol, gelatin, sodium lauryl sulphate, TWEEN (available from ICI Americas, Inc., Bridgewater, N.J., under the trade designation TWEEN) and SPANs, which are sorbitan esters available from ICI Americas, Inc. including SPAN 20 (sorbitan laurate), SPAN 60 (sorbitan stearate), SPAN 80 (sorbitan oleate), BRIJ surfactants, stearyl alcohol, xanthan gum, mucillages, waxes such as paraffin, beeswax, or spermaceti, polyethylene glycol ointment base, petrolatum, oleic acid, olive oil, mineral oil.

iii. Tablets and Capsules

In certain embodiments, the block copolymer compositions can be combined with a carrier to form a tablet. Tablets may be formed by a number of means and using a number of ingredients known to those skilled in the art, and described in texts such as Remington's Pharmaceutical Sciences (17^{th} edition A. Gennaro ed., Mack Publishing Company 1985, Easton Pa., pp 1605-25). Tablets in these embodiments may be designed to be administered by chewing, swallowing, dissolving under the tongue, injection or insertion into a body cavity. Depending on the application, tablets will therefore be designed having definitive physical properties such as disintegration rate, dissolution rate, friability, hardness and drug dose. To accomplish the required design a number of excipients may be used such as diluents, (e.g., dicalcium phosphate, calcium sulphate, lactose, cellulose, kaolin, mannitol, sodium chloride, sugar, starch, sorbitol, or inositol), binders (e.g., starch, gelatin, sucrtose, glucose, dextrose, lactose, natural gums such as sodium alginate, synthetic gums such as Veegum, polyethylene glycol, polyvinylpyrrolidone, or ethyl cellulose), lubricants (e.g., talc, magnesium stearate, or hydrogenated vegetable oil), glidants (e.g., talc or silicone dioxide), disintegrants (e.g., starch, celluloses, aligns, gums, crosslinked polymers, Croscarmelose, or Crospovidone), colorants such as FD and C dyes, flavoring agents, effervescing agents such as sodium bicarbonate, or film or sugar coatings. Tablets may be formulated to provide sustained release, or protection from stomach acid. Microparticles may be added at an appropriate step in the preparation of tablets, such as inclusion into granules, by mixing with powders prior to wet or dry granulation, or by blending block copolymer compositions with preexistent granules.

In yet other embodiments, the carrier may be formed as a capsule in which the interior of the capsule contains block copolymer compositions and optionally other excipients and the exterior is formed by a shell formed for example from gelatin. Capsules may be hard or soft, with the flexibility being modulated by the addition of plasticizers into the shell. Suitable plasticizers include glycerin or sorbitol. Capsules may be formed using techniques, ingredients and methods known to those skilled in the art and described in texts such as Remington's Pharmaceutical Sciences (17^{th} edition A. Gennaro ed., Mack Publishing Company 1985, Easton Pa., pp 1625-30).

iv. Suppositories and Inserts

In certain embodiments of the invention, block copolymer compositions are contained within a carrier, which is a suppository or insert intended to deliver the block copolymer compositions into the rectal or vaginal cavities. Such suppositories may be fabricated by conventional means known to those skilled in the art of pharmaceutical compounding. Typically, suppositories will included a solid matrix in which the block copolymers are contained. The solid is comprised of a low melting material such as cocoa butter, certain block copolymers, such as di and triblock copolymers having a molecular weight in the range of 900-3000, in which the hydrophilic block is a polyethylene glycol or polypropylene glycol having a molecular weight in the range of 200-1000 g/mol, mixtures of polyethylene glycol 1000, 4000 and 6000, or glycerinated gelatin so that upon insertion into a body cavity having a temperature of, for example, greater than 32° C., the matrix will melt, releasing the block copolymer compositions. Suppositories or inserts comprising block copolymer compositions may be fabricated by conventional means by forming a liquid by melting the matrix material, mixing in block copolymer compositions and compression molding or melt molding the material to form the final composition. As disclosed in the prior art, microspheres, with lower, traditional loading levels have been incorporated into suppositories. Microspheres comprising indormethacin (50% w/w) and ethylcellulose have been incorporated into a suppository carrier comprising PEGs (Uzunkaya and Bergisadi, Farmaco. 2003(58) 509-12).

v. Sprays

In certain embodiments of the invention, block copolymer compositions are contained within a carrier that is administered as a spray as a result of, for example, aerosol formation, nebulization, suspension of block copolymer compositions in a gas, including air, and ejection of a liquid through a nozzle to form a mist or droplets. In such embodiments, a spray is meant to include the dispersed system being sprayed, as well as precursors thereto. Sprays may be administered using various devices such as for example, inhalers, nebulizers, syringes equipped with a sprayer, or pressurized canisters equipped with atomizers. Sprays may be inhaled, or applied to a surface such as skin, a serosal or mucosal surface, a wound site, a surgical site, the airways or the throat.

In further aspects, the compositions of the present invention may be fashioned in a wide variety of forms and may include a scaffold in addition to a drug loaded composition, and, optionally, in addition to a carrier matrix.

In compositions including a scaffold, block copolymer compositions may be positioned either on, adjacent to or within the scaffold resulting in a solid or semi-solid structure often having a defined geometry.

Suitable scaffolds include metallic medical implants such as stents, screws, pins, plates or artificial joints; fabrics such as gauze; porous matrices such as sponges made of gelatin (e.g., GELFOAM from Amersham Health), or cellulose or derivatives thereof (e.g., SEPRAFILM); biologically derived matrices such as semi-synthetic heart valves from a mammalian source (e.g., porcine source), autologous or synthetic tissue grafts such as skin or bone; orthopedic implants such as those made of biodegradable polymers such as poly(L-lactide); sutures; catheters (e.g., balloon catheters); implants made, e.g., of polyethylene, silicone, ethylene vinyl acetate copolymer, fluorinated polyethylene derivatives (e.g., TEFLON), or a polyurethane; grafts; stent-grafts; hydrogels; tissue sealants, shunts; aneurysm coils; bandages; or implantable brachytherapy devices. The present compositions may include scaffolds in the form of, e.g., rod-shaped devices, pellets, slabs, particulates, films, molds, threads, or hydrogels.

The scaffold may facilitate delivery of the drug to its intended site of action, and at the same time, the scaffold also may provide other therapeutic effects. For example, a stent may be used to deliver a drug to a blood vessel and to open the blood vessel having a reduced lumen size due to atherosclerosis, a suture may be used to deliver a drug to a wound site while at the same time providing for mechanical closure of the wound site, or a skin graft could be used to deliver a drug to a burn while at the same time promoting tissue regeneration. Because of the possibility of a dual therapeutic action of a composition that includes drug loaded block copolymer compositions and a scaffold, certain embodiments of the invention include a drug and a scaffold wherein the drug is intended to have a therapeutic effect which is complementary, additive or synergistic to the therapeutic effect expected to be achieved by the scaffold itself, yielding an improvement over conventional therapy.

vi. Catheters and Balloon Catheters

In certain embodiments of the invention, the composition may include a scaffold which is a catheter designed to deliver a solution, or surgical device into a lumen within the body. Suitable catheters may be intended for use in the cardiovascular system or the genitourinary tract. In certain other embodiments, the catheter may be equipped with a balloon designed to temporarily occlude a lumen and optionally permanently alter the luminal area, such as an angioplasty balloon. Catheters suitable for use as a scaffold may be fabricated of polymers such as silicone, ethylene vinyl acetate, polyurethanes and may comprise other polymers such as polyethylene, or polytetrafluoroethylene or lubricious coating polymers. Numerous suitable catheters are commercially available from a wide variety of vendors including Boston Scientific Corporation (Natick, Mass.), Cordis Corporation (Miami Lakes, Fla.), C.R. Bard Inc. (Murray Hill, N.J.), and Baxter Healthcare Corporation (Deerfield, Ill.).

Stents may be used as a scaffold by positioning high drug loading block copolymer compositions, optionally using a carrier such as a gel or hydrogel, onto the surface of the catheter, or into pores within catheter wall. The block copolymer, and optionally a carrier, may be applied by means such as dipping, spraying or painting a polymeric solution. Optionally, the copolymer may be incorporated at the time of catheter manufacture. In the case of balloon catheters, copolymer could be incorporated into the device such that the balloon is inflated with a carrier containing block copolymer compositions. The balloon catheter may be so constructed as to allow a fluid copolymer matrix to pass through the inflated balloon, being delivered to the lumen wall.

vii. Stents

In certain embodiments of the invention, the composition may comprise a scaffold which is a stent designed to maintain the opening of a lumen within the body.

A wide variety of stents may be developed to contain and/or release the high loading block copolymer compositions provided herein, including esophageal stents, gastrointestinal stents, vascular stents, biliary stents, colonic stents, pancreatic stents, ureteric and urethral stents, lacrimal stents, Eustachian tube stents, fallopian tube stents, nasal stents, sinus stents and tracheal/bronchial stents. Stents that can be used in the present invention include metallic stents, which may be fabricated of materials comprising metals, such as, for example, titanium, nickel, or suitable alloys such as steel or nickel-tatnium, polymeric stents, biodegradable stents and covered stents. Stents may be self-expandable or balloon-expandable, composed of a variety of metal compounds and/or polymeric materials, fabricated in innumerable designs, used in coronary or peripheral vessels, composed of degradable and/or nondegradable components, fully or partially covered with vascular graft materials or “sleeves”, and can be bare metal or drug-eluting.

Stents may be readily obtained from commercial sources, or constructed in accordance with well-known techniques. Representative examples of stents include those described in U.S. Pat. No. 4,768,523, entitled “Hydrogel Adhesive”; U.S. Pat. No. 4,776,337, entitled “Expandable Intraluminal Graft, and Method and Apparatus for Implanting and Expandable Intraluminal Graft”; U.S. Pat. No. 5,041,126 entitled “Endovascular Stent and Delivery System”; U.S. Pat. No. 5,052,998 entitled “Indwelling Stent and Method of Use”; U.S. Pat. No. 5,064,435 entitled “Self-Expanding Prosthesis Having Stable Axial Length”; U.S. Pat. No. 5,089,606, entitled “Water-insoluble Polysaccharide Hydrogel Foam for Medical Applications”; U.S. Pat. No. 5,147,370, entitled “Nitinol Stent for Hollow Body Conduits”; U.S. Pat. No. 5,176,626, entitled “Indwelling Stent”; U.S. Pat. No. 5,213,580, entitled “Biodegradable Polymeric Endoluminal Sealing Process”; and U.S. Pat. No. 5,328,471, entitled “Method and Apparatus for Treatment of Focal Disease in Hollow Tubular Organs and Other Tissue Lumens.” Drug delivery stents are described, e.g., in PCT Publication No. WO 01/01957 and U.S. Pat. Nos. 6,165,210; 6,099,561; 6,071,305; 6,063,101; 5,997,468; 5,980,551; 5,980,566; 5,972,027; 5,968,092; 5,951,586; 5,893,840; 5,891,108; 5,851,231; 5,843,172; 5,837,008; 5,766,237; 5,769,883; 5,735,811; 5,700,286; 5,683,448; 5,679,400; 5,665,115; 5,649,977; 5,637,113; 5,591,227; 5,551,954; 5,545,208; 5,500,013; 5,464,450; 5,419,760; 5,411,550; 5,342,348; 5,286,254; and 5,163,952. Removable drug-eluting stents are described, e.g., in Lambert, T. (1993) J. Am. Coll. Cardiol.: 21: 483A. Moreover, the stent may be adapted to release the desired agent at only the distal ends, or along the entire body of the stent. Self-expanding stents that can be used include the coronary WALLSTENT and the SciMED RADIUS stent from Boston Scientific, Natick, Mass. Examples of balloon expandable stents that can be used include the CROSSFLEX stent, BX-VELOCITY stent and the PALMAZ-SCHATZ Crown and Spiral stents from Cordis, the V-FLEX PLUS stent by Cook, Inc., the NIR and EXPRESS stents by Boston Scientific Corp., the ACS MULTILINK and MULTILINK PENTA stents by Guidant Corp., the Coronary Stent S670 and S7 by Medtronic AVE, and the PAS stent by Progressive Angioplasty Systems Inc. In addition to using the more traditional stents, stents that are specifically designed for drug delivery can be used. Examples of these specialized drug delivery stents as well as traditional stents include those from Conor Medsystems (Palo Alto, Calif.) (U.S. Pat. Nos. 6,527,799; 6,293,967; 6,290,673; 6,241,762; U.S. Patent Application Nos. 2003/0199970 and 2003/0167085; and PCT Publication WO 03/015664). Other types of stents for use as scaffolds include coronary stents such as, for example, AVE Micro stent, FREEDOM stent, or the SciMED self expanding stent. Additional exemplary coronary stents are listed in the Handbook of Coronary Stents (PW Serruys, Mosby, St Louis, 1997). Suitable stents may also be designed or used in peripheral blood vessels, the bile duct (e.g., DYNALINK or OMNILINK from Advanced Cardiovascular Systems, Inc., Santa Clara, Calif.), the duodenum (e.g., WALLSTENT), the esophagus (e.g., WALLSTENT), or the trachea or bronchia (e.g., ULTRAFLEX stent from Boston Scientific Co.).

Stent scaffolds may also include polymers such as polyurethanes or polyethylene (van Berkel et al, Endoscopy 2003(35) 478-82), poly(L-lactide) (Su et al, Ann. Biomed Eng 2003(31) 667-77; Tsuji et al Int. J. Cardiovasc. Intervent 2003(5) 13-6), bioresorbable polymers (Eberhart et al., J Biomater. Sci. Polym. Ed 2003(14) 299-312) or polytetrafluoroethylene (Gyenes et al., Can J Cardiol. 2003(19) 569-71).

Stents may be used as a scaffold by depositing block copolymer compositions having a high loading of drug, optionally using a carrier such as a gel or hydrogel, onto the surface of the stent, into a depression within the stent structure, into gaps between the stent tines, or into holes formed by means such as drilling into the stent surface (as described in, e.g., US 2003/0068355A1). The block copolymer compositions and optional carrier, may be applied to the stent by means such as dipping, spraying or painting.

viii. Grafts and Stent-Grafts

A wide variety of stent grafts may be utilized as a scaffold within the context of the present invention, depending on the site and nature of treatment desired. Stent grafts may be, for example, bifurcated or tube grafts, cylindrical or tapered, self-expandable or balloon-expandable, unibody, or, modular. Moreover, the stent graft may be adapted to release the desired agent at only the distal ends, or along the entire body of the stent graft. The graft portion of the stent may be composed of a textile, polymer, or other suitable material such as biological tissue. Representative examples of suitable graft materials include textiles such as nylon, acylonitrile polymers, such as ORLON from E.I. Du Pont De Nemours and Company, Wilmington, Del., polyester, such as DACRON from E.I. Du Pont De Nemours and Company, Wilmington, Del.), or woven polytetrafluoroethylene (e.g., TEFLON from E.I. Du Pont De Nemours and Company, Wilmington, Del.), and non-textiles such as expanded polytetrafluoroethylene (PTFE). Representative examples of stent grafts, and methods for making and utilizing such grafts are described in more detail in U.S. Pat. Nos. 5,810,870; 5,776,180; 5,755,774; 5,735,892; 5,700,285; 5,723,004; 5,718,973; 5,716,365; 5,713,917; 5,693,087; 5,683,452; 5,683,448; 5,653,747; 5,643,208; 5,639,278; 5,632,772; 5,628,788; 5,591,229; 5,591,195; 5,578,072; 5,578,071; 5,571,173; 5,571,171; 5,522,880; 5,405,377; and 5,360,443.

A stent grafts used as a scaffold in the present invention may be coated with, or otherwise adapted to release an agent which induces adhesion to vessel walls. Such an agent, such as a profibrotic agent, may be contained within a block copolymer matrix, or a composition comprising in some other manner a block copolymer (e.g., a bioactive agent in a block copolymer micelle, suspended in a solid block copolymer, dissolved or suspended in a liquid copolymer) and may be attached to the graft surface for example by, dipping, or painting, or by electrostatic charge and optionally a “glue” or reinforcing layer such as a hydrogel may be added.

Similarly, a wide range of grafts may also be employed as a scaffold. Synthetic grafts are commonly made of expanded TEFLON but other suitable textiles may be used, as listed above for stent grafts. Microparticles may be incorporated into grafts in a manner similar to that disclosed for stent grafts.

ix. Gauze and Bandages

In certain embodiments of the invention, the composition may comprise a scaffold which is a bandage or a fabric, such as a gauze. The gauze or bandage may be so designed as to be useful for covering a wound for example on the skin, or to be used as a packing into a internal wound or to be used as an adjunct in a surgical procedure. Gauze (e.g., a woven or non-woven mesh material) may be formed of materials such as cotton, rayon or polyester fibers. Bandages may include adhesive and non-adhesive bandages. Block copolymer compositions, particularly those containing a bioactive agent, or having physical properties of barrier enhancement, may be incorporated onto the surface of such a scaffold, or into the porous structure (e.g., within the weave) of a gauze.

x. Sutures

In certain embodiments of the invention, the composition may comprise a scaffold which is a suture designed to effect the closure of a wound or incision, or to fix a tissue or medical device or implant in place. Such a suture may be fabricated of materials and by methods known to those skilled in the art. Suitable sutures may comprise for example biodegradable polymers such as poly(glycolide), poly(lactide) or co-polymers thereof. Sutures may be formed comprising materials such as silk or catgut, nylon, or polypropylene. Suitable sutures may be braided or monofilamentous.

xi. Sponges, Pledgets and Implantable Porous Membranes

In certain embodiments of the invention, the composition may comprise a scaffold which is a sponge, pledget or implantable porous membrane so designed as to allow for the ingress of body fluids or tissues after implantation. Such a device may be fabricated of materials and by methods known to those skilled in the art. Such porous materials may be made of materials such as collagen, gelatin (e.g., GELFOAM), HA and derivatives thereof (e.g., SEPRAMESH or SEPRAFILM from Genzyme Corporation, Cambridge, Mass.), and cellulose. In certain embodiments the sponge may be a pledget comprising materials such as cotton, cellulose, gelatin, or TEFLON. Microparticles may be incorporated into a pledget by suspending them in a carrier and soaking the pledget in the suspension, taking up the liquid and the suspended block copolymer compositions. Microparticles may be loaded in this manner immediately prior to use of the composition, or at an earlier time of manufacture. In certain embodiments, the liquid carrier may then be removed by methods such as drying are using pressure to expel the liquid. In certain embodiments, the carrier may be a semi-solid such as a gel or ointment. The pledget may be implanted or used topically or on a wound surface.

xii. Orthopedic Implants

In certain embodiments of the invention, the composition may comprise a scaffold which is an orthopedic implant designed to provide stability or articulation to the skeletal system, including joints. Implants include pins, screws, plates, grafts (including allografts) of, for example, tendons, anchors, total joint replacement devices, such as artificial knees and hips. The orthopedic implant may be fabricated of materials that include metals, such as, for example, titanium, nickel, or suitable alloys such as steel or nickel-titanium. Suitable orthopedic implants may also comprise polymers such as polyurethanes or polyethylene, polycarbonate, polyacrylates (e.g., polymethyl methacrylate), poly(L-lactide) or polytetrafluoroethylene. Orthopedic implants may also include bone implants that include tricalcium phosphate or hydroxyapatite.

Exemplary orthopedic devices which are suitable scaffolds in certain embodiments of this invention are described in The Radiology of Orthopaedic Implants: An Atlas of Techniques and Assessment by Andrew A. Freiberg (Editor), William, M. D. Martel, Mosby Publishing (2001) ISBN 0323002226.

xiii. Films

Within yet other aspects of the invention, the therapeutic compositions of the present invention comprising a scaffold may be formed as a film. Preferably, such films are generally less than 5, 4, 3, 2 or 1 mm thick, more preferably less than 0.75 mm or 0.5 mm thick, and most preferably less than 500 μm. Such films are preferably flexible with a good tensile strength (e.g., greater than 50, preferably greater than 100, and more preferably greater than 150 or 200 N/cm²), good adhesive properties (i.e., readily adheres to moist or wet surfaces), and have controlled permeability.

xiv. Tissue Sealants

As used herein, the term “sealant” refers to a material which decreases or prevents the migration of fluid from or into a surface such as a tissue surface. Sealants are typically formed by the application of precursor molecules to a tissue followed by local polymerization. The same materials may also be used to adhere materials together, either when applied between them and polymerized, or when used to jointly embed materials. Generally, surgical sealants are absorbable materials used primarily to control internal bleeding and to seal tissue.

Sealant material and devices for delivering sealant materials for use in the instant invention are described, e.g., in U.S. Pat. Nos. 6,624,245; 6,534,591; 6,495,127; 6,482,179; 6,458,889; 6,323,278; 6,312,725; 6,280,727; 6,277,394; 6,166,130; 6,110,484; 6,096,309; 6,051,648; 5,874,500; 6,063,061; 5,895,412; 5,900,245; and 6,379,373.

Sealants, which may be combined with one or more drugs contained at least partly in highly loaded block copolymer compositions, include tissue adhesives (e.g., cyanoacryates and cross-linked poly(ethylene glycol)-methylated collagen compositions) and sealants, including commercially available products, such as COSEAL (Cohesion Technologies, Inc., Palo Alto, Calif.), FLOSEAL (Fusion Medical Technologies, Inc., Fremont, Calif.); SPRAYGEL or a variation thereof (Confluent Surgical, Inc., Boston Mass.); and absorbable sealants for use in lung surgery, such as FOCALSEAL (Genzyme BioSurgery, Cambridge, Mass.).

### C. Incorporation of Drug and Drug Releasing Characteristics

A bioactive agent, or a drug is incorporated in all formulations described above. The drug can be hydrophobic and hydrophilic. The drug can be incorporated by mixing with a block copolymer directly, or with a block copolymer in the presence of a non-polymeric additive and/or a carrier. The drug dissolves or suspends within the block copolymer composition. The resultant drug delivery system has the form of a liquid or semi-solid at room temperature. It therefore does not require any pre-injection mixing. If necessary, the system can be sterilized by gamma radiation, and stored for long periods without compromise in properties.

The amount of drug in a polymeric drug delivery system varies according to the particular drug, the desired therapeutic or prophylactic effect, and the desired duration for which the system is to deliver the drug. In general, the upper limit on the amount of drug included in a polymeric drug delivery system is determined by the need to obtain a suitable viscosity for injection, whereas the lower limit of drug is determined by the activity of the drug and the required duration of treatment. Typically, a polymeric drug delivery system can contain a drug from about 2% to about 30% of the total weight of the system. Preferably, a polymeric drug delivery system contains a hydrophobic drug from about 2.5% to about 20% of the total weight of the system, or from about 2.5% to about 15% of the total weight of the system. For example, a hydrophobic drug can be included in a polymeric drug delivery system at a dose that is 2.5%, 5%, 10%, 15%, 20%, 25%, or 30% of the total weight of the system. Any hydrophobic therapeutic agent can be loaded into the polymeric formulation, as described below.

Pharmaceutical formulation can be prepared by loading therapeutic agents into the triblock copolymers and/or the polymeric blends. The loading can be done by mixing drug directly into the copolymer or by co-dissolving both drug and the copolymer in a common organic solvent (e.g., acetonitrile, dichloromethane) followed by solvent removal using evaporation and/or in vacuo. The second approach is preferred for loading paclitaxel into the ABA triblock copolymers since it ensures homogenicity and a composition that affords fast release of paclitaxel.

Any therapeutic agent can be loaded into the ABA triblock copolymers (in contrast to the polymeric blends, which require a hydrophobic drug). Examples of the agents include, without limitation, peptides, proteins, antigens, vaccines, anti-infectives, antibiotics, antimicrobials, antiallergenics, steroids, decongestants, miotics, anticholinergios, sympathomimetics, sedatives, hypnotics, psychic energizers, tranquilizers, analgesics, antimalarials, and antihistamines.

Within certain aspects of the present invention, the therapeutic composition is biocompatible, and releases one or more bioactive agents over a period of several hours (e.g., 1 hour, 2 hours, 4 hours, 8 hours, 12 hours or 24 hours) to days (e.g, 1 day, 2 days, 3 days, 7 days, or 14 days) to months (e.g., 1 month, 2 months, 3 months, 6 months or 12 months). Further, therapeutic compositions of the present invention should preferably be stable for several months and capable of being produced or maintained or both under sterile conditions. Release profiles may be characterized in terms of the initial rate, time for 50%, 90% or 100% drug release, or by appropriate kinetic models such as zero-order, first order, diffusion controlled (e.g., square-root of time, Higuchi model) kinetics, or by the number of distinct phases of release rate (e.g., monophasic, biphasic, or triphasic). The release profile may be characterized by the extent of its burst (initial) phase. The burst phase may result in little or large amounts of drug release and consequently microparticles may be defined as “low” or “high” burst systems. For example, low burst systems may release as little as about 30, 20, 10 or even 5 or 1% of the total amount loaded in the initial phase of release. High burst systems may release at least about 50, 60, 70 or even 100% of the total amount of drug in the burst phase. The duration of the burst phase is dependant on the overall intended duration of the release profile. For microparticles intended to release all of the loaded drug within hours, the burst phase may occur over several minutes (e.g., 1 to 30 minutes). For microparticles intended to release over several days, the burst phase may on the order of hours (e.g., 1 to 24 hours). For microparticles intended to release over several weeks, the burst phase may be from several hours to several days (e.g., 12 hours to 7 days). An exemplary release profile describing a composition's release characteristics may be a low burst, releasing less than 10% in the first 24 hours, followed by a phase of approximately zero-order release and a gradual reduction in rate after 5 days, until all of the drug is depleted. Compositions within the scope of this invention may have a wide range of release characteristics depending on the composition. For example, a mycophenolic acid or 5-fluorouracil loaded microparticle made of a relatively hydrophilic polymer will have a high burst and release all of the drug with in several hours to a few days. Alternately, paclitaxel loaded composition may release only a small fraction of the total dose over 5 days, with a very small burst phase.

In addition to the effect of the initial drug loading to the drug release profile, the duration and rate of release can be further controlled by modulating the ratio of water soluble polymer to water insoluble polymer. Therefore, by careful selection of the ratio of drug:water, and/or soluble polymer:water insoluble polymer, the composition may be tuned to fit the required treatment needs.

Further, therapeutic compositions of the present invention should preferably be stable for several months and capable of being produced or maintained under sterile conditions.

In one embodiment, the drug release from these compositions can be diffusion controlled, erosion controlled or a combination of both mechanisms.

In another embodiment, the drug release can be first-order release, zero-order release or a combination of these orders of release.

The polymeric composition may also be fashioned to have particularly desired release characteristics and/or specific properties. For example, polymers and polymeric carriers may be fashioned to release a therapeutic agent upon exposure to a specific triggering event such as pH as discussed above. Likewise, polymers and polymeric carriers may be fashioned to be temperature sensitive as discussed above.

### Therapeutic Uses of Block Copolymer Composition

The block copolymer composition described herein can be used to deliver either a hydrophobic or (dependent on the drug delivery system) a hydrophilic drug in controlled manner either to a localized site or to the systemic circulation.

Advantageously, the present invention does not require the use of organic solvents for dissolving the drugs during manufacturing nor for solidification of the implant. As used herein, the term “organic solvent” refers to non-polymeric substances, such as aromatic hydrocarbons, esters, ethers, ketones, amines, alcohols, nitrated hydrocarbons, and chlorinated hydrocarbons. For example, solvents that are typically used in polymer drug delivery systems include acetone, ethanol, tetrahydrofuran and pyrrolidones. Since these compounds are not biocompatible, they are not suitable for in vivo injection into delicate areas such as the eye, blood vessels, or the synovial joint.

Because the block copolymer compositions of the present invention are non-thermoreversible, they remain as liquid throughout the temperature range between room to physiological temperature. Accordingly, the formulation does not require thermal modification for injection, and consequently, polymeric compositions can be injected at room temperature through narrow gauge needles without blocking. Nevertheless, lower viscosity and improved injectability may be attained by warming the polymeric formulation to 37° C. prior to injection. This will allow the viscous liquid or semi-solid compositions to be injected through smaller gauge needles for more delicate tissue areas.

A polymeric drug delivery system (containing a blend of water insoluble and water soluble polymer components with a hydrophobic drug(s)) or a drug in combination with an ABA triblock copolymer (in total, referred to as polymeric compositions, or drug delivery systems), can be administered to a subject by intraperitoneal, intraarticular, intraocular, intratumoral, perivascular, subcutaneous, intracranial, or intramuscular injection. Alternatively, the polymeric compositions can be applied to surgically exposed tissue areas by using an open syringe to extrude the polymeric paste at room temperature. For example, a polymeric composition loaded with paclitaxel can be: (a) injected directly into a solid tumor to treat cancer, (b) applied to a tumor resection site to prevent local recurrence, (c) spread on tissues to prevent post-surgical adhesions, (d) applied perivascularly to treat restenosis, and/or (e) injected intra-articularly to treat arthritis.

The polymeric compositions described herein may also be used to fill the cavities of bones. In such orthopedic or dental applications, the hydrophobic component may be a drug such as a corticosteroid. Alternatively, the hydrophobic component may be a pharmacologically inert compound that promotes the solidification process normally provided by a hydrophobic drug.

For purposes of therapy, a polymeric drug delivery system is administered to a subject in a therapeutically effective amount. A polymeric composition is said to be administered in a “therapeutically effective amount” if the amount administered is physiologically significant. An agent is physiologically significant if its presence results in a detectable change in the physiology of a recipient subject.

The polymeric compositions described herein may be used to treat a variety of animals. In particular, the polymeric compositions are useful for the treatment of mammals, including humans. Various uses of the polymeric compositions, including the drug delivery systems, for human therapy are described above. However, the drug delivery system can also be used for veterinary applications, such as for the treatment of tumors in either farm or domestic animals. In addition, the drug delivery system is useful for the treatment of arthritis, since this disease is common in many animals (e.g., dogs), and arthritis noticed by animal owners due to the visible interference of normal gait in arthritic animals. The drug delivery system may also be useful in the veterinary treatment of restenosis or post-surgical adhesions. In general, the choice of drugs for veterinary applications would be the same as the examples described given for human therapy.

Examples of diseases that may be treated with the block copolymer composition of the present invention include cancer, bacterial infections, psoriasis, arthritis and other inflammatory conditions, fungal infections, vascular disease (e.g., restenosis and aneurysms), surgical adhesions, ocular disease and diabetes. The polymeric drug delivery system can be administered to a patient by intraperitoneal, intraarticular, intraocular, intratumoral, perivascular, parenteral, subcutaneous, intracranial or intramuscular injection. In other embodiments, the polymeric drug delivery system can be administered to a patient topically (e.g., to skin). A polymeric drug delivery system may also be administered by application to mucus membranes, including periophthalmic and inside the eyelid, intraoral, intranasal, intrabladder intravaginal, intrauretlral, intrarectal and to the adventitia of an internal organ.

In one embodiment, the present invention provides methods for treating or preventing a wide variety of diseases associated with the obstruction of body passageways, including for example, vascular diseases, neoplastic obstructions, inflammatory diseases, and infectious diseases.

### Treatment of Vascular Diseases

For example, within one aspect of the present invention a wide variety of therapeutic compositions as described herein may be utilized to treat vascular diseases that cause obstruction of the vascular system. Representative examples of such diseases include artherosclerosis of all vessels (around any artery, vein or graft) including, but not restricted to: the coronary arteries, aorta, iliac arteries, carotid arteries, common femoral arteries, superficial femoral arteries, popliteal arteries, and at the site of graft anastomosis; vasospasms (e.g., coronary vasospasms and Raynaud's Disease); restenosis (obstruction of a vessel at the site of a previous intervention such as balloon angioplasty, bypass surgery, stent insertion and graft insertion).

Therapeutic agents and compositions of the present invention may be administered either alone, or in combination with pharmaceutically or physiologically acceptable carrier, excipients or diluents. Generally, such carriers should be nontoxic to recipients at the dosages and concentrations employed. Ordinarily, the preparation of such compositions entails combining the therapeutic agent with buffers, antioxidants such as ascorbic acid, low molecular weight (less than about 10 residues) polypeptides, proteins, amino acids, carbohydrates including glucose, sucrose or dextrins, chelating agents such as EDTA, glutathione and other stabilizers and excipients. Neutral buffered saline or saline mixed with nonspecific serum albumin are exemplary appropriate diluents.

As noted above, therapeutic agents, therapeutic compositions, or pharmaceutical compositions provided herein may be prepared for administration by a variety of different routes, including for example, directly to a body passageway under direct vision (e.g., at the time of surgery or via endoscopic procedures) or via percutaneous drug delivery to the exterior (adventitial) surface of the body passageway (perivascular delivery). Other representative routes of administration include gastroscopy, ECRP and colonoscopy, which do not require full operating procedures and hospitalization, but may require the presence of medical personnel.

Briefly, perivascular drug delivery involves percutaneous administration of localized (often sustained release) therapeutic formulations using a needle or catheter directed via ultrasound, CT, fluoroscopic, MRI or endoscopic guidance to the disease site. Alternatively the procedure can be performed intra-operatively under direct vision or with additional imaging guidance. Such a procedure can also be performed in conjunction with endovascular procedures such as angioplasty, atherectomy, or stenting or in association with an operative arterial procedure such as endarterectomy, vessel or graft repair or graft insertion.

For example, within one embodiment polymeric paclitaxel formulations can be injected into the vascular wall or applied to the adventitial surface allowing drug concentrations to remain highest in regions where biological activity is most needed. This has the potential to reduce local “washout” of the drug that can be accentuated by continuous blood flow over the surface of an endovascular drug delivery device (such as a drug-coated stent). Administration of effective therapeutic agents to the external surface of the vascular tube can reduce obstruction of the tube and reduce the risk of complications associated with intravascular manipulations {such as restenosis (see next), embolization, thrombosis, plaque rupture, and systemic drug toxicity}.

For example, in a patient with narrowing of the superficial femoral artery, balloon angioplasty would be performed in the usual manner (i.e., passing a balloon angioplasty catheter down the artery over a guide wire and inflating the balloon across the lesion). Prior to, at the time of, or after angioplasty, a needle would be inserted through the skin under ultrasound, fluoroscopic, or CT guidance and a therapeutic agent (e.g., paclitaxel impregnated into a slow release polymer) would be infiltrated through the needle or catheter in a circumferential manner directly around the area of narrowing in the artery. This could be performed around any artery, vein or graft, but ideal candidates for this intervention include diseases of the carotid, coronary, iliac, common femoral, superficial femoral and popliteal arteries and at the site of graft anastomosis. Logical venous sites include infiltration around veins in which indwelling catheters are inserted.

The therapeutic agents, therapeutic compositions and pharmaceutical compositions provided herein may be placed within containers, along with packaging material that provides instructions regarding the use of such materials. Generally, such instructions include a tangible expression describing the reagent concentration, as well as within certain embodiments, relative amounts of excipient ingredients or diluents (e.g., water, saline or PBS) which may be necessary to reconstitute the anti-angiogenic factor, anti-angiogenic composition, or pharmaceutical composition.

Treatment of Fibrosis of a Joint including Inflammatory Arthritis

In one embodiment, the invention provides a method of preventing fibrosis in the vicinity of a joint, comprising administering to a patient in need thereof the composition comprising:

(a) a block copolymer comprising one or more blocks A and blocks B, wherein
(i) block B is more hydrophilic than block A,
(ii) the block copolymer has a molecular weight, Mn, of between about 500 g/mol and about 2000 g/mol;

(b) an optional a non-polymeric additive; and

(c) a fibrosis-inhibiting agent

wherein the composition is non-thermoreversible and is a liquid or semi-solid between about 20° C. to about 40° C.

Within certain embodiments of the invention, the composition releases the fibrosis-inhibiting agent that inhibits one or more of the general components of the process of fibrosis (or scarring) associated with joint damage, including: (a) formation of new blood vessels (angiogenesis), (b) migration and/or proliferation of connective tissue cells (such as fibroblasts or synoviocytes), (c) deposition and remodeling of extracellular matrix (ECM) by matrix metalloproteinase activity, (d) inflammatory response by cytokines (such as IL-1, TNFα, FGF, VEGF). By inhibiting one or more of the components of fibrosis (or scarring), joint damage and osteoarthritis development may be reduced or prevented in a previously injured joint.

In one embodiment, the present invention provides a method of treating or preventing inflammatory arthritis. The method comprises administering to a patient in need thereof a composition comprising:

(a) a block copolymer comprising one or more blocks A and blocks B, wherein

(i) block B is more hydrophilic than block A,

(ii) the block copolymer has a molecular weight, Mn, of between about 500 g/mol and about 2000 g/mol;

(b) an optional a non-polymeric additive; and

(c) a fibrosis-inhibiting agent

wherein the composition is non-thermoreversible and is a liquid or semi-solid between about 20° C. to about 40° C.

Inflammatory arthritis is a serious health problem in developed countries, particularly given the increasing number of aged individuals and includes a variety of conditions including, but not limited to, rheumatoid arthritis, systemic lupus erythematosus, systemic sclerosis (scleroderma), mixed connective tissue disease, Sjögren's syndrome, ankylosing spondylitis, Behçet's syndrome, sarcoidosis, and osteoarthritis—all of which feature inflamed and/or painful joints as a prominent symptom.

In one aspect, the present compositions may be used to treat or prevent osteoarthritis (OA). Osteoarthritis is a common, debilitating, costly, and currently incurable disease. The disease is characterized by abnormal functioning of chondrocytes and their terminal differentiation, leading ultimately to the initiation of OA and the breakdown of the cartilage matrix in the articular cartilage of affected joints. Age is the most powerful risk factor for OA, but major joint trauma, excessive weight, and repetitive joint use are also important risk factors for OA. The pattern of joint involvement in OA is also influenced by prior vocational or avocational overload.

OA can be of primary (idiopathic) and secondary types. Primary OA is most commonly related to age. Repetitive use of the joints, particularly the weight-bearing joints such as hips, knees, feet and back, irritates and inflames the joints and causes joint pain and swelling. Eventually, cartilage begins to degenerate by flaking or forming tiny crevasses. In advanced cases, there is a total loss of the cartilage cushion between the bones of the joints. Loss of the cartilage cushion causes friction between the bones, leading to pain and limitation of joint mobility. Inflammation of the cartilage can also stimulate new bone outgrowths (spurs) to form around the joints.

Secondary OA is pathologically indistinguishable from idiopathic OA but is attributable to another disease or condition. Conditions that can lead to secondary OA include obesity, repeated trauma (e.g., ligament tears, cartilage tears), surgery to the joint structures (ligament repairs, menisectomy, cartilage removal), abnormal joints at birth (congenital abnormalities), gout, diabetes, and other metabolic disorders.

In one aspect, the present compositions may be used to treat or prevent rheumatoid arthritis (RA). Rheumatoid arthritis is a multisystem chronic, relapsing, inflammatory disease of unknown cause. Although many organs can be affected, RA is basically a severe form of chronic synovitis that sometimes leads to destruction and ankylosis of affected joints (Robbins Pathological Basis of Disease, by R. S. Cotran, V. Kumar, and S. L. Robbins, W.B. Saunders Co., 1989). Pathologically the disease is characterized by a marked thickening of the synovial membrane which forms villous projections that extend into the joint space, multilayering of the synoviocyte lining (synoviocyte proliferation), infiltration of the synovial membrane with white blood cells (macrophages, lymphocytes, plasma cells, and lymphoid follicles; called an “inflammatory synovitis”), and deposition of fibrin with cellular necrosis within the synovium. The tissue formed as a result of this process is called pannus and eventually the pannus grows to fill the joint space. The pannus develops an extensive network of new blood vessels through the process of angiogenesis which is essential to the evolution of the synovitis. Digestive enzymes (matrix metalloproteinases such as collagenase and stromelysin) and other mediators of the inflammatory process (e.g., hydrogen peroxide, superoxides, lysosomal enzymes, and products of arachadonic acid metabolism) released from the cells of the pannus tissue break down the cartilage matrix and cause progressive destruction of the cartilage. The pannus invades the articular cartilage leading to erosions and fragmentation of the cartilage tissue. Eventually there is erosion of the subchondral bone with fibrous ankylosis and ultimately bony ankylosis, of the involved joint.

It is generally believed, but not conclusively proven, that RA is an autoimmune disease, and that many different arthrogenic stimuli activate the immune response in the immunogenetically susceptible host. Both exogenous infectious agents (Ebstein-Barr virus, rubella virus, cytomegalovirus, herpes virus, human T-cell lymphotropic virus, mycoplasma, and others) and endogenous proteins (collagen, proteoglycans, altered immunoglobulins) have been implicated as the causative agent which triggers an inappropriate host immune response. Regardless of the inciting agent, autoimmunity plays a role in the progression of the disease. In particular, the relevant antigen is ingested by antigen-presenting cells (macrophages or dendritic cells in the synovial membrane), processed, and presented to T lymphocytes. The T cells initiate a cellular immune response and stimulate the proliferation and differentiation of B lymphocytes into plasma cells. The end result is the production of an excessive, inappropriate immune response directed against the host tissues (e.g., antibodies directed against type II collagen, antibodies directed against the Fc portion of autologous IgG (called “Rheumatoid Factor”)). This further amplifies the immune response and hastens the destruction of the cartilage tissue. Once this cascade is initiated, numerous mediators of cartilage destruction are responsible for the progression of rheumatoid arthritis.

In rheumatoid arthritis, articular cartilage is destroyed when it is invaded by pannus tissue (which is composed of inflammatory cells, blood vessels, and connective tissue). Generally, chronic inflammation in itself is insufficient to result in damage to the joint surface, but a permanent deficit is created once fibrovascular tissue digests the cartilage tissue. The abnormal growth of blood vessels and pannus tissue may be inhibited by treatment with fibrosis-inhibiting compositions, or fibrosis-inhibiting agents. Incorporation of a fibrosis-inhibiting agent into these compositions or other intra-articular formulations, can provide an approach that can reduce the rate of progression of the disease.

Thus, within one aspect of the present invention, methods are provided for treating or preventing inflammatory arthritis comprising the step of administering to a patient in need thereof a therapeutically effective amount of a fibrosis-inhibiting agent or a composition comprising a fibrosis-inhibiting agent. Inflammatory arthritis includes a variety of conditions including, but not limited to, rheumatoid arthritis, systemic lupus erythematosus, systemic sclerosis (scleroderma), mixed connective tissue disease, Sjögren's syndrome, ankylosing spondylitis, Behçet's syndrome, sarcoidosis, and osteoarthritis—all of which feature inflamed and/or painful joints as a prominent symptom.

An effective fibrosis-inhibiting therapy for inflammatory arthritis will accomplish one or more of the following: (i) decrease the severity of symptoms (pain, swelling and tenderness of affected joints; morning stiffness, weakness, fatigue, anorexia, weight loss); (ii) decrease the severity of clinical signs of the disease (thickening of the joint capsule, synovial hypertrophy, joint effusion, soft tissue contractures, decreased range of motion, ankylosis and fixed joint deformity); (iii) decrease the extra-articular manifestations of the disease (rheumatic nodules, vasculitis, pulmonary nodules, interstitial fibrosis, pericarditis, episcleritis, iritis, Felty's syndrome, osteoporosis); (iv) increase the frequency and duration of disease remission/symptom-free periods; (v) prevent fixed impairment and disability; and/or (vi) prevent/attenuate chronic progression of the disease.

According to the present invention, any fibrosis-inhibiting agent described above could be utilized in the practice of this invention. Within certain embodiments of the invention, the composition may release an agent that inhibits one or more of the general components of the process of fibrosis (or scarring) associated with inflammatory arthritis, including: (a) formation of new blood vessels (angiogenesis), (b) migration and/or proliferation of connective tissue cells (such as fibroblasts or synoviocytes), (c) destruction of the cartilage matrix by metalloproteinase activity, (d) inflammatory response by cytokines (such as IL-1, TNFα, FGF, VEGF). By inhibiting one or more of the components of fibrosis (or scarring), cartilage loss may be inhibited or reduced.

The composition can be administered in any manner described herein. However, preferred methods of administration include intravenous, oral, subcutaneous injection, or intramuscular injection. A particularly preferred embodiment involves the administration of the fibrosis-inhibiting compound as an intra-articular injection (directly, via arthroscopic or radiologic guidance, or irrigated into the joint as part of an open surgical procedure). The fibrosis-inhibiting agent can be administered as a chronic low dose therapy to prevent disease progression, prolong disease remission, or decrease symptoms in active disease. Alternatively, the therapeutic agent can be administered in higher doses as a “pulse” therapy to induce remission in acutely active disease; such as the acute inflammation that follows a traumatic joint injury (intra-articular fractures, ligament tears, meniscal tears, as described below). The minimum dose capable of achieving these endpoints can be used and can vary according to patient, severity of disease, formulation of the administered agent, potency and/or tolerability of the agent, clearance of the agent from the joint, and route of administration.

In one aspect, the compositions of the present invention may be used for the management of osteoarthritis in animals (e.g., horses). It should be noted that some HA products (notably HYVISC by Boehringer Ingelheim Vetmedica, St. Joseph, Mo.) are used in veterinary applications (typically in horses to treat osteoarthritis and lameness).

Other fibrosis-inhibiting agents that can be present in the composition treat arthritis include corticosteroids. The most common corticosteroids currently used for inflammatory arthritis are methylprednisolone acetate (DEPO-MEDROL, Pharmacia & Upjohn Company, Kalamazoo, Mich.), and triacinolone acetonide (KENALOG, Bristol-Myers Squibb, New York, N.Y.). By adding a fibrosis-inhibiting agent to the intra-articular corticosteroid injection, the intra-articular injection has the added benefit of helping to prevent cartilage breakdown (i.e., it is “chondroprotective”).

Additional examples of fibrosis-inhibiting agents for use in the treatment of inflammatory arthritis include the following: cell cycle inhibitors including (A) anthracyclines (e.g., doxorubicin and mitoxantrone), (B) taxanes (e.g., paclitaxel, TAXOTERE and docetaxel), and (C) podophyllotoxins (e.g., etoposide); (D) immunomodulators (e.g., sirolimus, everolimus, tacrolimus); (E) heat shock protein 90 antagonists (e.g., geldanamycin); (F) HMGCoA reductase inhibitors (e.g., simvastatin); (G) inosine monophosphate dehydrogenase inhibitors (e.g., mycophenolic acid, 1-alpha-25 dihydroxy vitamin D₃); (H) NF kappa B inhibitors (e.g., Bay 11-7082); (I) antimycotic agents (e.g., sulconizole) and (J) p38 MAP kinase inhibitors (e.g., SB202190), as well as analogues and derivatives of the aforementioned.

The drug dose administered from the present compositions for the treatment of inflammatory arthritis will depend on a variety of factors, including the type of formulation and treatment site. However, certain principles can be applied in the application of this art. Drug dose can be calculated as a function of dose per unit area (of the treatment site), total drug dose administered can be measured and appropriate surface concentrations of active drug can be determined. For local application, drugs are to be used at concentrations that range from several times more than to 50%, 20%, 10%, 5%, or even less than 1% of the concentration typically used in a single systemic dose application. In certain aspects, the fibrosis-inhibiting agent is released from the polymer composition in effective concentrations in a time period that may be measured from the time of infiltration into tissue adjacent to the device, which ranges from about less than 1 day to about 180 days. Generally, the release time may also be from about less than 1 day to about 180 days; from about 7 days to about 14 days; from about 14 days to about 28 days; from about 28 days to about 56 days; from about 56 days to about 90 days; from about 90 days to about 180 days. In one aspect, the drug is released in effective concentrations for a period ranging from 1-90 days.

The exemplary fibrosis-inhibiting agents, used alone or in combination, should be administered under the following dosing guidelines. The total amount (dose) of anti-scarring agent in the composition can be in the range of about 0.01 μg-10 μg, or 10 μg-10 mg, or 10 mg-250 mg, or 250 mg-1000 mg, or 1000 mg-2500 mg. The dose (amount) of anti-scarring agent per unit area of surface to which the agent is applied may be in the range of about 0.01 μg/mm²-1 μg/mm², or 1 μg/mm²-10 μg/mm², or 10 μg/mm²-250 μg/mm², 250 μg/mm²-1000 μg/mm², or 1000 μg/mm²-2500 μg/mm².

According to another aspect, any anti-infective agent described above may be used in conjunction with compositions for the treatment of inflammatory arthritis. Exemplary anti-infective agents include (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin), as well as analogues and derivatives of the aforementioned.

### Treatment and Prevention of Cartilage Loss

In another aspect, the polymeric compositions can be used to prevent or reduce the loss of cartilage loss following an injury (e.g., cruciate ligament tear and/or meniscal tear). It has been known for a long time that damage to a joint can predispose a patient to develop osteoarthritis in the joint at a subsequent point in time, but there has been no effective treatment to prevent this occurrence. Instead most of the focus from the medical community and researchers has been on the treatment of the arthritis after it has become established. Treatments for established disease include anti-inflammatory drugs (non-steroidal and steroidal), lubricants or synovial fluid replacements, surgery and joint replacement for severe disease.

Trauma to a joint can take many forms, ranging from a simple sprain which can heal spontaneously to a fracture that creates so many bone fragments that it is almost impossible to reconstruct the joint. The focus for treatment of these injuries revolves around restoring the joint to its normal anatomical state and to resume regular motion. Risk factors for developing arthritis are related to the extent of trauma, the extent of the joint disruption, the degree of the fracture or dislocations, whether or not it is a weight bearing joint, and the characteristic of the joint itself. In general, the greater the trauma to the joint, the greater the risk that the patient will develop osteoarthritis later in life. Surgical correction of a joint to its pre-injury anatomy does not guarantee the prevention of arthritis. In the case of an intra-articular fracture, for example a plateau fracture of the tibia, the treatment is to surgically reconstruct the joint so that it reverts back to a congruent, smooth and intact joint surface with no “step defects” or pieces out of place that would interfere with the gliding of the femur on its surface. Despite improved surgical techniques in repairing these fractures, patients with such fractures have a very high probability of developing degenerative arthritis later on in life.

Anterior cruciate ligament (ACL) injuries in the knee represent a classic example of an injury that predisposes patients to potentially severe degenerative arthritis. The ACL is the ligament that joins the anterior tibial plateau to the posterior femoral intercondylar notch. It is composed of multiple non-parallel fibers with variable fiber lengths that function in bundles to provide tension and mechanical stability to the knee throughout its range of motion. The ACL's stabilizing role has four main functions, including (a) restraining anterior translation of the tibia; (b) preventing hyperextension of the knee; (c) acting as a secondary stabilizer to the valgus stress, reinforcing the medial collateral ligament; and (d) controlling rotation of the tibia on the femur during femoral extensions, and thus, controlling movements such as side-stepping and pivoting. Generally, ACL deficiency results in subluxation of the tibia on the femur causing stretching of the enveloping capsular ligaments and abnormal shear forces on the menisci and on the articular cartilage. Delay in diagnosis and treatment gives rise to increased intra-articular damage as well as stretching of the secondary stabilizing capsular structures.

Despite the known high risk for developing osteoarthritis, patients generally have no associated fractures and have normal x-rays at the time of presentation post-ACL injury. Yet it is well documented that anyone who suffers an ACL injury has a high probability of developing arthritis: 50% by 10 years and 80% by 20 years post-injury. Generally after an ACL rupture patients suffer from instability since the ligament is critical in stabilizing the joint during pivoting and rotation. For example, it is not only required for demanding pivoting sports such as basketball, it is also required for daily activity such as a mother holding her baby as she pivots to get an item from the fridge.

The typical treatment and management of an ACL tear is reconstruction using a graft to replace the torn ACL. The graft may be taken from elsewhere in the patient's extremity (autograft), harvested from a cadaver (allograft) or may be made from a synthetic material. Autograft is the most widely performed orthopedic ACL reconstruction. The technique involves harvesting the patient's own tissue, which may be the mid-third of the patellar tendon with bone attached at both ends, one or two medial hamstrings, or the quadriceps tendon with bone at one end. Synthetic materials have the advantage of being readily available, however, there is a higher failure rate of synthetic grafts compared to autografts and allografts and they have mechanical properties that do not closely resemble the normal ligament. Successful ACL reconstruction is dependent on a number of factors, including surgical technique, post-operative rehabilitation and associated secondary ligament instability. During the surgical procedure, arthroscopy is used to determine whether there are any other associated injuries, which may be treated at the same time, such as meniscal tears or chondral trauma. The surgical procedure is done through a small accessory incision, whereby a tunnel is drilled through the tibia and femur so that the graft may be inserted and fixed.

Surgical reconstruction was initially thought to provide a permanent solution: re-establish a stable knee and prevent degeneration. But other studies demonstrated that after joint injury, there is a cascade of inflammatory activity that once initiated, can be destructive to the joint. This explains why surgical repair itself would have not impact on the prevention of degeneration in traumatized joints; stabilizing a joint or the macro reconstruction of a joint does not address the fundamental underlying biology. Unfortunately, although long-term data has shown that surgery is indeed successful in stabilizing the knee and getting people back to normal activity; it has no impact on the subsequent rate of development of osteoarthritis. As a result, the standard of care to day is to repair the joint acutely and treat the arthritis when it ultimately develops. It should be noted that all joints (in addition to knees) have the potential to become arthritic after trauma, but joints typically involved include; fingers, thumbs, metacarpal (wrist), elbow, shoulder, spine joints (facets, sacro-iliac), temperomandibular, otic bones, hips, ankles, tarsal and toes, especially the hallux.

Fibrosis-inhibiting agents such as paclitaxel have demonstrated in animal experiments an ability to prevent cartilage breakdown following cruciate ligament tears. This effect has been seen both in an inflammatory model and biomechanical model of joint injury. In the inflammatory carrageenin-induced arthritis model in rabbits, paclitaxel demonstrated cartilage. Hartley Guinea pigs subjected to surgical transaction of the anterior cruciate ligament represent a mechanical model for arthritis. Typically after the anterior cruciate is severed, the animals develop arthritis within several weeks. The introduction of the fibrosis-inhibiting agent paclitaxel into the joint greatly retarded the arthritic process and protected not only the cartilage, but also the underlying bone, from breakdown.

The present invention addresses a significant unmet medical need: the prevention of progressive joint degeneration after traumatic injury. Introduction of a composition containing a fibrosis-inhibiting agent into a damaged joint shortly after injury, (e.g., through intra-articular injection, peri-articular administration, via arthroscope, as a joint lavage during open surgical procedures) will impact the cascade of events that lead to joint destruction, such as inhibiting inflammation and preventing cartilage matrix destruction. Most ligament injuries are severe enough or painful enough that patients seek immediate medical attention (within the first 24 to 48 hours); long before irreversible changes have occurred in the joint. If at the time of initial presentation to a health care professional, an intra-articular injection of a fibrosis-inhibitor can be administered into the joint to stop or slow down the destructive activity (in the joint and the tissues surrounding the joint), the articular cartilage can be protected from breakdown. Early introduction of the agents of the present invention intervention will slow, decrease or eliminate the cascade of events that lead to osteoarthritis. The invention can be administered immediately after injury, repeated during the period leading up to stabilization surgery, and/or can be administered after surgery is completed.

Thus, within one aspect of the present invention, methods are provided for treating or preventing cartilage loss, comprising the step of administering to a patient in need thereof a therapeutically effective amount of a composition comprising:

(a) a block copolymer comprising one or more blocks A and blocks B, wherein
(i) block B is more hydrophilic than block A,
(ii) the block copolymer has a molecular weight, Mn, of between about 500 g/mol and about 2000 g/mol;

(b) a non-polymeric additive; and

(c) a bioactive agent,

wherein the composition is non-thermoreversible and is a liquid or semi-solid between about 20° C. to about 40° C.

An effective therapy for cartilage loss will accomplish one or more of the following: (i) decrease the severity of symptoms (pain, swelling and tenderness of affected joints; (ii) decrease the severity of clinical signs of the disease (thickening of the joint capsule, synovial hypertrophy, joint effusion, soft tissue contractures, decreased range of motion, ankylosis and fixed joint deformity); (iii) increase the frequency and duration of disease remission/symptom-free periods; (iv) delay or prevent the onset of clinically significant arthritis in a joint that has previously been injured; and/or (v) prevent or reduce fixed impairment and disability.

### Treatment of Prostate Cancer

In another embodiment, the invention provides a method of treating prostate cancer. Prostate cancer is the most common cancer and the second highest cause of cancer death in men (Carter et al., Prostate, 16:39-48, 1990). Due to increased public awareness and diagnosis of the disease, the reported incidence of prostate cancer continues to rise each year (Scher, Seminars in Oncology, 21:511-513, 1994). Furthermore, with the prospect of the projected aging of the American population, it is likely that even more cases will appear in the future (Colombel et al., Am. J. Pathol., 143:390-400, 1993). Unfortunately, prostate cancer morbidity is reported to be increasing continuously, or is at best leveling off despite earlier detection of the disease (Scher, Seminars in Oncology, 21:511-513, 1994).

For patients presenting with localized prostate tumors, a number of aggressive therapeutic options are available. Some patients require radical prostasectomy, some require aggressive radiotherapy and/or aggressive chemotherapy. A significant portion of patients treated with radiotherapy fail to respond fully with local recurrence of the prostate tumor. Therefore, patients with recurring localized tumors, or patients with localized tumors who are not candidates for aggressive therapy, would benefit from additional local treatment modalities.

Patients with prostate cancer may present in different stages of the disease so that patients in early stages may have localized lesions only, whereas in advanced disease states, patients may also have metastatic disease that, in turn, may be either androgen dependent or androgen independent. Although most patients have androgen dependent metastatic disease, the size of this patient group is dwarfed by the number of men with localized but non-symptomatic disease. At least 30% of men over 50 years of age have histological evidence of localized prostate cancer yet most of these cancers remain undetected or become a problem during the lifetime of these men (Guileyardo et al., J. Natl. Cancer Inst., 65:311-317, 1980; Wasson et al., Arch. Fam. Med., 2:487-493, 1993; Franks, Cancer, 32:1092-1095, 1973). Although routine screening of asymptomatic men will undoubtedly increase the detection of localized tumors it is not known whether early detection will increase survival rates, especially as many physicians advise a “no therapy” approach to patients with localized tumors.

While this approach does little to satisfy the patient who expects an aggressive treatment for the malignancy (Scher, Seminars in Oncology, 21:511-513, 1994), there is justification for not adopting an aggressive treatment regimen since conservative management and delayed hormone therapy treatment of localized tumors has been shown to be as effective a treatment as radical surgical removal of tumors (Chodak et al., N. Engl. J. Med., 330:242-248, 1994; Madson et al., Scand. J. Urol. Nephrol. Suppl., 110:95-100, 1988). Clearly, alternative chemotherapeutic methods are needed for patients with localized prostate cancer to prevent metastatic progression of the disease and to offer the patient a non-invasive treatment of the tumor.

A more rational approach to the administration of a drug for the treatment of localized prostate tumors can be provided by a slow release implant device that could deliver chemotherapeutically relevant doses of a drug to the tumor site. Such a formulation might avoid the systemic toxicity problems associated with repeated treatment regimens. The prostate gland is amenable to local injection (Reft Broading therapy) and thus a single injection of a drug-loaded polymeric paste formulation administered intra-tumorally into human prostate tumors may be efficacious.

At present, there are no effective chemotherapeutic agents for the treatment of prostate cancer, although drugs such as estramustine and vinblastine, which also inhibit microtubule function, have shown some efficacy against prostate cancer both in vitro (Speicher et al., Cancer Res., 52:4433-4440, 1992; Darby et al., Anticancer Res., 16:3647-3652, 1996; Spencer et al., Drugs, 48:794-847, 1994) and in vivo (Spencer et al., Drugs, 48:794-847, 1994; Seidman et al., J. Urol., 147:931-934, 1992; Pienta et al., Cancer, 75:1920-1926, 1995).

Paclitaxel has also been reported to inhibit human prostate cancer cell growth in vitro (Speicher et al., Cancer Res., 52:4433-4440, 1992; Halder et al., Cancer Res., 56:1253-1255, 1992; Darby et al., Anticancer Res., 16:3647-3652, 1996). Moreover, paclitaxel has been shown to have a potent inhibitory effect on angiogenesis (Oktaba et al., AACR 36:454, 1995), a process that has been proposed as a target for the chemotherapeutic treatment of prostate cancer.

Although angiogenesis is associated with tumor growth in all types of cancer, this process may have particular relevance to prostate cancer. Post-mortem studies have shown that up to 30% of all removed prostates have latent prostate cancer (Guileyardo et al., J. Natl. Cancer Inst. 65:311-317, 1980) in which clinically non-apparent carcinomas may be at a prevascular (and slow growing) phase due to the lack of sufficient angiogenic phenotypes in the tumor mass (Furusato et al., Br. J. Cancer 70:1244-1246, 1994). Furthermore, increased angiogenic activity is also associated with metastatic disease in prostate cancer, and it has been suggested that specific inhibition of angiogenesis might inhibit the development of metastasis (Vukanovic et al., The Prostate 26:235-246, 1995). Indeed, a treatment based on the use of the antiangiogenic drug Linomide has been shown to have both antitumor and antimetastatic effects against prostate tumors grown in rats via inhibition of angiogenesis (Vukanovic et al., The Prostate 26:235-246, 1995).

Therefore, with early detection of prostate cancer, the inhibition of angiogenesis may provide an effective “holding” therapy for many patients with localized tumors. Paclitaxel may therefore provide a particularly useful agent in the treatment of prostate cancer via the induction of tumor cell apoptosis and through the inhibition of tumor angiogenesis.

Studies have been conducted to assess the use of biocompatible, biodegradable polymeric pastes for the site-directed delivery of antineoplastic agents such as paclitaxel (Wintemitz et al., Pharm. Res. 13:368-375, 1996) or bis(maltolato)oxovanadium (Jackson et al., Br. J. Cancer 75:1014-1020, 1997). These surgical pastes were originally designed as an adjunct to tumor resection therapy whereby a residual slow release formulation of the drug would be applied to the resection site to prevent tumor regrowth. Such pastes were composed of polycaprolactone blended with methoxypolyethylene glycol and were applied as a viscous molten paste at 56° C., setting to a solid drug-polymer implant at body temperature. However, the paste was very difficult to inject, due to the viscosity of the polymer, and some large tumors failed to respond fully to the drug implant, probably due to the very slow release characteristics of the formulation (Wintemitz et al., Pharm. Res. 13:368-375, 1996). Hence, there was a failure to achieve a chemotherapeutically effective dose. The present invention provides chemotherapeutically effective doses of one or more drugs.

To date, all chemotherapeutic treatments of prostate cancer have palliative goals so that cure has been a rare feature of any trials (Carducci et al., Seminars in Oncology 23(6) Suppl. 14:56-62, 1996). Generally, a strategy of conservative management and delayed hormone therapy is advised for men with localized prostate cancer, especially if the life expectancy of the patient is less than ten years (Chodak et al., N. Engl. J. Med. 330:242-248, 1994). Paclitaxel-loaded polymers can serve in the effective, non-invasive treatment of localized prostate cancer, which offers a cure rather than a holding therapy for patients of all ages with localized prostate cancer.

In addition to prostate cancer, paclitaxel has shown efficacy against advanced breast, ovarian and non-small cell lung cancer (Spencer et al., Drugs, 48:794-847, 1994). Thus, polymeric drug delivery devices containing paclitaxel can also be used to treat these neoplastic conditions.

The polymeric drug delivery systems described herein can be injected through various gauge needles depending on the ratio of insoluble to water soluble polymer. Compositions comprising 40:60 TB:MePEG polymer blends with 15% drug loading, for example, can be injected through 22- or 23-gauge needles at room temperature, allowing access to all body compartments. These injectable properties are not dependent on pre-dissolving the composition in solvents such as N-methyl-pyrrolidone. The present invention, thus generally described, will be understood more readily by reference to the following examples, which are provided by way of illustration and are not intended to be limiting of the present invention.

### EXAMPLES

In the Examples that follow, DL-lactide and glycolide were purchased from PURAC America (Lincolnshire, Ill.; http://www.purac.com). ε-Caprolactone and stannous octoate were purchased from Aldrich and Sigma Chemicals (each in Milwaukee, Wis.), respectively. Poly(ethylene glycols), (PEGs) with number average molecular weights between 200 and 8,000 were purchased from Union Carbide Corp. (Danbury, Conn.; http://www.unioncarbide.com). All other reactants and reagents were obtained from established supply houses, e.g., Sigma-Aldrich (Milwaukee, Wis., http://www.aldrich.sial.com), Fisher Scientific Co. (Hampton, N.H.; http://www.fisher1.com),

The following abbreviations, as used herein, are defined as follows: CL (ε-caprolactone); DLLA (DL-lactide); DSC (Differential Scanning Calorimetry); g (gram, grams); SPE (solid phase extraction); GPC (gel permeation chromatography); NMR (nuclear magnetic resonance); PCL (poly(s-caprolactone); PDLLA (poly-DL-lactide); PE (polyester); PEG (polyethylene glycol); PGA (polyglycolide); PLA (polylactide); PLC (poly(DL-lactide-co-ε-caprolactone); PLGA (poly(lactide-co-glycolide); PTFE (poly(tetrafluoroethylene), and TB (triblock, triblock copolymer); and T_{g}, (glass transition temperature).

### Example 1

### Synthesis of Block Copolymers

PEG and monomer(s) were weighed into 20×150 mm glass test tubes on a top-loading balance and sealed with screw caps. The weights used were weight ratios of their molecular weights. For example, 3.08 g of PEG 400 and 6.92 g of D,L-lactide were used to make 10 g of PEG 400-poly D,L-lactic acid (900). About 400 ml of heavy mineral oil was added into a 2 L beaker and placed on top of a hot plate. The hot plate was connected to a temperature probe which was set at 302° F. (150° C.), with the hot plate set to heat at setting 4 and stir at setting 3. The test tubes were put into the oil bath carefully once the temperature had equilibrated. The test tubes were vortexed after a homogeneous solution was formed and 5 μl/g polymer of stannous 2-ethylhexanoate was added to each tube as a catalyst. The tubes were vortexed and put into the oil bath for 5 hours, during which the tubes were vortexed briefly at 0.5 hours and 1.5 hours. The polymers were poured into glass dishes and were allowed to cool overnight in a fume hood.

Polyester residues of DL-lactide, glycolide, and ε-caprolactone as well as trimethylene carbonate were reacted to form copolymers with various PEG and methoxy-PEG blocks. This process was used to produce many block copolymers. In some batches the tin catalyst content was varied between 0.05 and 0.5% catalyst, most often 0.5% was used and 0.1% was used commonly for diblock copolymer comprising MePEG. In some batches, the scale of synthesis was altered. Accordingly, reaction vessels of different sizes were used, however the same process was followed. By this means various copolymers were synthesized, as shown in Table 1, where component B was polymerized independently with each of components A1, A2, A3, A4, A5 and A6.

TABLE 1
IDENTITY AND MOLECULAR WEIGHT OF POLYESTERS
AND POLYCARBONATES IN SYNTHESIZED COPOLYMERS

| | | | | | | |
|---|---|---|---|---|---|---|
| B | A1 | A2 | A3 | A4 | A5 | A6 |
| PEG/MePEG | PDLLA | PGA | PCL | PLLA | TMC | 90% TMC/ |
| MW | MW | MW | MW | MW | MW | 10% GA MW |
| (g/mol) | (g/mol) | (g/mol) | (g/mol) | (g/mol) | (g/mol) | (g/mol) |

Triblock copolymers

| | | | | | |
|---|---|---|---|---|---|
| PEG 200 | 200, 400, | 200, | 200, | | |
| | 600, 900, | 2000, | 2000, | | |
| | 2000, | 20000 | 20000 | | |
| | 5000, | | | | |
| | 10000, | | | | |
| | 15000, | | | | |
| | 17500, | | | | |
| | 20000, | | | | |
| | 22500, | | | | |
| | 25000, | | | | |
| | 30,000 | | | | |
| PEG 300 | 300, 600, | | | 300, 600, | 300, 600, |
| | 900 | | | 900 | 900 |
| PEG 400 | 200, 400, | | | 300, 600, | 300, 600, |
| | 600, 900, | | | 900 | 900 |
| | 1600, | | | | |
| | 2000 | | | | |
| PEG 600 | 600, | | 600, | | |
| | 8000 | | 8000 | | |
| PEG 900 | 400, 600, | | | | |
| | 900, | | | | |
| | 2000 | | | | |
| PEG 2000 | 200, | 200, | 200, | | |
| | 2000, | 2000, | 2000, | | |
| | 20000 | 20000 | 20000 | | |
| PEG 5000 | 4000, | | | | |
| | 6000, | | | | |
| | 9000 | | | | |
| PEG 8000 | 600, | | 600, | | |
| | 8000 | | 8000 | | |
| PEG | 200, | 200, | 200, | | |
| 20000 | 2000, | 2000, | 2000, | | |
| | 4000, | 20000 | 20000 | | |
| | 6000, | | | | |
| | 9000, | | | | |
| | 20000 | | | | |
| PPG 425 | 300, 400, | | | | 300, 400, |
| | 600, 900 | | | | 600, 900 |
| PG | 300, 400, | | | | 300, 400, |
| | 600, 900 | | | | 600, 900 |

Diblock Copolymers

| | | | | |
|---|---|---|---|---|
| MePEG | 200, | 200 | 200, | |
| 350 | 2000, | | 2000, | |
| | 20000 | | 20000 | |
| MePEG | 200, | 200, | 200, 500, | |
| 750 | 2000, | 2000, | 2000, | |
| | 3000, | 20000 | 20000 | |
| | 20000 | | | |
| MePEG | 200, 857, | 200, | 200, 500, | 4667, |
| 2000 | 1333, | 1333, | 1333, | 8000, |
| | 1636, | 2000, | 2000, | 18000, |
| | 2000, | 20000 | 3000, | 38000 |
| | 2444, | | 8000, | |
| | 4000, | | 20000 | |
| | 6000, | | | |
| | 9000, | | | |
| | 20000 | | | |
| MePEG | 200, | 200, | 200, | 20000, |
| 5000 | 2000, | 2000, | 2000, | 45000, |
| | 2700, | 20000 | 20000 | 95000 |
| | 3333, | | | |
| | 4000, | | | |
| | 6000, | | | |
| | 7500, | | | |
| | 9000, | | | |
| | 20000 | | | |

Other PEG Triblocks with mixed polyester chains:

PEG 400- Poly(D,L Lactic Acid-co-ε-Caprolactone) (900) (80% LA, 20% CL)
PEG 400- PLGA 70 (65% LA, 35% GA)
PEG 400- PLGA 170 (65% LA, 35% GA)
PEG 400- PLGA 200 (65% LA, 35% GA)
PEG 400- PLGA 400 (65% LA, 35% GA)
PEG 400- PLGA 600 (65% LA, 35% GA)
PEG 400- PLGA 900 (65% LA, 35% GA)
PEG 400- PLGA 1600 (65% LA, 35% GA)
PEG 400- PLGA 2000 (65% LA, 35% GA)
MePEG 2000-Poly valerolactone 1333; MePEG 750-Poly valerolactone 500
MePEG 2000-Poly decanolactone 1333
Abbreviations in the table:
PEG = polyethylene glycol; MePEG = methoxy polyethylene glycol; PDLLA - Poly D,L-lactic Acid; PLLA = poly L-lactic acid; PGA = poly glycolic acid; PCL = poly-ε-caprolactone; PLGA = poly(D,L-lactic-co-glycolic acid); PPG = polypropylene glycol; PG = propylene glycol; TMC = trimethylene carbonate; GA = glycolide; LA = D,L-lactide.

### Example 2

### Determination of the Weight Percent of Water Soluble Material in a Polymer

Empty 50 ml plastic centrifuge tubes were tared and 1 g of polymer was weighed accurately into each tube. 10 ml of deionized water was added to each. The tubes were vortexed, transferred to a 37° C. oven overnight and centrifuged at 2500 rpm for 10 minutes the next morning. The supernatant was removed and discarded to eliminate the water soluble component from the polymer. Another 10 ml of water was added and the above process was repeated. The sample was then frozen in the −20° C. freezer and freeze-dried to completely remove the water. The tube was weighed and the percent mass recovery of the sample and the percent water soluble were calculated.

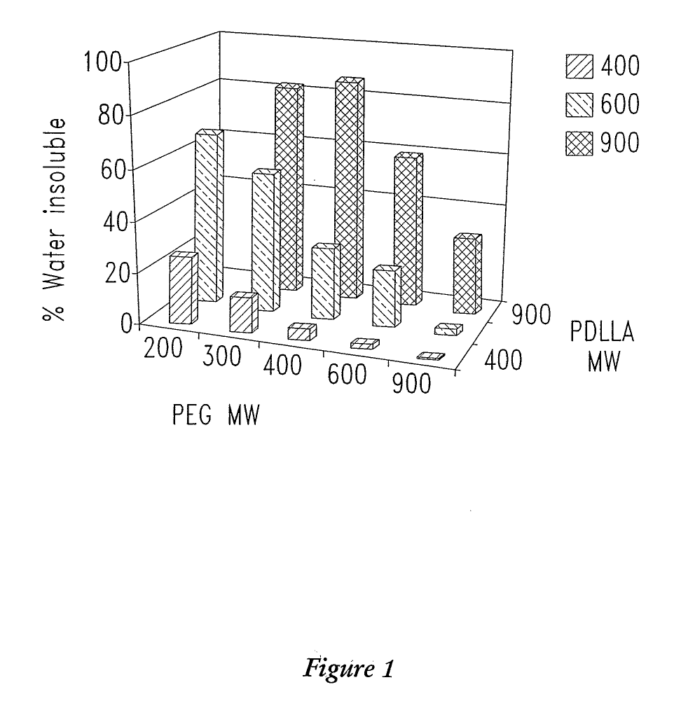

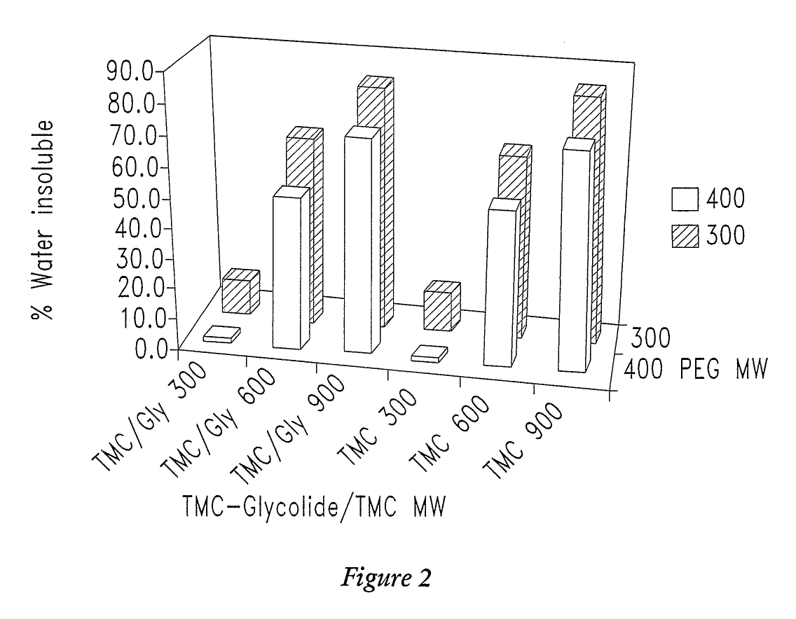

In one experiment, of four polymers tested, all were only partially soluble (25 to 40% dissolved) in water (Table 2). The increased proportion of water soluble component coincided with increasing maximum δh values measured in the solubility screening studies (FIGS. 1 and 2). However, the results were unexpected for PEG400-PLGA900 which was predicted to have a water soluble fraction greater than PEG400-PDLLA900, as the greater density of methyl groups on PDLLA give the polymer more hydrophobic properties than PLGA. The repeatability of this technique was evaluated by testing duplicate samples of PEG400-PDLLA900. The values were nearly identical (Table 2).

GPC data for the polymers were collected before and after the gravimetric study. As seen in Table 2, the number average molecular weight (Mn) increased over 10% (absolute increase of 150-222 g/mol) in all four polymers tested, indicating that the water soluble fractions were the shorter polymer chains in the material. This was expected since shorter chains had proportionally more PEG in the polymer structure, and are thus more hydrophilic.

TABLE 2
WEIGHT RECOVERY OF POLYMERS IN WATER

| | % Water | Mn | Mn | % | Absolute Mn |
|---|---|---|---|---|---|
| Polymer | Soluble | (before) | (after) | Increase | Change |
| PEG400-PLACL | 27.81 | 1172 | 1322 | 12.8 | 150 |
| (900) | | | | | |
| (20% CL, | | | | | |
| 80% LA) | | | | | |
| PEG400- | 24.87 | 1666 | 1837 | 10.3 | 171 |
| (90% TMC, | 24.48 | | | | |
| 10% GA)900 | | | | | |
| PEG400-PDLLA | 39.73 | 1069 | 1232 | 15.2 | 163 |
| (900) | | | | | |
| PEG400-PLGA | 37.29 | 1143 | 1365 | 19.4 | 222 |
| (900) | | | | | |
| (65% LA, | | | | | |
| 35% GA) | | | | | |

A broader range of PEG-PDLLA triblocks were evaluated for percent water soluble fraction in this manner. As the molecular weight of the PEG block in the triblock copolymer increased, the weight percent of polymer recovered after incubation decreased, thus the water soluble fraction increased (FIG. 1). Conversely, as the PDLLA proportion of the triblock copolymer increased, the amount of polymer recovered also increased. PEG 400-PDLLA 900 had greater than 85% water insoluble material in the matrix, while PEG 900-PDLLA 400 was completely water soluble. Thus, by altering the polymer constituents over a relatively narrow range, a wide range of water solubility properties may be achieved. The relationship of a polymer's structure to its mass percent water insoluble fraction when evaluated graphically, as illustrated in FIG. 1, indicates a regular trend which allows prediction of percent water solubility for polymers not tested, but with intermediate polymer molecular weights. Polymers made with 90% mol/mol/10% mol/mol glycolide and 100% TMC [TMC/Gly(90/10)] ranged from nearly completely water soluble (hydrophobic block=300 g/mol) to nearly completely insoluble (hydrophobic block=900 g/mol) (FIG. 2)

### Example 3

### Characterization of the “Max δh” Parameter for a Polymer

The Hansen solubility parameters system was developed by Charles M. Hansen in 1966 for the study of polymer solubility. According to this system, solvents are characterized by three parameters, consisting of a hydrogen bonding component, δh, a polarity component, δp, and a dispersion force component, δd, and all three parameters were related to the total Hildebrand parameter, δt, according to the equation: δt²=δh²+δp²+δd². This system is described in several texts, for example, Hansen Solubility Parameters: A User's Handbook, Charles M Hansen, CRC Press, 2000. For this characterization, solubility parameters were calculated or obtained from data in this text as well as in Handbook of Solubility Parameters and Other Cohesion Parameters, 2^{nd} edition. Allan FM Barton, CRC Press, 1991.

Around 20 mg of polymer was accurately weighed into 20 ml scintillation vials and various solvents or co-solvent mixtures were added in a ratio of 10 mg polymer/ml solvent. The vials were put into a forced air oven at 50° C. overnight, and were allowed to cool to ambient temperature the next morning before making observations. The polymer was considered soluble if there were no visible solids and the solution was clear and transparent. It was very important to check the bottom of the vials as sometimes tiny solid particles were stuck at the bottom of the vial despite having a transparent appearance when viewed from the side. It was also important to note that on some occasions the solids took as long as a few days to come out of solution, especially in xylene and ethoxydiglycol. Polymer solubility was also tested in various solvent blends to assess a wide range of solubility characteristics. The maximum δh value was the highest hydrogen bonding solubility parameter (δh) for any solvent or co-solvent system in which the polymer was soluble at 10 mg/ml. The highest value possible by this method is 42, the δh of water (see, Table 3).

TABLE 3
MAXIMUM δh VALUES OF ALL PEG-PDLLA
TESTED FOR SOLUBILITY

| | | PEG MW | | | | | | |
|---|---|---|---|---|---|---|---|---|
| | | 200 | 400 | 600 | 900 | 2000 | 5000 | 20000 |
| PDLLA | 100 | * | 42 | * | * | * | * | * |
| MW | 200 | 42 | 42 | * | * | 42 | — | 42 |
| | 400 | 32.3 | 42 | * | 42 | * | * | * |
| | 600 | 22.9 | 33 | 36 | * | * | * | * |
| | 900 | 22.9 | 29 | * | 33 | * | * | * |
| | 1600 | * | 15 | * | * | * | * | * |
| | 2000 | 22 | * | * | 23 | 42 | — | 42 |
| | 4000 | * | * | * | * | 15 | 22.3 | 32 |
| | 6000 | * | * | * | * | 15 | 15.2 | 17.3 |
| | 9000 | * | * | * | * | 15.2 | 15.2 | 17.3 |
| | 20000 | 15 | * | * | * | 15 | * | 15 |

*These triblock copolymers were not synthesized

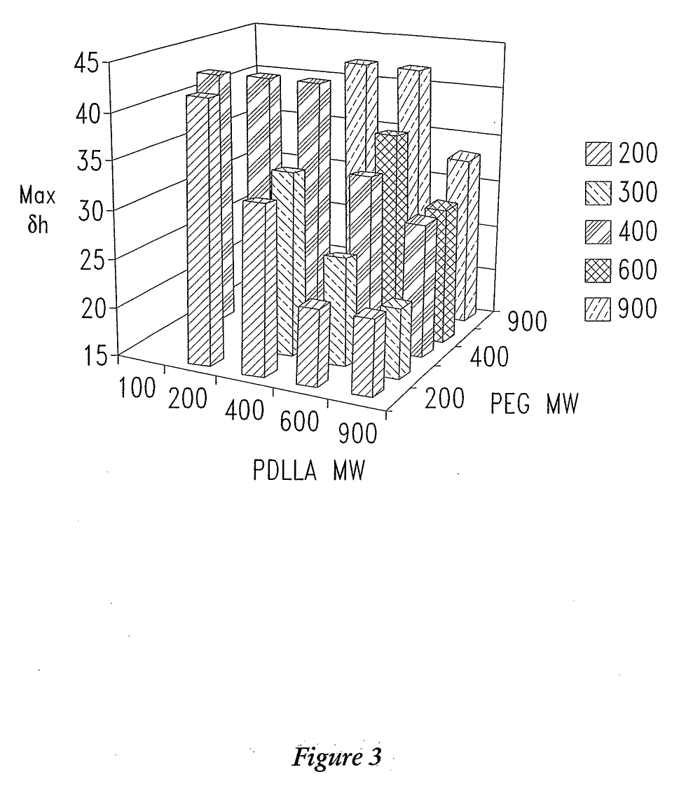

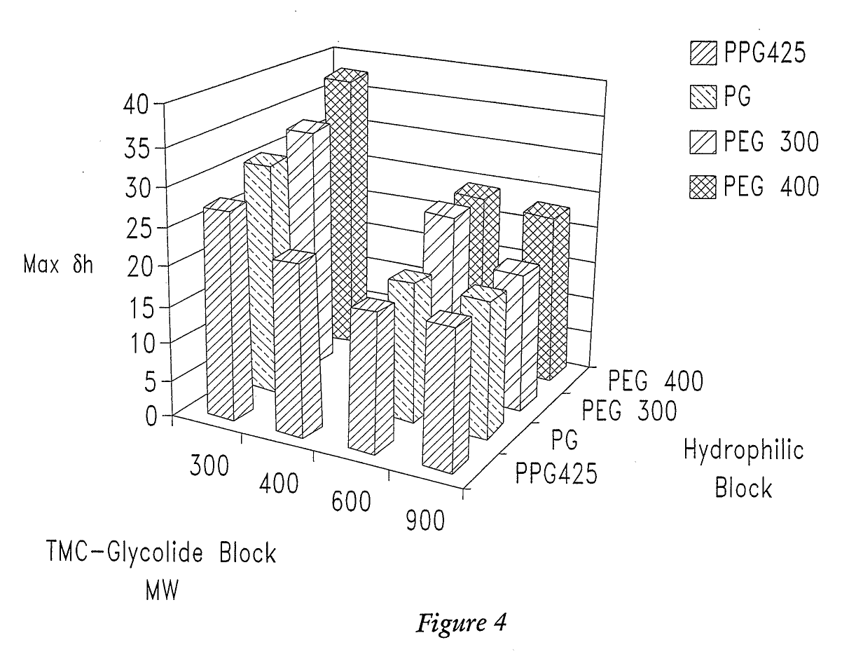

A similar solubility screen for triblock copolymers having polypropylene glycol (PPG) 425 and propylene glycol (PG) as the center hydrophilic block and various hydrophobic block structures: trimethylene carbonate (TMC), trimethylene carbonate-co-glycolide (90/10 mol ratio) (TMC/Gly) and PDLLA. For a given hydrophobic block structure and length PG and PPG 425 resulted in the same max δh for the polymers and PEGs 300 and 400 resulted in similar values as well, although for some polymers (e.g., PEG-TMC/Gly (90/10)), the PEG 400 based polymer had a slightly higher max δh (FIG. 3). Altering the hydrophobic block from 100% TMC to a 90/10 copolymer of TMC and glycolide did not alter the max δh values, yielding a data set shown in FIG. 4.

### Example 4

### Characterization of Drug Release from a Triblock Copolymer Containing Composition

An SPE HPLC method was used to monitor the release characteristics of various block copolymer formulations

### Preparation of Samples for Drug Release Study:

A block copolymer composition loaded a non-ionic, hydrophobic drug (e.g., paclitaxel) was prepared. Around 20 mg of paclitaxel was accurately weighed and dissolved in THF to make a 1 mg/ml solution. Around 4 g of polymer was accurately weighed and 0.5 ml of the paclitaxel solution was added per gram of block copolymer (0.5 mg paclitaxel/gram polymer). The mixture was stirred at 450 rpm inside a 50° C. forced air oven until a homogeneous solution was formed. It was then uncovered and stirred inside the oven for 1 hour. The mixture was transferred into a vacuum oven set at 50° C. and vacuum was applied overnight to remove all the solvent from the polymer.

Drug Release Assay for Paclitaxel loaded Triblock Copolymers:

Approximately 3.5 g of the 0.5 mg/g drug loaded polymer was weighed into a 16×100 mm culture tube (approximately 175 μg of total drug). 11 ml of phosphate buffered saline was dispensed into each tube through a pipette or dispenser and capped. The tubes were placed on a rotating wheel which was set at a 10° incline and rotated at 30 rpm. The apparatus was placed in a 37° C. oven. The sampling time points were at 2, 4 and 7 hours on the first day, daily for the first week and every 48 hours in subsequent weeks. At each sampling time point, the sample was first centrifuged at 2600 rpm for 5 minutes. A 10 ml aliquot was then transferred by glass pipette to a clean 16×100 mm culture tube for solid phase extraction (Table 4). 10 ml of fresh phosphate buffered saline was added to the remaining 1 ml before replacing it on the rotating wheel in the incubation oven. After extraction, the elution solvent (ACN) was dried on a TURBOVAP with N₂ at 35° C. and the solid was reconstituted in 85/15 ACN/water for HPLC analysis.

TABLE 4
SPE METHOD

| Step | Action | Source | Output | Volume | (ml/min) |
|---|---|---|---|---|---|
| 1 | Condition | MeOH | Aq. Waste | 2 | 5 |
| 2 | Condition | H₂O | Aq. Waste | 1.5 | 5 |
| 3 | Condition | Buffer | Aq. Waste | 1 | 5 |
| 4 | Load | Sample | Aq. Waste | 2 | 3 |
| 5 | Load | Sample | Aq. Waste | 2 | 3 |
| 6 | Load | Sample | Aq. Waste | 2 | 3 |
| 7 | Load | Sample | Aq. Waste | 2 | 3 |
| 8 | Load | Sample | Aq. Waste | 2.2 | 3 |
| 9 | Purge-Cannula | ACN | Cannula | 3 | 15 |
| 10 | Rinse | Buffer | Aq. Waste | 3 | 5 |
| 11 | Rinse | H₂O | Aq. Waste | 3 | 5 |
| 12 | Rinse | Vent | Aq. Waste | 6 | 30 |
| 13 | Rinse | Vent | Aq. Waste | 6 | 30 |
| 14 | Collect | ACN | Frac. 1 | 2 | 3 |
| 15 | Purge-Cannula | DCM | Cannula | 6 | 15 |
| 16 | Rinse | DCM | Aq. Waste | 6 | 15 |
| 17 | Purge-Cannula | ACN | Cannula | 6 | 15 |
| 18 | Rinse | ACN | Aq. Waste | 6 | 15 |
| 19 | Purge-Cannula | H₂O | Cannula | 6 | 15 |
| 20 | Rinse | H₂O | Aq. Waste | 6 | 15 |

A triblock copolymer (PEG400/TMC-Gly(90/10)900) having a center hydrophilic block of PEG 400 and two hydrophobic blocks on each end having a combined molecular weight of 900 g/mol and a monomer structure of 90% mol/mol trimethylene carbonate and 10% mol/mol glycolide was dissolved in PEG 300 in various ratios and paclitaxel was added at 0.5 mg/g.

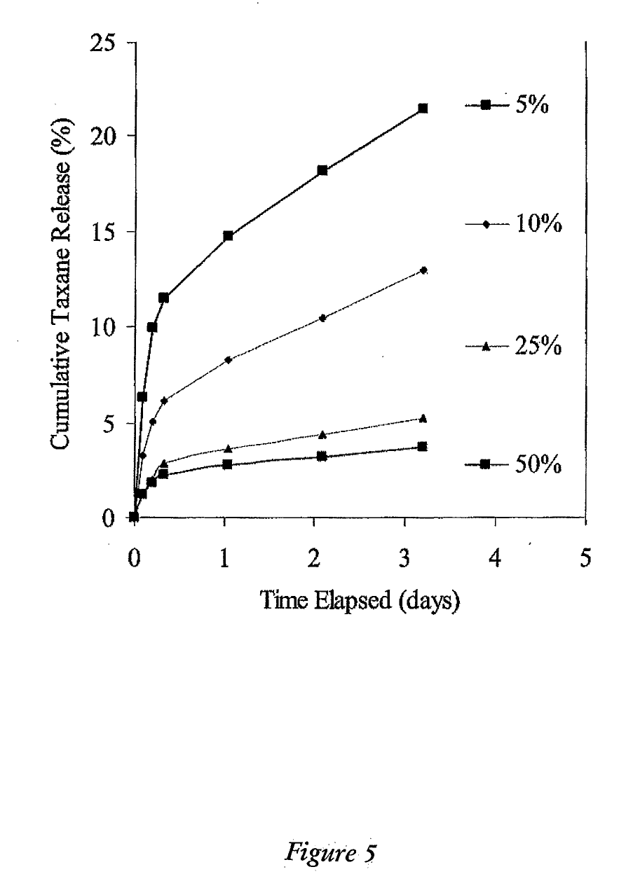

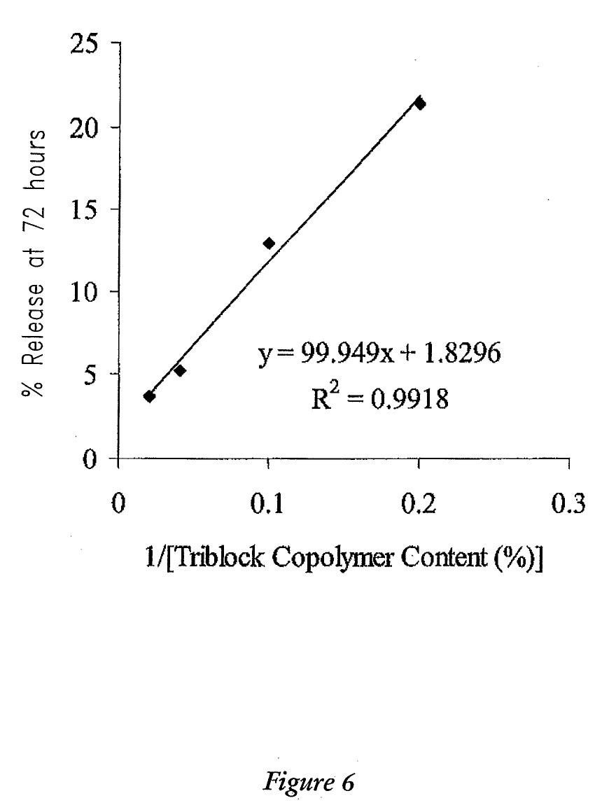

Release study data demonstrate that the compositions provide for highly controlled drug release, having a limited burst phase followed by a linear phase of release. The data are shown in FIG. 5 and FIG. 6 demonstrates the high level of control over release rate by varying the proportion of this triblock copolymer in a paclitaxel formulation.

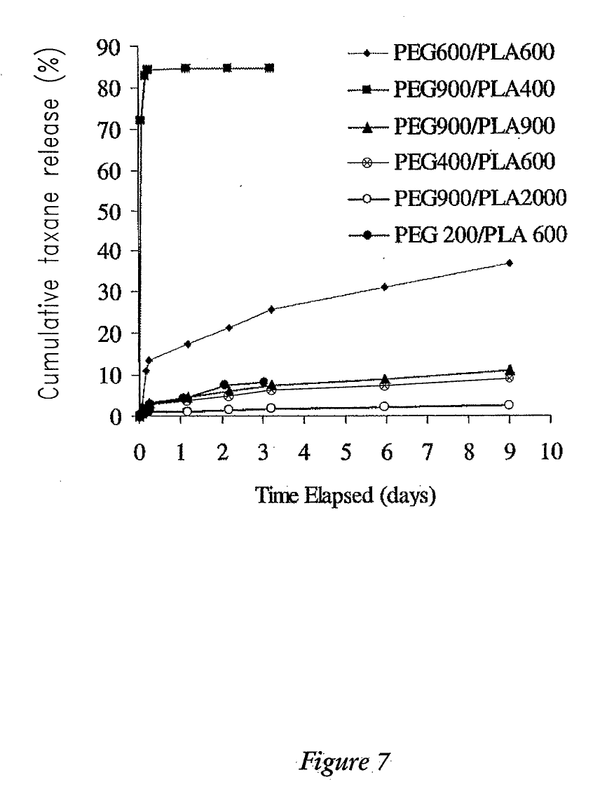

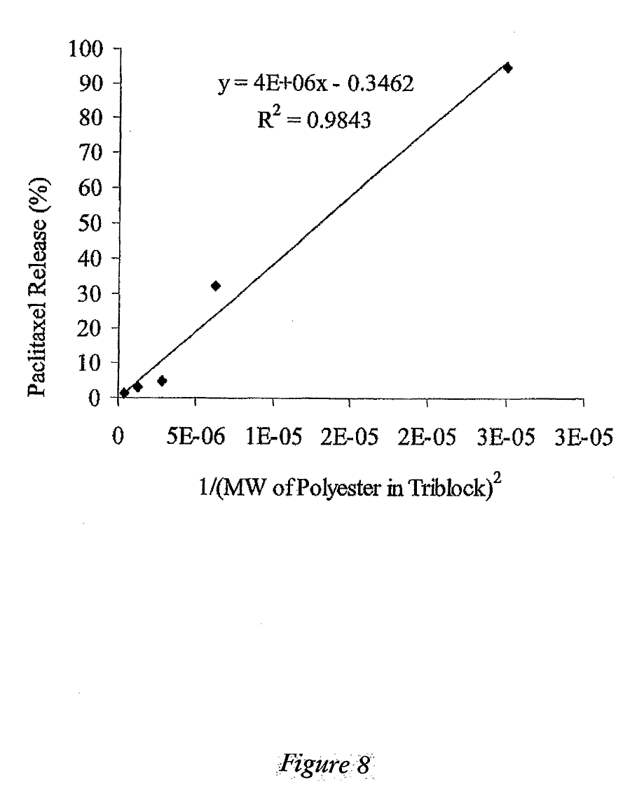

Paclitaxel release characteristics for triblocks having a range of PEG block molecular weights (200 to 900) and PDLLA block total molecular weights (400 to 2000) were evaluated (FIG. 7). In general, as the PDLLA block lengths increased or the PEG block length decreased, the extent of paclitaxel release decreased (FIG. 8). Release ranged from about 85% release in 7 hours from a water soluble copolymer (PEG900/PDLLA400) to only 2% over nine days (PEG900/PDLLA2000). An empirical relationship between extent of release and PDLLA block molecular weight was established. Release after three days was inversely proportional to the square of PDLLA block molecular weight (FIG. 8), indicating that paclitaxel release is very sensitive to the block length of PDLLA.

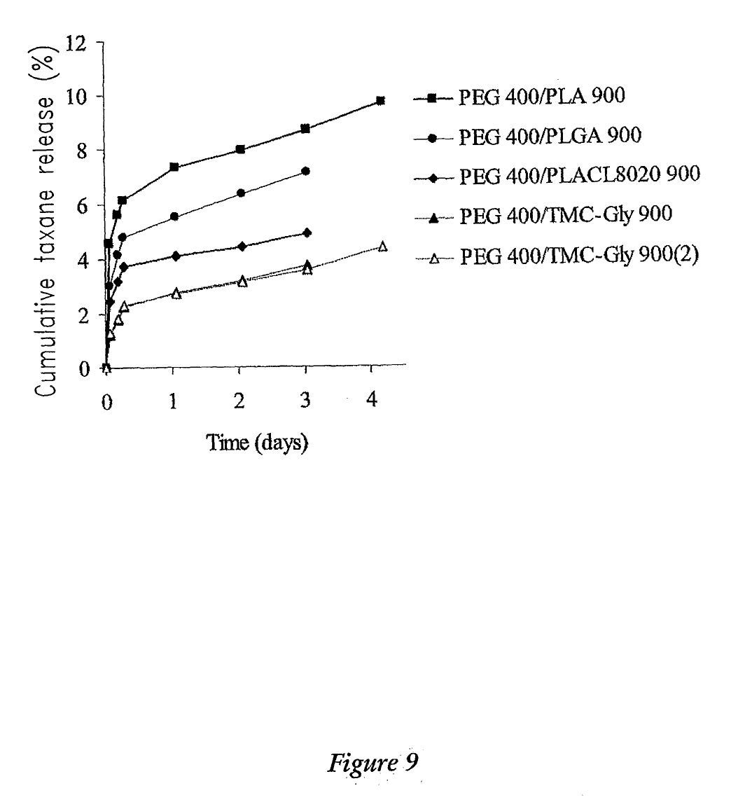

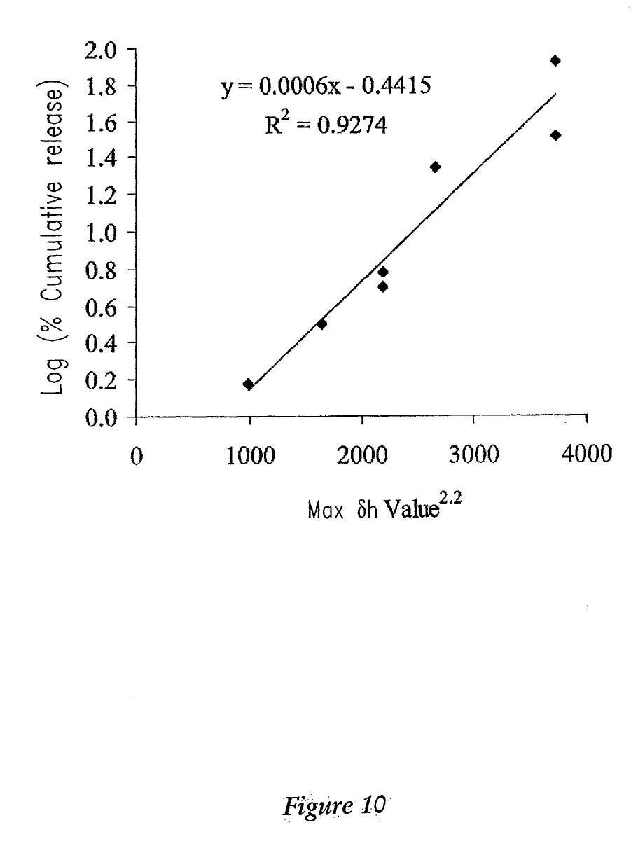

Structural analogues of PEG400/TMC-Gly(90/10)900 (e.g., triblock co-polymers composed of a PEG 400 block and two hydrophobic blocks having a combined molecular weight of 900 g/mol) were analyzed with respect to paclitaxel release characteristics. These data are summarized and compared with release from PEG400/TMC-Gly(90/10)900 in FIG. 9. The analogues were selected for release studies based on their varying solubility characteristics, expressed in maximum δh values determined in earlier solubility screens. Extent of drug release over three days varied with the chemical structure of the hydrophobic blocks in each analogue and an empirical relationship (FIG. 10) relating the extent of release to solubility characteristics was established, also incorporating the data from FIG. 10. The linear regression equation (R²=0.92) relates paclitaxel release to the polymer's maximum δh value, thus in vitro release characteristics may be predicted for all analogues regardless of PEG block molecular weight, hydrophobic block monomer composition and hydrophobic block molecular weight. The relatively simple and rapid solubility screening test can thus be used to rank the performance of all of the polymers in this study and other analogues of this type.

The solubility characteristics of triblock copolymers having a hydrophilic central PEG block can be expressed as the maximum observed δh value at which the polymer was soluble. This parameter was correlated with other polymer characteristics including the percent of water soluble components in the polymer and with paclitaxel release rates from the polymer. An empirical relationship was found to relate polymer solubility characteristics to the extent of paclitaxel release observed over several days.

This release method is also suitable for the characterization of other formulations having a solid or semi-solid component and to monitor the release of other types of bioactive agents.

### Example 5

### Phase Behavior of PEG400-TMC/Gly(90/10)900/PEG 300/Water Mixtures

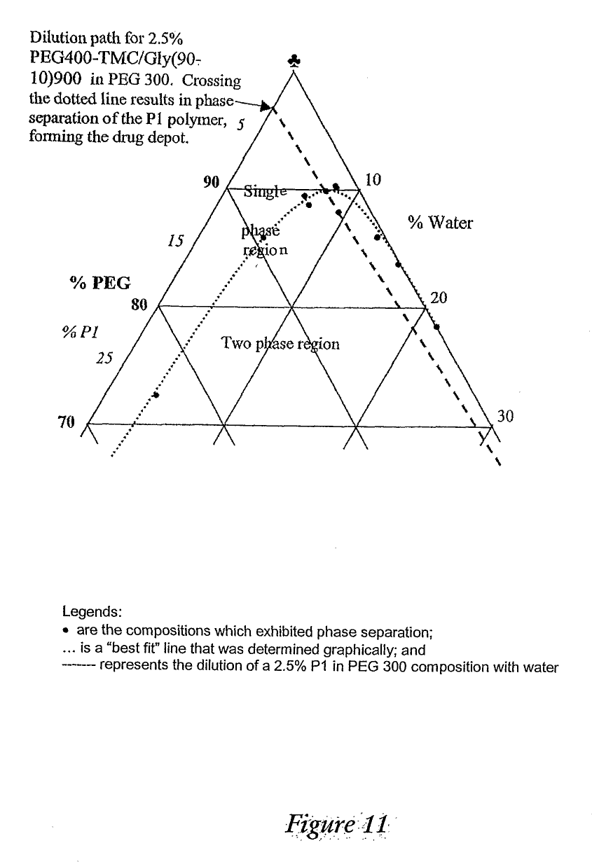

The phase separation of the PEG400-TMC/Gly(90/10)900 triblock copolymer from PEG 300 in the presence of water was evaluated to predict its behavior upon dilution in a largely aqueous physiological environment. The data, represented by a ternary phase diagram (FIG. 11), demonstrate that the mixture containing PEG 300 and the more hydrophobic PEG400-TMC/Gly(90/10)900 polymer phase separates upon addition of water. The amount of water added to effect phase separation represented less than 10% of the total mixture for most PEG400-TMC/Gly(90/10)900/PEG 300 mixtures and decreased as the PEG400-TMC/Gly(90/10)900 content increased. Mixtures containing less than 1% did not undergo phase separation until greater than 10% water was present. The phase separation is expected to form a PEG 300-rich phase and a PEG400-TMC/Gly(90/10)900-rich phase, the former containing the highest proportion of water. Paclitaxel solubility in each phase was measured. Solubility in the -TMC/Gly(90/10)900 water phase was estimated by determination of the PEG400-TMC/Gly(90/10)900/water partition coefficient for paclitaxel, which is 2000, giving an estimated solubility of 2 mg/ml (based on an aqueous solubility of paclitaxel of 1 μg/ml). Solubility in the PEG 300-rich phase was estimated from co-solvent studies of water/PEG 300 mixtures. The solubility of paclitaxel in PEG400-TMC/Gly(90/10)900 alone (not in contact with water) was estimated by visual saturation of the polymer with the drug as 250 mg/ml.

### Example 6

### Preparation of a Paclitaxel Triblock Gel Injection Formulation

A procedure is described that may be used to prepare triblock copolymer compositions loaded a non-ionic, hydrophobic drug (e.g., paclitaxel). The formulations may be administered to a patient via injection. A polymer blend was prepared by dispensing 3 g of PEG400-(90/10 mol % trimethylene carbonate/glycolide)900 and 117 g of PEG300 into a beaker. The components were stirred for at least 2 hours. In a separate beaker, 15 mg of paclitaxel was dispensed and 100 ml of the blended components were added to the paclitaxel and stirred for at least 2 hours. The paclitaxel solution was then withdrawn into a large syringe. A 0.2 μm cellulose acetate syringe filter and a sterile Luer-Lok union was attached to the syringe and then 3 ml syringes were filled with 1.2 ml of paclitaxel loaded triblock copolymer gel solution.

### Example 7

### Cream Formulations

A cream formulation was prepared by heating to 75-80° C. in two 1) water in a first beaker and 2) copolymer, cetyl alcohol, and the surfactant (glyceryl stearate and PEG-75 stearate) in a second beaker. The hydrophobic (oil) phase was thoroughly mixed and slowly added into the water phase with stirring at about 1200 rpm at 75° C. or higher. The oil and water mixture was mixed continuously for 10 minutes at 75° C. and 1200 rpm. The formulation was slowly cooled to ambient temperature under slow stirring to form a cream. The cream is stable at room temperature for at least 5 months.

Formulation A

| Component | % w/w |
|---|---|
| DI Water | 73.5 |
| PEG-TMC copolymer | 20.5 |
| Cetyl alcohol | 4.0 |
| Glyceryl stearate (and) PEG-75 stearate | 2.0 |

Formulation B

| Component | % w/w |
|---|---|
| DI Water | 73.5 |
| ABA Triblock Copolymer | 20.5 |
| A = 1200 g/mol polytrimethylene carbonate, | |
| B = 200 g/mol PEG | |
| Cetyl Alcohol | 4.0 |
| Glyceryl stearate (and) PEG-75 stearate | 2.0 |

### Example 8

### Viscous Cream Formulation

A viscous cream was prepared from the following components using the procedure described in Example 7.

| Component | % w/w |
|---|---|
| DI Water | 73.5 |
| ABA Triblock copolymer | 20.5 |
| A = 900 g/mol polytrimethylene carbonate | |
| B = 200 g/mol PEG | |
| Cetyl alcohol | 4.0 |
| Glyceryl Stearate (and) PEG-75 stearate | 2.0 |

### Example 9

### Lotion Formulation I

A thin, stable cream (lotion) having large droplet size was prepared from the following components using the procedure described in Example 7.

| Component | % w/w |
|---|---|
| DI Water | 81.16 |
| ABA Triblock copolymer | 14.40 |
| A = 900 g/mol polytrimethylene carbonate, | |
| B = 200 g/mol PEG | |
| Cetyl alcohol | 2.90 |
| Glyceryl stearate (and) PEG-75 stearate | 1.54 |

### Example 10

### Comparative Cream Formulation I

A formulation was prepared using the components given below according to the procedure described in Example 7. The formulation formed a thin emulsion with large droplet size, and the phases separated after two days.

| Component | % w/w |
|---|---|
| DI Water | 62.54 |
| Mineral Oil (white, heavy) | 31.80 |
| ABA Triblock copolymer (VISCOPRENE I, Lot 10, | 3.46 |
| supplied by Poly-Med, Inc.) | |
| A = 933 g/mol 90% mol/mol TMC; 10% mol/mol | |
| glycolide copolymer | |
| B = 400 g/mol PEG | |
| Glyceryl stearate (and) PEG-75 stearate | 2.20 |

### Example 11

### Comparative Cream Formulation II

A formulation was prepared with the following components according to the procedure described in Example 7. The formulation formed a thin, liquid emulsion, and the phases separated after several hours.

| Component | % w/w |
|---|---|
| DI Water | 83.76 |
| White petrolatum | 10.81 |
| ABA Triblock copolymer (VISCOPRENE I, Lot 10, | 3.32 |
| supplied by Poly-Med, Inc.) | |
| A = 933 g/mol 90% mol/mol trimethylene carbonate; | |
| 10% mol/mol glycolide copolymer, | |
| B = 400 g/mol PEG | |
| Glyceryl stearate (and) PEG-75 stearate | 2.11 |

### Example 12

### Comparative Cream Formulation III

A formulation was prepared using the following components according to the procedure described in Example 7. The following components were used in the formulation. The formulation formed a thin, liquid emulsion. The phases separated after several hours.

| Component | % w/w |
|---|---|
| DI Water | 63.00 |
| White Petrolatum | 31.67 |
| ABA Triblock copolymer (VISCOPRENE I, Lot 10, | 3.22 |
| supplied by Poly-Med, Inc.) | |
| A = 933 g/mol 90% mol/mol Trimethylene carbonate & | |
| 10% mol/mol glycolide copolymer, B = 400 g/mol PEG | |
| Glyceryl stearate (and) PEG-75 stearate | 2.11 |

### Example 13

### Preparation of an O/W Dispersion of Block Copolymer

Oil in water (o/w) dispersions having the following amounts of triblock gel and water were prepared: (A) triblock gel (1 g): water (9 g) and (B) triblock gel (2 g): water (8 g). After combining the components, the mixtures were shaken by hand for 30 seconds to produce a milky liquid. The resultant copolymer droplets had an average diameter of 200 nm (measured by the MASTERSIZER 2000 (Malvern Instruments) using a HYDRO2000S sample introduction system). The product was suitable for injection without further modification. The milky, macroscopically homogeneous appearance was maintained for several hours upon storage of the dispersion. The product was readily resuspendable with mild hand shaking to mix the partly settled copolymer phase for at least 10 days or longer.

The method may be used to produce o/w dispersions of other water immiscible liquid copolymers and to incorporate a bioactive agent (e.g., a hydrophobic agent) into the dispersion.

### Example 14

### Particle Size Characterization of Dispersions

10 ml dispersions were prepared with and without paclitaxel. Aliquots of triblock gels (having 10% block copolymer PEG400-90/10 TMC/Gly 900) prepared in a manner similar to that described in Example 6. Between 0.5 and 4 ml of the triblock gel was combined with varying volumes of water and mixed by hand-shaking the test tube for thirty seconds.

The dispersion particle sizes of the block copolymer phase were evaluated using a MASTERSIZER 2000 (Malvern Instruments) using a HYDRO2000S sample introduction system. Paclitaxel loaded dispersions were also evaluated by optical microscopy at 400× magnification. Small droplets were visible but no evidence of drug crystals was found.

Particle size data for the dispersion of block copolymer in an aqueous medium with paclitaxel are summarized in the Table 5 (values are the mean of three measurements per sample).

TABLE 5
PARTICLE SIZE DATA FOR THE DISPERSION OF BLOCK
COPOLYMER IN AN AQUEOUS MEDIUM WITH PACLITAXEL

| | Surface | Volume |
|---|---|---|
| | weighted | weighted |
| | mean diameter | mean diameter |
| Composition | (nm) | (nm) |
| 1 ml Triblock Gel 1 mg/ml paclitaxel | 192 | 206 |
| combined with 9 ml water | | |
| 2 ml Triblock Gel 1 mg/ml paclitaxel | 283 | 316 |
| combined with 8 ml water | | |
| 1 ml Triblock Gel 2 mg/ml paclitaxel | 195 | 212 |
| combined with 9 ml water | | |
| 2 ml Triblock Gel 2 mg/ml paclitaxel | 271 | 303 |
| combined with 8 ml water | | |
| 0.5 ml Triblock Gel - no paclitaxel | 187 | 200 |
| combined with 9.5 ml water | | |
| 1 ml Triblock Gel - no paclitaxel | 193 | 208 |
| combined with 9 ml water | | |
| 2 ml Triblock Gel - no paclitaxel | 299 | 329 |
| combined with 8 ml water | | |
| 4 ml Triblock Gel - no paclitaxel | 624 | 1386 |
| combined with 6 ml water | | |

The data show that sub-micron dispersions may be conveniently formed, and that the drug is encapsulated to the extent that no drug crystals form. The data also show that the dispersion size is independent of drug loading, but increases in size and size distribution (the ratio of volume:surface weighted mean diameters) as the triblock gel proportion increased in the composition.

### Example 15

### Preparation of an O/W Dispersion of a Liquid Block Copolymer in an Aqueous Hydrogel Medium Containing Cross-Linked Water Soluble Polymers

Aliquots of 0.1 or 0.2 ml of triblock gel (containing 10% block copolymer PEG400-90/10 TMC/Gly 900) prepared in a manner similar to that described in Example 6 were combined with the acidic solution component of COSEAL (supplied by Baxter Corporation, 2 ml kit). The triblock gel was loaded into a 1 ml syringe and connected to the syringe containing the HCl solution from the COSEAL kit using the supplied connector. The triblock gel was injected into the acid component and the two components were mixed by transferring the liquids back and forth between the syringe barrels at least ten times until a white, macroscopically homogeneous dispersion was formed. The dispersion was left in the acid syringe from the kit. The PEG components from the COSEAL kit were dissolved with the dispersion in the acid syringe by attaching the PEG component syringe with the Luer-Lok connector and mixing the components between the syringes with 25 passages back and forth to ensure the PEGs were dissolved. The resulting solution of PEG components in the dispersion was inserted into the COSEAL spray apparatus, with the second syringe containing the activating basic buffer. The two liquids were ejected through the supplied spray tip without using any compressed gas to facilitate spraying. The two liquid components mixed in the spraying process and formed a hydrogel containing the block copolymer in a dispersed state. The final composition had a white, macroscopically homogeneous appearance.

### Example 16

### Cream Loaded with Hydrophobic Bioactive Agent

A low molecular weight triblock copolymer (e.g., VISCOPRENE I) with lactide, trimethylene carbonate and ethylene glycol units is mixed with polyethylene glycol 300 in a 1:1 ratio. This blend is mixed with 4% aqueous carboxymethyl cellulose solution to form a cream (1:1 ratio). The cream can adhere to the skin and a wet gelatin surface. A highly hydrophobic bioactive agent (e.g., PTX) is dissolved in the triblock polymer blend in a 30 mg/mL concentration. The hydrophobic agent does not precipitate after mixing with the carboxymethyl cellulose solution. Creams made with the following hydrophobic bioactive agents also may be prepared using this procedure: hydrophobic vitamins, such as Vitamins A, D, E, and K; geldanamycin and derivatives (e.g., 17-AAG and 17-DMAG); hydrophobic esters of antibiotics such as erythromycin, ethyl succinate, and erythromycin stearate; anticancer agents such as etoposide, steroid hormones, and antifungal agents such as nystatin and amphotericin.

### Example 17

### Cream Loaded with Hydrophilic Bioactive Agent

A low molecular weight triblock copolymer (e.g., VISCOPRENE I) with lactide, trimethylene carbonate and ethylene glycol units is mixed with polyethylene glycol 300 in a 1:1 ratio. This blend is mixed with 4% aqueous carboxymethyl cellulose solution to form a cream (1:1 ratio). The cream can adhere to the skin and a wet gelatin surface. A hydrophilic bioactive agent (e.g., silk or talc) is dissolved in the triblock polymer blend at a concentration, such that the agent does not precipitate after mixing with the carboxymethyl cellulose solution.

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

From the foregoing it will be appreciated that, although specific embodiments of the invention have been described herein for purposes of illustration, various modifications may be made without deviating from the spirit and scope of the invention. Accordingly, the invention is not limited except as by the appended claims.

1. A method of treating fibrosis at a joint comprising administering to a patient in need thereof a composition comprising.
(a) a block copolymer comprising one or more blocks A and blocks B, wherein
(i) block B is more hydrophilic than block A,
(ii) the block copolymer has a molecular weight, Mn, of between about 500 g/mol and about 2000 g/mol;
(b) a non-polymeric additive; and
(c) a fibrosis-inhibiting agent,
wherein the composition is non-thermoreversible and is a liquid or semi-solid between about 20° C. to about 40° C.

2. The method of claim 1 wherein the copolymer is a triblock copolymer having an ABA or a BAB configuration or a diblock copolymer having an AB configuration.

3-7. (canceled)

8. The method of claim 1 wherein the B block comprises a polyether.

9. The method of claim 8 wherein the B block comprises a polymer selected from polyethylene glycol, polypropylene glycol, poly(1-4-butanediol) and copolymers thereof.

10. The method of claim 1 wherein the A block comprises a polyester, polyether, polyamide or a copolymer thereof.

11. The method of claim 10 wherein the A block copolymer is prepared from one or more of the monomers selected from D lactide, D,L-lactide, L-lactide, glycolide, ε-caprolactone, 6 and γ valerolactone, butyrolactone, 6-decanolactone, 1,4-dioxane-2-one, 1,5-dioxepan-2-one, trimethylene carbonate and caprolactam.

12. The method of claim 1 wherein the A block comprises a polyester, a polycarbonate or a polyester/polycarbonate copolymer, and the B block comprises a water soluble polyether.

13-17. (canceled)

18. The method of claim 1 wherein the block copolymer has a viscosity of below about 1,000 cP at 35° C.

19. (canceled)

20. The method of claim 1 wherein the block copolymer is water insoluble.

21-29. (canceled)

30. The method of claim 1 wherein the block copolymer comprises less than 50% w/w of the composition.

31-34. (canceled)

35. The method of claim 1 wherein the molecular weight of the block copolymer is 2000 g/mol or less.

36-38. (canceled)

39. The method of claim 1 wherein the A blocks have molecular weights that range from between about 100 to about 2000 g/mol.

40. (canceled)

41. The method of claim 1 wherein the B blocks have molecular weights that range from between about 100 to about 2000 g/mol.

42. (canceled)

43. The method of claim 1 wherein the non-polymeric additive is an oligomer.

44. (canceled)

45. The method of claim 43 wherein the oligomer is PEG, PPG, PEG derivative, PPG derivative or copolymers thereof, wherein each of PEG, PPG, PEG derivative, PPG derivative or copolymers thereof has a molecular weight of less than 500 g/mol.

46-47. (canceled)

48. The method of claim 1 wherein the non-polymeric additive is a surfactant.

49-51. (canceled)

52. The method of claim 1 wherein the non-polymeric additive is water.

53. The method of claim 1, wherein the copolymer, the optional non-polymeric additive and the fibrosis-inhibiting agent form a first phase, and the composition further comprises a second phase.

54-56. (canceled)

57. The method of claim 53 wherein the second phase is in the form of a solid, semi-solid, a hydrogel or gel.

58-59. (canceled)

60. The method of claim 53 wherein the second phase comprises water.

61-63. (canceled)

64. The method of claim 1 wherein the composition is in the form of a cream, lotion, or gel.

65-68. (canceled)

69. The method of claim 1 wherein the viscosity of the composition is less than 3000 cP at 25° C.

70-77. (canceled)

78. The method of claim 1 wherein the copolymer is an ABA triblock copolymer, wherein the B block comprises a polyalkylene oxide having a molecular weight of between about 200 g/mol to about 600 g/mol, and the A blocks comprise a polymer having about a 90:10 mole ratio of trimethylene carbonate (TMC) and glycolide (Gly) residues and have a total molecular weight of about 900 g/mol.

79. The method of claim 78 wherein the ABA triblock copolymer, the non-polymeric additive and the fibrosis-inhibiting agent form a first phase, and wherein, the composition further comprises a second phase.

80. The method of claim 79, wherein the second phase comprises water soluble polysaccharide, a polyethylene glycol, alcohol or water.

81. The method of claim 79, wherein the non-polymeric additive is PEG 300.

82-86. (canceled)

87. The method of claim 1 wherein the fibrosis-inhibiting agent is a taxane.

88. The method of claim 1 wherein fibrosis-inhibiting agent is one or more steroids.

89. The method of claim 1 wherein the composition is delivered to the joint by intra-articular injection.

90-118. (canceled)

119. The method of claim 1 wherein treating fibrosis at a joint includes treating inflammatory arthritis.

120. The method of claim 87 wherein the fibrosis-inhibiting agent is paclitaxel.
